(19) United States
(12) Patent Application Publication
(10) Pub. No.: US 2025/0368783 A1
(43) Pub. Date: Dec. 4, 2025

(54) DITHIOL-BASED POLYMERS AND CYCLIC COMPOUNDS AND METHODS FOR MAKING AND USING THE SAME

(71) Applicant: BOARD OF REGENTS OF THE NEVADA SYSTEM OF HIGHER EDUCATION, ON BEHALF OF THE UNIVERSITY, Reno, NV (US)

(72) Inventors: Ying Yang, Reno, NV (US); Lasith S. Kariyawasam, Reno, NV (US)

(73) Assignee: BOARD OF REGENTS OF THE NEVADA SYSTEM OF HIGHER EDUCATION, ON BEHALF OF THE UNIVERSITY, Reno, NV (US)

(21) Appl. No.: 18/874,358

(22) PCT Filed: Jun. 12, 2023
(86) PCT No.: PCT/US2023/025058

§ 371 Date: Dec. 12, 2024

Related U.S. Application Data
(60) Provisional application No. 63/351,724, filed on Jun. 13, 2022.

(51) Int. Cl. C08G 75/10; C08G 75/0286

(52) U.S. Cl. CPC C08G 75/10; C08G 75/0286

(57) ABSTRACT

Disclosed herein are dithiol-based polymers and cyclic compounds that can be formed therefrom. The cyclic compounds also can be used to regenerate recycled polymers. Methods for making native and recycled polymers are described herein, along with methods for using the compounds of the present disclosure to provide crosslinked “vitrimer”-like products.

### CROSS REFERENCE TO RELATED APPLICATION

This application claims the benefit of and priority to U.S. Provisional Patent Application No. 63/351,724 filed on Jun. 13, 2022, the entirety of which is incorporated herein by reference.

### FIELD

The present disclosure is directed to dithiol-based polymers and cyclic compounds and methods of making and using the same.

### BACKGROUND

Global plastic waste production continues to accelerate at an alarming rate introducing grave environmental effects that urgently warrants producing degradable/recyclable plastics. In this regard, dynamic covalent polymers are an active area of research to develop polymers capable of chemical recycling to monomers (CRM). A need exists in the art, however, for new polymer materials that can be converted to monomeric components using efficient methods and wherein the monomeric components are capable of being converted back to recycled forms of the polymers.

### SUMMARY

Disclosed herein are embodiments of a linear polymer having a structure according to Formula I, as described herein, wherein ring A is an aromatic ring system; each R independently is selected from aliphatic, aromatic, heteroaliphatic, or any combination thereof; or each R independently is selected from an R′ group, wherein R′ has a structure according to a Formula A, —R^{c}—S—R^{d}—S—R^{c}—, wherein each R^{c} is selected from the groups recited for R, and R^{d} is a group having a Formula B, —R^{c}—S[CH₂]_{q}O—Ar—C(Me₂)-Ar—O[CH₂]_{q}S—R^{c}—, or a Formula C, —R^{c}—S[CH₂]_{q}Y[CH₂]_{q}S—R^{c}—, wherein for Formula B, each Ar is an aromatic ring system; for both Formulas B and C, each q independently is an integer selected from 1 to 10; and for Formula C, Y is selected from an amide group, a carbamide group, or a sulfonyl group; each X independently is selected from an electron-withdrawing group or an electron-donating group; n is an integer selected to provide an Mₙ ranging from 5,000 g/mol to 120,000 g/mol; and m is an integer ranging from 0 to 5. In independent embodiments, it is provided that (i) if ring A is phenyl and m is 0, then R is not —(CH₂)₆—, —(CH₂CH₂O)₂CH₂CH₂—, -Ph-S-Ph-; (ii) if ring A is phenyl, m is 1, and X is para-methoxy, then R is not —(CH₂)₆—, —(CH₂CH₂O)₂CH₂CH₂—, -Ph-S-Ph-, —CH₂C(O)O(CH₂)₄OC(O)CH₂—, or —(CH₂)₂C(O)O(CH₂)₄OC(O)(CH₂)₂—; (iii) if ring A is phenyl, m is 1, and X is para-OCH₂CH═CH₂, then R is not —(CH₂CH₂O)₂CH₂CH₂—; (iv) if ring A is phenyl, m is 1, and X is para-OH, then R is not —(CH₂CH₂O)₂CH₂CH₂—; (v) if ring A is phenyl, m is 1, and X is para-Cl, then R is not —(CH₂CH₂O)₂CH₂CH₂—; and/or (vi) if ring A is phenyl, m is 1, and X is para-NO₂, then R is not —(CH₂CH₂O)₂CH₂CH₂—.

Also disclosed herein are embodiments of a cyclic dithioacetal compound having a structure according to Formula II as described herein, wherein ring A is an aromatic ring system; each R independently is selected from aliphatic, aromatic, heteroaliphatic, or any combination thereof; or each R independently is selected from an R′ group, wherein R′ has a structure according to a Formula A, —R^{c}—S—R^{d}—S—R^{c}—, wherein each R^{c} is selected from the groups recited for R, and R^{d} is a group having a Formula B, —R^{c}—S[CH₂]_{q}O—Ar—C(Me₂)-Ar—O[CH₂]_{q}S—R^{c}—, or a Formula C, —R¹—S[CH₂]_{q}Y[CH₂]_{q}S—R^{c}—, wherein for Formula B, each Ar is an aromatic ring system; for both Formulas B and C, each q independently is an integer selected from 1 to 10; and for Formula C, Y is selected from an amide group, a carbamide group, or a sulfonyl group; each X independently is selected from an electron-withdrawing group or an electron-donating group; n′ is an integer ranging from 1 to 8; and m is an integer ranging from 0 to 5. In independent embodiments, it is provided that (i) if ring A is phenyl and m is 0, then R is not —(CH₂)₆—, —(CH₂CH₂O)₂CH₂CH₂—, -Ph-S-Ph-; (ii) if ring A is phenyl, m is 1, and X is para-methoxy, then R is not —(CH₂)₆—, —(CH₂CH₂O)₂CH₂CH₂—, -Ph-S-Ph-, —CH₂C(O)O(CH₂)₄OC(O)CH₂—, or —(CH₂)₂C(O)O(CH₂)₄OC(O)(CH₂)₂—; (iii) if ring A is phenyl, m is 1, and X is para-OCH₂CH═CH₂, then R is not —(CH₂CH₂O)₂CH₂CH₂—; (iv) if ring A is phenyl, m is 1, and X is para-OH, then R is not —(CH₂CH₂O)₂CH₂CH₂—; (v) if ring A is phenyl, m is 1, and X is para-Cl, then R is not —(CH₂CH₂O)₂CH₂CH₂—; and/or (vi) if ring A is phenyl, m is 1, and X is para-NO₂, then R is not —(CH₂CH₂O)₂CH₂CH₂—.

Also disclosed herein are embodiments of a method for making a linear polymer as described herein, wherein the method comprises: exposing a dithiol monomer component and an aldehyde-containing monomer component to an acid catalyst to form a reaction mixture; and exposing the reaction mixture to a reaction temperature; wherein the dithiol monomer component has a structure according to Formula III, HS—R—SH, wherein R is as according any or all of the above embodiments; and the aldehyde-containing monomer component has a structure according to Formula IV, as described herein wherein ring A, X, and m are as recited for any or all of the above embodiments.

Also disclosed herein are embodiments of a method for making a cyclic dithioacetal compound, comprising: exposing a polymer according to any embodiments described herein to an acid catalyst to provide a reaction mixture; and exposing the reaction mixture to reaction temperature; wherein the cyclic dithioacetal compound has a structure according to Formula II as described herein, wherein substituents for Formula II are as described for any or all of the above embodiments. In independent embodiments, it is provided that (i) if ring A is phenyl and m is 0, then R is not —(CH₂)₆—, —(CH₂CH₂O)₂CH₂CH₂—, -Ph-S-Ph-; (ii) if ring A is phenyl, m is 1, and X is para-methoxy, then R is not —(CH₂)₆—, —(CH₂CH₂O)₂CH₂CH₂—, -Ph-S-Ph-, —CH₂C(O)O(CH₂)₄OC(O)CH₂—, or —(CH₂)₂C(O)O(CH₂)₄OC(O)(CH₂)₂—; (iii) if ring A is phenyl, m is 1, and X is para-OCH₂CH═CH₂, then R is not —(CH₂CH₂O)₂CH₂CH₂—; (iv) if ring A is phenyl, m is 1, and X is para-OH, then R is not —(CH₂CH₂O)₂CH₂CH₂—; (v) if ring A is phenyl, m is 1, and X is para-Cl, then R is not —(CH₂CH₂O)₂CH₂CH₂—; and/or (vi) if ring A is phenyl, m is 1, and X is para-NO₂, then R is not —(CH₂CH₂O)₂CH₂CH₂—.

Also disclosed herein are embodiments for making a recycled polymer, comprising exposing a cyclic dithioacetal compound according to any or all of the above embodiments to an acid catalyst to provide the recycled polymer.

Also disclosed herein are embodiments of a method, comprising exposing a linear polymer having a structure according to Formula I to a first acid catalyst to promote ring-closing depolymerization to provide a cyclic dithioacetal compound having a structure according to Formula II; and exposing the cyclic dithioacetal compound to reaction conditions sufficient to promote entropy-driven ring-opening polymerization to provide a recycled linear polymer having a structure according to Formula I and its recited substituents are as recited herein and wherein Formula II and its substituents are as recited herein.

The foregoing and other objects, features, and advantages of the present disclosure will become more apparent from the following detailed description, which proceeds with reference to the accompanying figures.

### BRIEF DESCRIPTION OF THE DRAWINGS

FIGS. 1A and 1B are ¹H NMR spectrum (500 MHz, CDCl₃) and ¹³C{¹H} NMR spectrum (126 MHz, CDCl₃), respectively, of native linear polymer P1.

FIGS. 2A and 2B are ¹H NMR spectrum (500 MHz, CDCl₃) and ¹³C{¹H} NMR spectrum (126 MHz, CDCl₃), respectively, of native linear polymer P2.

FIGS. 3A and 3B are ¹H NMR spectrum (500 MHz, CDCl₃) and ¹³C{¹H} NMR spectrum (126 MHz, CDCl₃), respectively, of native linear polymer P3.

FIG. 4 shows results obtained from monitoring the reaction between 3,4,5-trimethoxybenzaldehyde and 1,6-hexanedithiol to form native linear polymer P1 using ¹H NMR spectroscopy, wherein the topmost spectrum corresponds to the purified native linear polymer, and the underlying spectra are for unpurified reaction mixtures quenched with triethylamine at the indicated times.

FIGS. 5A-5C provide summaries of the reaction for making native linear polymer P1, wherein FIG. 5A shows a plot of percent conversion of 3,4,5-trimethoxybenzaldehyde monomer to the polymer as determined by ¹H NMR after quenching aliquots of the reaction mixture with triethylamine at different times; FIG. 5B shows GPC traces for the formation of P1 at different times; and FIG. 5C is a plot of Mₙ as a function of conversion of the monomer, wherein data points for theoretical values predicted by Carothers equation and experimental values obtained by GPC are included.

FIGS. 6A-6C provide summaries of the reaction for making native linear polymer P3, wherein FIG. 6A shows a plot of percent conversion of 3,4,5-trimethoxybenzaldehyde monomer to the polymer as determined by ¹H NMR after quenching aliquots of the reaction mixture with triethylamine at different times; FIG. 6B shows GPC traces for the formation of P3 at different times; and FIG. 6C is a plot of Mₙ as a function of conversion of the monomer, wherein data points for theoretical values predicted by Carothers equation and experimental values obtained by GPC are included.

FIGS. 7A-7C show DSC curves for P1 (FIG. 7A), P2 (FIG. 7B), and P3 (FIG. 7C) measured from the second heating cycle.

FIG. 8 is a combined graph showing TGA curves of purified polymers P1, P2, and P3.

FIG. 9 shows combined GPC traces for unpurified linear polymer P1 over time.

FIG. 10 shows combined GPC traces for unpurified linear polymer P2 over time.

FIG. 11 shows combined GPC traces for unpurified linear polymer P3 over time.

FIG. 12 provides an illustrative scheme showing the reversible conversion of representative linear polymers to cyclic dithioacetal compounds using methods disclosed herein.

FIG. 13 includes photographic images of the different products obtained from the reversible conversion shown in FIG. 12.

FIGS. 14A-14F show characterization results for ring-chain recycling of dithioacetal-based linear polymers and cyclic dithioacetal compounds via reversible ring-closing depolymerization (RCD) and ring-opening polymerization (ROP), wherein FIGS. 14A and 14B are full and partial ¹H NMR spectra of native polymer P1 (wherein the lower spectrum is for the polymer, the middle spectrum is for the crude mixture of cyclic dithioacetal compounds, and the upper spectrum is for the recycled polymer); FIGS. 14C and 14D are full and partial ¹H NMR spectra native polymer P2 (wherein the lower spectrum is for the polymer, the middle spectrum is for the crude mixture of cyclic dithioacetal compounds, and the upper spectrum is for the recycled polymer); and FIGS. 14E and 14F are full and partial ¹H NMR spectra P3 (wherein the lower spectrum is for the polymer, the middle spectrum is for the crude mixture of cyclic dithioacetal compounds, and the upper spectrum is for the recycled polymer).

FIGS. 15A-15C show GPC traces showing the normalized UV (254 nm) response as a function of retention time for polymers P1 (FIG. 15A), P2 (FIG. 15B), and P3 (FIG. 15C), wherein the labels correspond to the native polymer, the cyclic dithioacetal compound mixture, and the recycled polymer.

FIG. 16 is a plot showing a summary of RCD as a function of time for native linear polymers P1, P2, and P3.

FIGS. 17A-17C show ¹H NMR spectra (500 MHz, CDCl₃) for the products obtained from exposing P1 (FIG. 17A), P2 (FIG. 17B), and P3 (FIG. 17C) to RCD.

FIG. 18 is a GPC trace for the RCD product mixture obtained from exposing native linear polymer, P2 to RCE, at different reaction times.

FIGS. 19A and 19B show full (FIG. 19A) and zoomed (FIG. 19B) GPC traces obtained from exposing native linear polymer P1 to RCD.

FIGS. 20A and 20B show full (FIG. 20A) and zoomed (FIG. 20B) GPC traces obtained from exposing native linear polymer P2 to RCD.

FIGS. 21A and 21B show full (FIG. 21A) and zoomed (FIG. 21B) GPC traces obtained from exposing native linear polymer P3 to RCD.

FIG. 22 shows the MALDI-TOF MS spectrum obtained from exposing native linear polymer P1 to RCD.

FIG. 23 shows the MALDI-TOF MS spectrum obtained from exposing native linear polymer P2 to RCD.

FIGS. 24A-24D show characterization results for the cyclic dimer (m=0) obtained from RCD of native linear polymer P3, wherein FIG. 24A is the ¹H NMR spectrum (500 MHz, CDCl₃); FIG. 24B is the ¹³C{¹H} NMR spectrum (126 MHz, CDCl₃); FIG. 24C is the GPC trace; and FIG. 24D is the MALDI-TOF spectrum.

FIGS. 25A-25C show characterization results for the cyclic tetramer (m=1) obtained from the RCD of native linear polymer P3, wherein FIG. 25A is the ¹H NMR spectrum (500 MHz, CDCl₃); FIG. 25B is the ¹³C{¹H} NMR spectrum (126 MHz, CDCl₃); and FIG. 25C is the GPC trace.

FIGS. 26A and 26B show the ¹H NMR spectrum (500 MHz, CDCl₃) and ¹³C{¹H} NMR spectrum (126 MHz, CDCl₃), respectively, for another cyclic dithioacetal compound obtained from RCD of native linear polymer P3.

FIG. 27 is a plot of percent conversion of RCD mixtures to the corresponding recycled polymers obtained from ROP as a function of time, as determined by ¹H NMR.

FIGS. 28A-28C show the ¹H NMR spectra (500 MHz, CDCl₃) of the P1-RCD mixture after ROP (FIG. 28A), the P2-RCD mixture after ROP (FIG. 28B), and the P3-RCD mixture after ROP (FIG. 28C).

FIGS. 29A and 29B show the GPC traces for the P1-RCD mixture after ROP (before purification) and after different reaction times (FIG. 29A); and a comparison of the mixtures before and after purification (FIG. 29B).

FIGS. 30A and 30B show the GPC traces for the P2-RCD mixture after ROP (before purification) and after different reaction times (FIG. 30A); and a comparison of the mixtures before and after purification (FIG. 30B).

FIGS. 31A and 31B show the GPC traces for the P2-RCD mixture after ROP (before purification) and after different reaction times (FIG. 31A); and a comparison of the mixtures before and after purification (FIG. 31B).

FIG. 32 shows the enlarged ¹H NMR (CDCl₃) of the tertiary dithioacetal protons on the benzylic carbon of the RCD mixture of native linear polymer P1 (bottom spectrum) and native linear polymer P1 formation over time, wherein in some aspects of the disclosure, the RCD of the polymer yields predominantly a mixture of cyclic dithioacetal compounds, while the polymer formation presumably occurs via linear oligomers in a step-growth pathway and in the beginning of P1 formation, particularly at t=1 min, linear oligomers are predominantly present and toward the end, linear P1 becomes dominant over time.

FIG. 33 shows GPC curves for the RCD mixture of native linear polymers P1 (left image) and P3 (right image) (indicated with dotted lines in both images) and the formation of native linear polymers P1 and P3 at t=1 min (indicated with solid lines in both images).

FIGS. 34A and 34B show ¹H NMR spectrum (400 MHz, CDCl₃) of phenyl-thiocarbamate tethered poly(dithioacetal) (FIG. 34A) and bis-phenyl-thiocarbamate tethered poly(dithioacetal) (FIG. 34B).

FIG. 35 shows results from using ¹H NMR (CDCl₃) to monitor the reaction of ROP of the cyclic dithioacetal dimer of P3 at room temperature after quenching aliquots of the reaction mixture with triethylamine at different times, where the [initial monomer]=0.50 mol L⁻¹ and [PTSA]=0.25 mol L⁻¹.

FIG. 36 shows a plot of the percent conversion of the cyclic dithioacetal dimer of P3 to the recycled polymer at room temperature as determined by ¹H NMR.

FIG. 37 shows GPC (THF) monitoring of the triethylamine quenched reaction run at room temperature at different times indicated for the ROP of the cyclic dithioacetal dimer of P3, where the [initial monomer]=0.50 mol L⁻¹ and [PTSA]=0.25 mol L⁻¹.

FIG. 38 is a plot of Mₙ and Ð as a function of conversion of an aldehyde-based monomer component to native linear polymer P3 at rt, where the [initial monomer]=0.50 mol L⁻¹ and [PTSA]=0.25 mol L⁻¹.

FIG. 39 is a plot of percent conversion of an aldehyde-based monomer component to native linear polymer P3 at room temperature as determined by ¹H NMR, where [initial monomer]=0.08 mol L⁻¹ and [PTSA]=0.25 mol L⁻¹.

FIG. 40 shows the results from using GPC (THF) monitoring of the triethylamine quenched reaction run at room temperature at different times wherein an aldehyde-based monomer component was converted to native linear polymer P3, where the [initial monomer]=0.08 mol L⁻¹ and [PTSA]=0.25 mol L⁻¹.

FIG. 41 is a plot of Mₙ and Ð as a function of conversion of an aldehyde-based monomer component to native linear polymer P3 at rt, where the [initial monomer]=0.08 mol L⁻¹ and [PTSA]=0.25 mol L⁻¹.

FIG. 42 is a plot of percent conversion of an aldehyde-based monomer component to polymer P3 as a function of time at room temperature for two different initial monomer concentrations [M]₀ (0.50 and 0.08 mol L⁻¹).

FIGS. 43A and 43B are GPC traces showing the normalized UV (254 nm) response as a function of retention time for the above polymerization at rt, where FIG. 43A shows results for [M]₀=0.50 mol/L and FIG. 43B shows results for [M]₀=0.08 mol/L.

FIG. 44 shows a schematic illustration of the reprocessing and recycling mechanisms for a crosslinked polymer.

FIG. 45 provides photographic images of results obtained from heating and pressing a crosslinked polymer before and after heating/pressing.

FIGS. 46A-46C show stress-strain curves of the original crosslinked P3 films and after 2×, 5×, and 10× recycling cycles (FIG. 46A), normalized shear stress relaxation analysis of the original crosslinked P3 network under various temperatures (FIG. 46B), and Arrhenius plot constructed from the relaxation time (t*) at different temperatures calculated from the stress-relaxation data (FIG. 46C).

FIGS. 47A-47D show results from performing RCD on native linear polymer P3, wherein FIG. 47A shows the ¹H NMR spectra (CDCl₃) of the RCD mixture of native linear polymer P3 (top spectrum) (for a comparison), RCD mixture of crosslinked P3 (middle spectrum), and the soluble fraction of crosslinked P3 (bottom spectrum); FIG. 47B shows the zoomed-in ¹H NMR spectra (CDCl₃) of the tertiary dithioacetal protons on the benzylic carbon of the RCD mixture of native linear polymer P3 (top spectrum) (for a comparison), RCD mixture of crosslinked P3 (middle spectrum), and the soluble fraction of crosslinked P3 (bottom spectrum); FIG. 47C shows the ¹H NMR spectra (CDCl₃) of soluble fraction of the regenerated crosslinked P3 after subjecting the RCD mixture of crosslinked P3 to undergo ROP (top spectrum) and RCD mixture of crosslinked P3 (bottom spectrum); and FIG. 47D shows the zoomed-in ¹H NMR spectra (CDCl₃) of the tertiary dithioacetal protons on the benzylic carbon of the soluble fraction of the regenerated crosslinked P3 after subjecting the RCD mixture of crosslinked P3 to undergo ROP (top spectrum) and RCD mixture of crosslinked P3 (bottom spectrum).

FIG. 48 shows GPC traces for the soluble fraction of crosslinked P3, after depolymerization of crosslinked P3, and the soluble fraction after repolymerization to reform crosslinked P3.

FIG. 49 shows DSC curves for recycled crosslinked P3 measured from the second heating cycle after 2× and 10× recycling.

FIG. 50 shows the ¹H NMR (CDCl₃) of the RCD mixture of crosslinked P3 (containing zinc(II) triflate).

FIG. 51 shows stress-strain curves of the original crosslinked P3 films (containing zinc(II) triflate), after chemical reprocessing including RCD and ROP, followed by three times of heat reprocessing.

FIGS. 52A and 52B show ¹H NMR spectra (FIG. 52A) and GPC traces (FIG. 52B) for ring-chain recycling (including polymerization to the native linear polymer and the reversible RCD and ROP reactions) of a representative homopolymer.

FIGS. 53A-53C show results obtained from ¹H NMR monitoring of copolymer formation using racemic CSA (FIGS. 53A and 53B) and different acid catalysts (FIG. 53C).

FIGS. 54A and 54B show ¹H NMR spectra (FIG. 54A) and GPC traces (FIG. 54B) for ring-chain recycling (including polymerization to the native linear polymer and the reversible RCD and ROP reactions) of a copolymer compound using a particular ratio of the monomer components of the copolymer.

FIGS. 55A and 55B show ¹H NMR spectra (FIG. 55A) and GPC traces (FIG. 55B) for ring-chain recycling (including polymerization to the native linear polymer and the reversible RCD and ROP reactions) of a copolymer compound using a particular ratio of the monomer components of the copolymer.

FIGS. 56A and 56B show ¹H NMR spectra (FIG. 56A) and GPC traces (FIG. 56B) for melt polymerization of the cyclic dithioacetal products obtained from RCD of P1.

### DETAILED DESCRIPTION

### Overview of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements unless the context clearly indicates otherwise.

Although the steps of some of the disclosed methods are described in a particular, sequential order for convenient presentation, it should be understood that this manner of description encompasses rearrangement, unless a particular ordering is required by specific language set forth below. For example, steps described sequentially may in some cases be rearranged or performed concurrently. Additionally, the description sometimes uses terms like “produce” or “provide” to describe the disclosed methods. These terms are high-level abstractions of the actual steps that are performed. The actual steps that correspond to these terms will vary depending on the particular implementation and are readily discernible by one of ordinary skill in the art.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

Certain functional group terms include a symbol “-” which is used to show how the defined functional group attaches to, or within, the donor compound to which it is bound. Also, a dashed bond (i.e., “---”) as used in certain formulas described herein indicates an optional bond (that is, a bond that may or may not be present). A person of ordinary skill in the art would recognize that the definitions provided below and the donor compounds and formulas included herein are not intended to include impermissible substitution patterns (e.g., methyl substituted with 5 different groups, and the like). Such impermissible substitution patterns are easily recognized by a person of ordinary skill in the art. In formulas and donor compounds disclosed herein, a hydrogen atom is present and completes any formal valency requirements (but may not necessarily be illustrated) wherever a functional group or other atom is not illustrated. For example, a phenyl ring that is drawn as

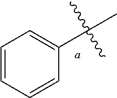 [SMILES: *c1ccccc1]

comprises a hydrogen atom attached to each carbon atom of the phenyl ring other than the “a” carbon, even though such hydrogen atoms are not illustrated. Any functional group disclosed herein and/or defined above can be substituted or unsubstituted, unless otherwise indicated herein.

In any embodiments, any or all hydrogens present in compound embodiments disclosed herein, or in a particular group or moiety within the compound, may be replaced by deuterium or tritium. As an example, recitation of “alkyl” includes deuterated and/or tritiated alkyl, where from one to the maximum number of hydrogens present may be replaced by deuterium and/or tritium. For example, methyl refers to both CH₃, or CH₃ wherein from 1 to 3 hydrogens are replaced by deuterium, such as in CDₓH₃₋ₓ.

To facilitate review of the various embodiments of the disclosure, the following explanations of specific terms are provided.

Aldehyde: —C(O)H.

Aliphatic: A hydrocarbon group having at least one carbon atom to 50 carbon atoms (C₁-C₅₀), such as one to 25 carbon atoms (C₁-C₂₅), or one to ten carbon atoms (C₁-C₁₀), and which includes alkanes (or alkyl), alkenes (or alkenyl), alkynes (or alkynyl), including cyclic versions thereof, and further including straight- and branched-chain arrangements, and all stereo and position isomers as well.

Alkenyl: An unsaturated monovalent hydrocarbon having at least two carbon atoms to 50 carbon atoms (C₂-C₅₀), such as two to 25 carbon atoms (C₂-C₂₅), or two to ten carbon atoms (C₂-C₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., E or Z).

Alkoxy: An exemplary heteroaliphatic group having a formula —O-aliphatic, with exemplary embodiments including, but not limited to, methoxy, ethoxy, n-propoxy, isopropoxy, n-butoxy, t-butoxy, sec-butoxy, n-pentoxy.

Alkyl: A saturated monovalent hydrocarbon having at least one carbon atom to 50 carbon atoms (C₁-C₅₀), such as one to 25 carbon atoms (C₁-C₂₅), or one to ten carbon atoms (C₁-C₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl).

Alkynyl: An unsaturated monovalent hydrocarbon having at least two carbon atoms to 50 carbon atoms (C₂-C₅₀), such as two to 25 carbon atoms (C₂-C₂₅), or two to ten carbon atoms (C₂-C₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl).

Amide: An exemplary heteroaliphatic group having a formula —C(O)NR^{a}R^{b} or —NHCOR^{a} wherein each of R^{a} and R^{b} independently is selected from hydrogen, aliphatic, heteroaliphatic, haloaliphatic, aromatic, or any combination thereof.

Amine: —NR^{a}R^{b}, wherein each of R^{a} and R^{b} independently is selected from hydrogen, aliphatic, heteroaliphatic, haloaliphatic, aromatic, or any combination thereof.

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized Tr-electron system. Typically, the number of out of plane Tr-electrons corresponds to the Hückel rule (4n+2). The point of attachment to the parent structure typically is through an aromatic portion of the condensed ring system. For example,

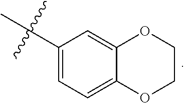 [SMILES: *c1ccc2c(c1)OCCO2]

However, in certain examples, context or express disclosure may indicate that the point of attachment is through a non-aromatic portion of the condensed ring system. For example,

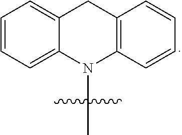 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g., S, O, N, P, or Si), such as in a heteroaryl group or moiety.

Aryl: An aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, hetero-aliphatic, aromatic, other functional groups, or any combination thereof.

Carboxyl: —C(O)OH or an anion thereof.

Disulfide: An exemplary heteroaliphatic group having a formula —SSR^{a}, wherein R^{a} is selected from hydrogen, aliphatic, heteroaliphatic, haloaliphatic, aromatic, or any combination thereof.

Electron-Withdrawing Group: A functional group capable of accepting electron density from an aromatic ring system to which it is directly attached, such as by inductive electron withdrawal.

Electron-Donating Group: A functional group capable of donating at least a portion of its electron density into an aromatic ring system to which it is directly attached, such as by resonance.

Ester: An exemplary heteroaliphatic group having a formula —C(O)OR^{a} or —OC(O)R^{a}, wherein R^{a} is selected from aliphatic, heteroaliphatic, haloaliphatic, aromatic, or any combination thereof.

Haloaliphatic: An aliphatic group wherein one or more hydrogen atoms, such as one to 10 hydrogen atoms, independently is replaced with a halogen atom, such as fluoro, bromo, chloro, or iodo.

Haloalkyl: An alkyl group wherein one or more hydrogen atoms, such as one to 10 hydrogen atoms, independently is replaced with a halogen atom, such as fluoro, bromo, chloro, or iodo. In an independent embodiment, haloalkyl can be a CX₃ group, wherein each X independently can be selected from fluoro, bromo, chloro, or iodo.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group.

Heteroaryl: An aromatic group comprising at least one heteroatom to six heteroatoms, such as one to four heteroatoms, which can be selected from, but not limited to oxygen, nitrogen, sulfur, silicon, boron, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group. Heteroaryl groups may be substituted with one or more groups other than hydrogen, such as aliphatic, heteroaliphatic, aromatic, other functional groups, or any combination thereof.

Heteroatom: An atom other than carbon, such as oxygen, nitrogen, sulfur, silicon, boron, selenium, or phosphorous. In particular disclosed embodiments, such as when valency constraints do not permit, a heteroatom does not include a halogen atom.

Ketone: An exemplary heteroaliphatic group having a formula —C(O)R^{a}, wherein R^{a} is selected from aliphatic, heteroaliphatic, haloaliphatic, aromatic, or any combination thereof.

Native Linear Polymer: A linear polymer having a structure according to formulas described herein that is made by reacting a dithiol monomer component with an aldehyde-containing monomer component. Native linear polymers are different from recycled linear polymers described herein in the sense that the native linear polymer is the direct product of reacting the dithiol monomer component with the aldehyde-containing monomer component, whereas recycled linear polymers are obtained from ED-ROP of a cyclic dithioacetal compound(s) according to the present disclosure. Native linear polymers and recycled linear polymers may have the same chemical structure; however, they are referenced herein as “native” and “recycled” so as to indicate the distinction between the polymers as described in this definition.

Polymer: A molecule having a structure comprising repeating units of at least two monomeric units, wherein the monomeric units are obtained from the reaction of at least one dithiol monomer component and at least one aldehyde-containing monomer component as described herein. The term “polymer” also encompasses oligomers and/or macromolecules unless expressly stated otherwise. In some aspects of the disclosure, a polymer comprises a number of monomeric units sufficient to provide a molecular weight (Mₙ) ranging from 5,000 g/mol to 120,000 g/mol or higher, such as 10,000 g/mol to 120,000 g/mol or higher.

Recycled Linear Polymer: A linear polymer having a structure according to formulas described herein that is made by ED-ROP of a cyclic dithioacetal compound according to the present disclosure. Recycled linear polymers are different from native linear polymers described herein in the sense that the recycled linear polymer is the direct product obtained from ED-ROP of a cyclic dithioacetal compound according to the present disclosure, whereas native linear polymers are made by reacting a dithiol monomer component with an aldehyde-containing monomer component. Recycled linear polymers and native linear polymers may have the same chemical structure; however, they are referenced herein as “recycled” and “native” so as to indicate the distinction between the polymers as described in this definition.

### Introduction

The annual solid waste production around the globe exceeds two billion tons and it is likely to continue rapidly over the next few decades irrespective of the alarming environmental impacts. Thus, the development of degradable and recyclable materials with the overarching goal of reducing plastic pollution is an area of active research. Since the recycled monomers can be used to resynthesize the polymers while virtually retaining the quality of the original polymers, the concept of chemical recycling to monomers (CRM) has gained significant interest in the recent past as a viable strategy to tackle the plastic waste issue. Recently, cyclic monomers have been employed to realize the CRM, where the ring-opening polymerization (ROP) of the cyclic monomers forms the polymers, while the ring-closing depolymerization (RCD) of the resulting polymers affords the cyclic monomers, leading to a reversible process of ring-chain recycling. In this regard, cyclic acetals, cyclic dithioacetals, cyclic carbonates, cycloalkenes, lactones, and thiolactones have been recently used as cyclic monomers.

One dynamic covalent bond that has received only limited attention in the field of polymer chemistry but has been widely used in synthetic organic chemistry is dithioacetal. Although different chemistries can be used to synthesize polydithioacetals, a more straightforward route to synthesize them with high yields would be to react a benzaldehyde derivative with a dithiol in the presence of an acid catalyst. The formation of polydithioacetal begins with the nucleophilic addition of a dithiol into an acid-activated aldehyde species to generate a thiocarbenium intermediate, which then undergoes complex chain-cycle equilibria. Intramolecular cyclization of the intermediate can yield the cyclic dithioacetal dimer. Additionally, it can react with another thiocarbenium ion to afford a longer thiocarbenium species, which in turn can cyclize to form a cyclic tetramer or react with yet another thiocarbenium to yield even longer thiocarbenium species. The acid-catalyzed reversible ring-chain equilibria eventually lead to the generation of the polydithioacetal through oligomerization. Once the acid catalyst is removed, the dithioacetals are quite stable. In short, the C—S bonds in dithioacetals can be activated with a catalytic amount of an acid. Given their straightforward synthesis, tunability, and dynamics, dithioacetal as a reversible bond in developing recyclable polymers was investigated.

The compounds disclosed herein provide several advantages over the art, including (but not limited to): 1) efficient synthesis—polymers can be formed via a one-step reaction between aldehyde and dithiol monomers according to the present disclosure, which then undergo closed-loop recycling via RCD and ROP; 2) structural flexibility—chemical handles for versatile backbone and/or side-chain engineering can be introduced via starting materials described herein, which open tremendous opportunities for functionalization; and 3) stability—compared to acetals known in the art, dithioacetals have much higher hydrolytic stability. Additionally, using ED-ROP in recyclable polymer compounds is described herein, which provides advantages over current methods in the art, such as (i) the ability to use macrocyclic mixtures as monomers for ED-ROPs without having to isolate individual rings; and (ii) carrying out such reactions at ambient temperatures within a short time to yield high molecular weight polymers. This is therefore beneficial compared to ROP methods that are mostly not feasible at or above rt, particularly without exhibiting depolymerization.

### Native and Recycled Linear Polymers and Cyclic Monomers

Native and recycled polymer compounds and methods of making and using the same are described herein. In some aspects of the disclosure, the native and/or recycled polymer compounds are linear polymers that comprise a dithioacetal-based polymer backbone. Native and/or recycled polymer compounds of the present disclosure exhibit superior stability against hydrolysis relative to acetal-based polymers, even at high temperatures (e.g., temperatures of up to 300° C., such as 250° C. to 300° C., or 270° C. to 300° C., or 285° C. to 300° C.). Native linear polymer compounds are made using a dithiol monomer component and an aldehyde-containing monomer component. The dithiol monomer component and the aldehyde-containing monomer component are reacted in the presence of a catalyst (with or without an initiator compound) to form a covalent bond between the dithiol monomer component and the aldehyde-containing monomer compound and which provides the linear polymer backbone of the native linear polymer compound. In certain aspects of the disclosure, the native and/or recycled linear polymer compound can be subjected to further chemical reactions that facilitate one or more of the following: (i) ring-closing depolymerization (or “RCD”) of a native linear polymer so as to form cyclic dithioacetal compounds; (ii) ring-opening polymerization, such as entropy-driven ring-opening polymerization (or “ED-ROP”) wherein the cyclic dithioacetal compounds undergo ring-opening and repolymerization to provide recycled linear polymers; (iii) crosslinking of native and/or recycled linear polymer compounds to provide crosslinked polymer compounds; (iv) RCD of crosslinked polymer compounds; and/or (v) ROP of cyclic dithioacetal compounds obtained from RCD of crosslinked polymer compounds.

In aspects of the disclosure, the native and/or recycled linear polymer compound has a structure according to Formula I, shown below. The native and/or recycled linear polymer can be a homopolymer, wherein the same dithiol monomer component and the same aldehyde-containing monomer component are used such that each R, each ring A, each X, and each m, for each occurrence, is the same. In other aspects of the disclosure, the native and/or recycled linear polymer can be a copolymer, wherein a dithiol monomer component and an aldehyde-containing monomer component are used such that (i) R, for at least two occurrences is different; (ii) each ring A, for at least two occurrences is different; (iii) each X, for at least two occurrences is different; and/or (iv) each m, for at least two occurrences is different.

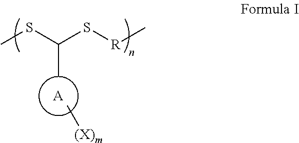

With reference to Formula I, the following substituent recitations can apply:
ring A is an aromatic ring system;
each R independently is selected from aliphatic, aromatic, heteroaliphatic, or any combination thereof; or each R independently can be selected from an R′ group, wherein R′ has a structure according to a Formula A, which is —R^{c}—S—R^{d}—S—R^{c}—, wherein each R^{c} is selected from the groups recited for R, and R^{d} is a group that imparts geometrical flexibility and/or functionality into the linear polymer backbone, such as a group having a Formula B, which is —R^{c}—S[CH₂]_{q}O—Ar—C(Me₂)-Ar—O[CH₂]_{q}S—R^{c}— (which can introduce changes in the geometry of the linear polymer backbone due to the geminal dimethyl group), or a Formula C, which is —R^{c}—S[CH₂]_{q}Y[CH₂]_{q}S—R^{c}— (which can introduce different functionality into the linear polymer backbone, such as by modifying the Y variable), wherein for Formula B, each Ar is an aromatic ring system; for both Formulas B and C, each q independently is an integer selected from 1 to 10, such as 1, 2, 3, 4, 5, 6, 7, 8, 9, or 10; and for Formula C, Y is selected from an amide group, a carbamide group, or a sulfonyl group;
each X independently is selected from an electron-withdrawing group or an electron-donating group; for example, each X independently is selected from aliphatic, aromatic, heteroaliphatic, haloaliphatic, or any combination thereof, or halogen, nitro, cyano, hydroxyl, thiol, or —NH₂;
n is an integer providing a Mₙ ranging from greater than 0 g/mol to 120,000 g/mol, or higher, such as a Mₙ ranging from 5,000 g/mol to 120,000 g/mol or higher, or 10,000 g/mol to 120,000 g/mol or higher. In some aspects of the disclosure, n is an integer selected from at least 2, such as 2 to 200 or higher; and
m is an integer ranging from 0 to 5.

In particular aspects of the disclosure, the following substituent recitations for Formula I can apply:
ring A is an aryl or heteroaryl ring system;
each R independently is selected from alkyl, alkenyl, alkynyl, aryl, heteroaryl, heteroalkyl, heteroalkenyl, heteroalkynyl, or any combination thereof;
each X independently is selected from alkyl, alkenyl, alkynyl, aryl, heteroaryl, heteroalkyl, heteroalkenyl, heteroalkynyl, haloalkyl, haloalkenyl, haloalkynyl, or any combination thereof, or Br, F, I, Cl, nitro, cyano, hydroxyl, thiol, or —NH₂;
n is an integer selected to provide a Mₙ ranging from 10,000 g/mol to 120,000 g/mol; and
m is an integer ranging from 0 to 5, such as 0, 1, 2, 3, 4, or 5.

In yet additional particular aspects of the disclosure, the following substituent recitations for Formula I can apply:
ring A is phenyl, naphthyl, thiophene, furan, pyrrole, or indolyl;
each R independently is selected from C₆-C₁₀ alkyl, ether, thioether, aryl, biaryl, triaryl, -Ph-Z-Ph- (wherein Z is oxygen, sulfur, or NH), —CH₂PhCH₂—, —CH₂C(O)O(CH₂)ᵣO(O)CCH₂— (wherein r is an integer ranging from 1 to 10), —R^{c}—S[CH₂]_{q}O-Ph-C(Me₂)-Ph-O[CH₂]_{q}S—R^{c}— (wherein each R^{c} is C₆-C₁₀ alkyl; ether; thioether; aryl; biaryl; triaryl; -Ph-Z-Ph-, wherein Z is oxygen, sulfur, or NH; —CH₂PhCH₂—, or —CH₂C(O)O(CH₂)ᵣO(O)CCH₂—, wherein r is an integer ranging from 1 to 10; and each q is an integer selected from 1 to 4); or —R^{c}—S[CH₂]_{q}Y[CH₂]_{q}S—R¹— (wherein each R^{c} is C₆-C₁₀ alkyl; ether; thioether; aryl; biaryl; triaryl; -Ph-Z-Ph-, wherein Z is oxygen, sulfur, or NH; —CH₂PhCH₂—, or —CH₂C(O)O(CH₂)ᵣO(O)CCH₂—, wherein r is an integer ranging from 1 to 10; each q is an integer selected from 1 to 4; and Y is carbamide, amide, or sulfonyl);
each X independently is selected from alkyl, alkenyl, alkynyl, aryl, heteroaryl, heteroalkyl, heteroalkenyl, heteroalkynyl, haloalkyl, haloalkenyl, haloalkynyl, or any combination thereof, or Br, F, I, Cl, nitro, cyano, hydroxyl, thiol, or —NH₂;
n is an integer selected to provide a Mₙ ranging from 10,000 g/mol to 120,000 g/mol; and
m is an integer ranging from 0 to 3, such as 0, 1, 2, or 3.

In certain representative aspects of the disclosure, the following substituent recitations can apply for Formula I:
ring A is phenyl, thiophene, furan, or pyrrole;
each R independently is selected from C₆ alkyl, C₈ alkyl, C₁₀ alkyl, —[CH₂]₂O[CH₂]₂—, —[CH₂]₂S[CH₂]₂—, —[CH₂]₂[OCH₂]₂—, —[CH₂]₂[SCH₂]₂—, phenyl, Ph-C(O)OH, biphenyl, triphenyl, -Ph-O-Ph-, -Ph-S-Ph-, —CH₂PhCH₂—, —CH₂C(O)O(CH₂)₄O(O)CCH₂—, —R^{c}—S[CH₂]₂O-Ph-C(Me₂)-Ph-O[CH₂]₂S—R^{c}— (wherein each R^{c} is C₆ alkyl, C₃ alkyl, C₁₀ alkyl, —[CH₂]₂O[CH₂]₂—, —[CH₂]₂S[CH₂]₂—, —[CH₂]₂[OCH₂]₂—, —[CH₂]₂[SCH₂]₂—, phenyl, biphenyl, triphenyl, -Ph-O-Ph-, -Ph-S-Ph-, —CH₂PhCH₂—, or —CH₂C(O)O(CH₂)₄O(O)CCH₂—); or —R^{c}—S[CH₂]₂C(O)NH[CH₂]₃S—R^{c}—, —R^{c}—S[CH₂]₂NHC(O)NH[CH₂]₂S—R^{c}—, or —R^{c}—S[CH₂]₂SO₂[CH₂]₂S—R^{c}— (wherein each R^{c} is C₆ alkyl, C₈ alkyl, C₁₀ alkyl, —[CH₂]₂O[CH₂]₂—, —[CH₂]₂S[CH₂]₂—, —[CH₂]₂[OCH₂]₂—, —[CH₂]₂[SCH₂]₂—, phenyl, biphenyl, triphenyl, -Ph-O-Ph-, -Ph-S-Ph-, —CH₂PhCH₂—, or —CH₂C(O)O(CH₂)₄O(O)CCH₂—); and in some aspects, each R is the same or each R is different;
each X independently is selected from methyl, ethyl, propyl (including isomers thereof), butyl (including isomers thereof), pentyl (including isomers thereof), hexyl (including isomers thereof), phenyl, —CH═CHPh, —OPh-C(O)H, alkoxy, amine, —N(H)C(O)CH₃, —OC(O)R″ (wherein R″ is hydrogen, aliphatic, aromatic, or heteroaliphatic), H₃C[O(CH₂)₂]ₛO— (wherein s is an integer selected from 1 to 10), Br, F, I, Cl, CF₃, nitro, cyano, hydroxyl, thiol, —NH₂, —C(O)OR″ (wherein R″ is hydrogen, aliphatic, aromatic, or heteroaliphatic), or —C(O)H;
n is an integer selected to provide a Mₙ ranging from 10,000 g/mol to 120,000 g/mol; and
m is an integer ranging from 0 to 3, such as 0, 1, 2, or 3.

In some particular aspects of the disclosure, the native and/or recycled linear polymer compound has a structure according to Formula IA, Formula IB, Formula IC, or Formula ID.

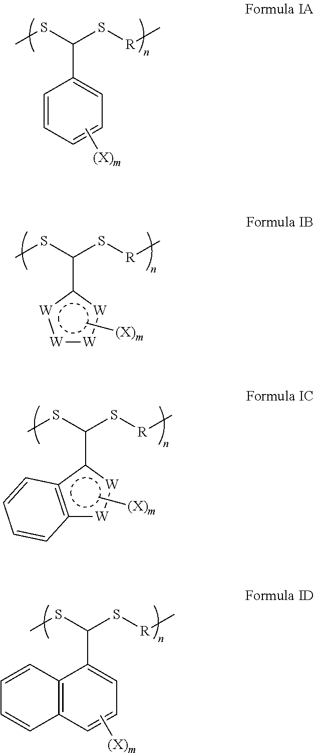

With reference to Formulas IA, IB, IC, and ID, each X and each R independently, for each occurrence, can be as recited herein for Formula I and each of n and m can be as recited for Formula I. With reference to Formulas IB and IC, each W independently is selected from —CH, oxygen, NH (or NX), or S. With reference to Formula IC, X can be bound to the five-membered or 6-membered rings.

In exemplary aspects of the disclosure, the native and/or recycled linear polymer compound can have a structure according to those shown below. With reference to the structures below, n is an integer selected to provide a Mₙ ranging from greater than 0 g/mol to 120,000 g/mol, or higher, such as a Mₙ ranging from 5,000 g/mol to 120,000 g/mol or higher, or 10,000 g/mol to 120,000 g/mol or higher with particular examples having n being an integer ranging from 2 to 200 or higher; R is as recited herein for any of Formulas I, IA, IB, IC, and ID; and R″ is selected from hydrogen, aliphatic, heteroaliphatic, haloaliphatic, or aromatic.

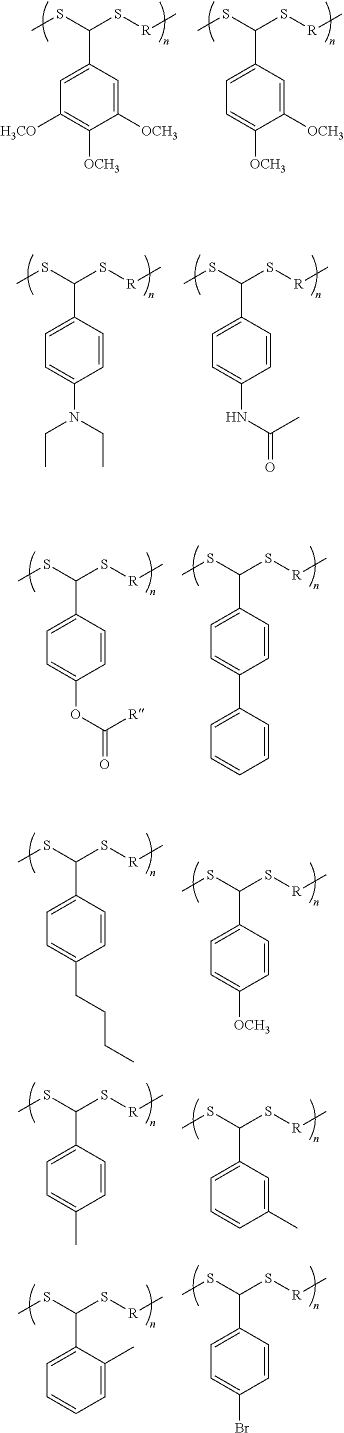

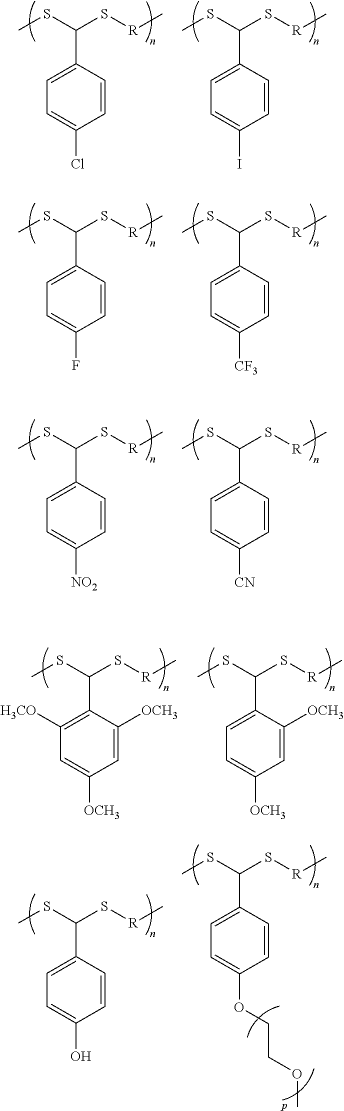

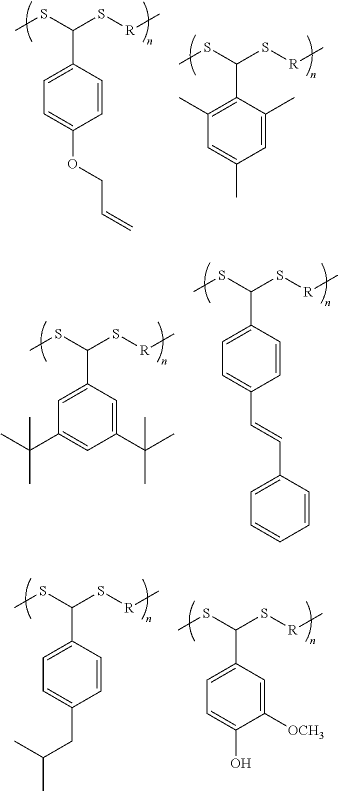

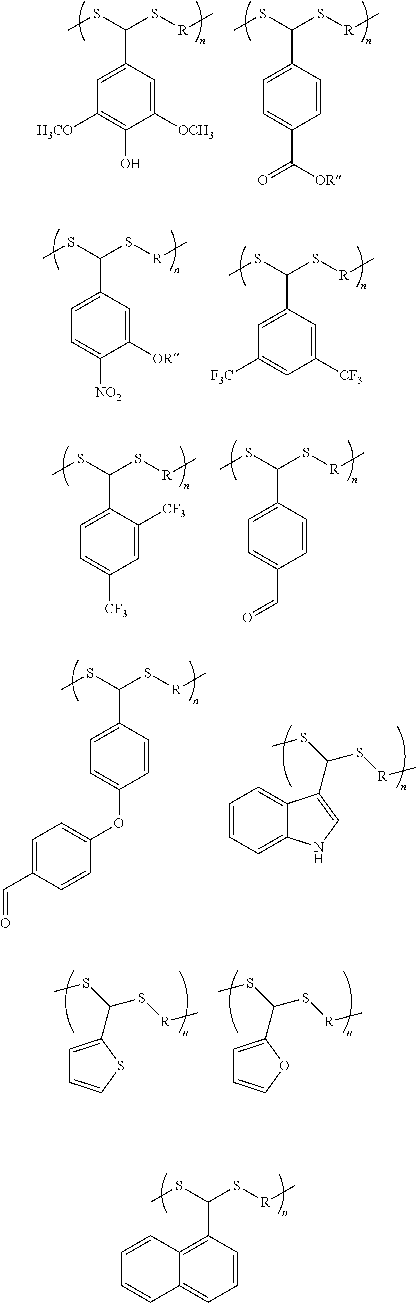

In exemplary aspects of the present disclosure, each R of the above exemplary native and/or recycled linear polymer compounds can independently be selected from the groups shown below.

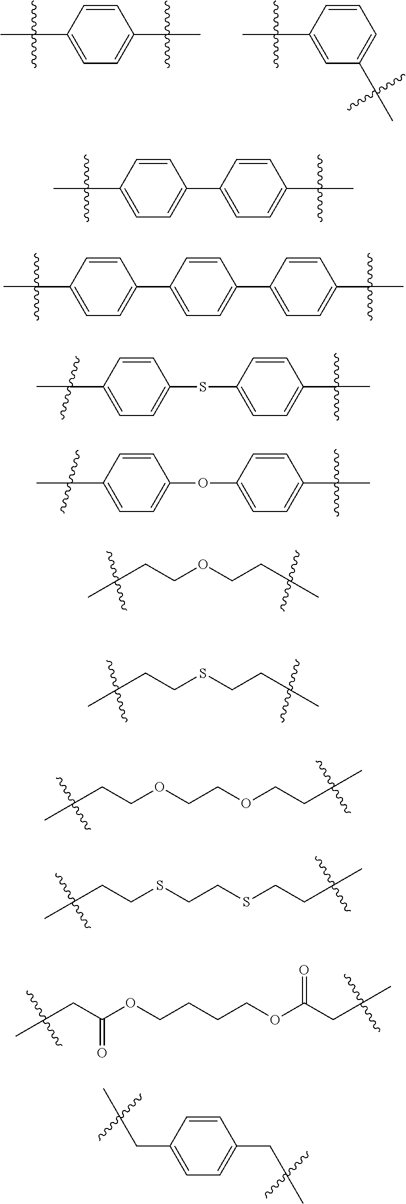

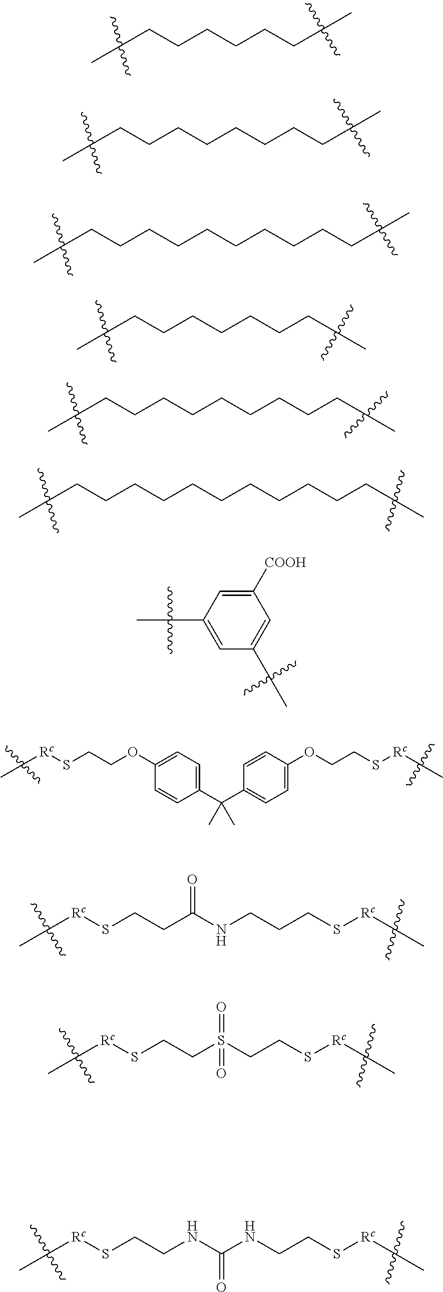

In representative aspects of the disclosure, the native and/or recycled linear polymer has a structure selected from:

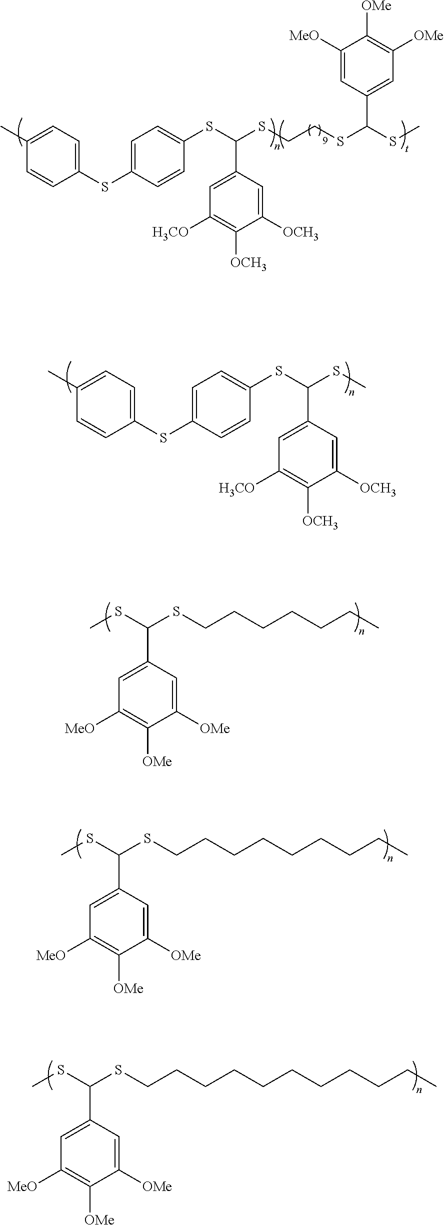

wherein t is an integer selected from values provided herein for n, but where t can be the same or different as n.

In some aspects of the present disclosure, the native and/or recycled linear polymer compounds can be converted to cyclic dithioacetal compounds using the method described herein. In particular aspects, the cyclic dithioacetal compounds have a structure according to Formula II, wherein each of ring A, R, X, and m are as recited herein or illustrated for Formulas I or IA-ID and wherein n′ is an integer selected from 0 to 8, such as 0 to 6, or 0 to 4, or 0 to 2, such as 0, 1, 2, 3, 4, 5, 6, 7, or 9. In some aspects of the disclosure, the cyclic dithioacetal compounds can be formed from ring-closing depolymerization of a native and/or recycled linear homopolymer as described herein, or a native and/or recycled linear copolymer as described herein.

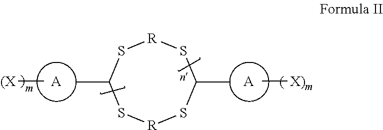

In some aspects of the disclosure, the cyclic dithioacetal compound can have a structure according to Formula IIA, IIB, IIC, or IID. With reference to these formulas, each n′ can be as recited above for Formula II, and each W independently is selected from —CH, oxygen, NH (or NX), or S. With reference to Formula IIC, variable X can be bound to the five-membered or 6-membered rings.

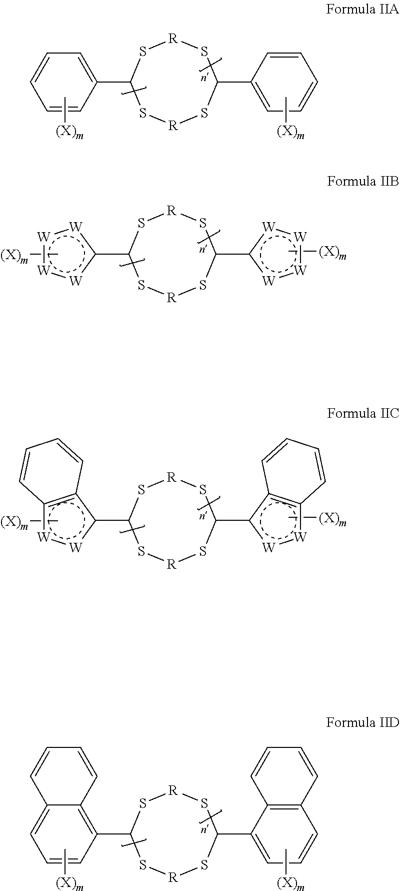

In some aspects of the disclosure, the cyclic dithioacetal compound is selected from any of the following, wherein n is an integer ranging from 2 to 8, R is as recited herein for any of Formulas II, IIA, IIB, IIC, or IID and R″ is selected from hydrogen, aliphatic, heteroaliphatic, haloaliphatic, or aromatic.

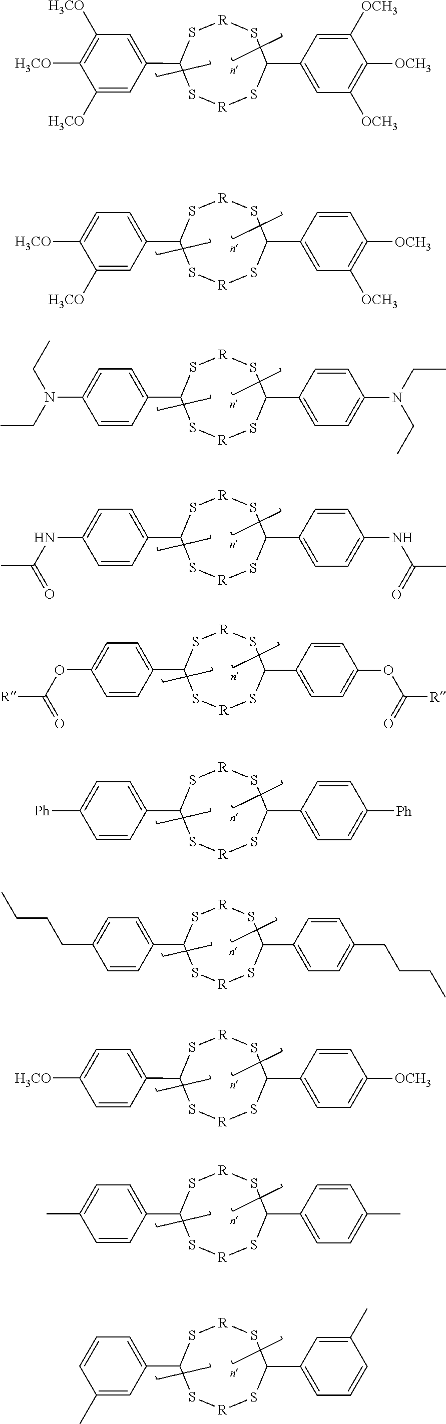

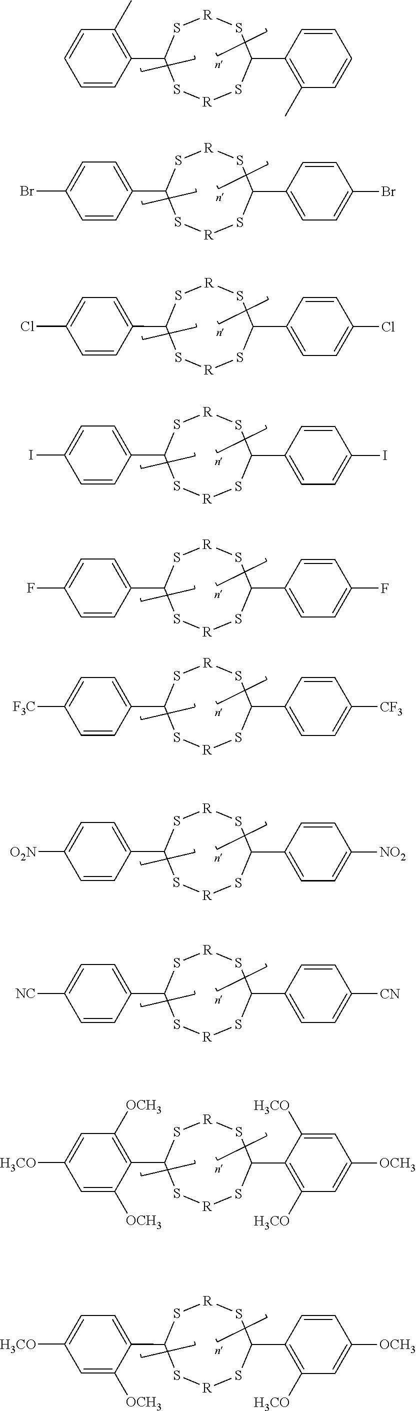

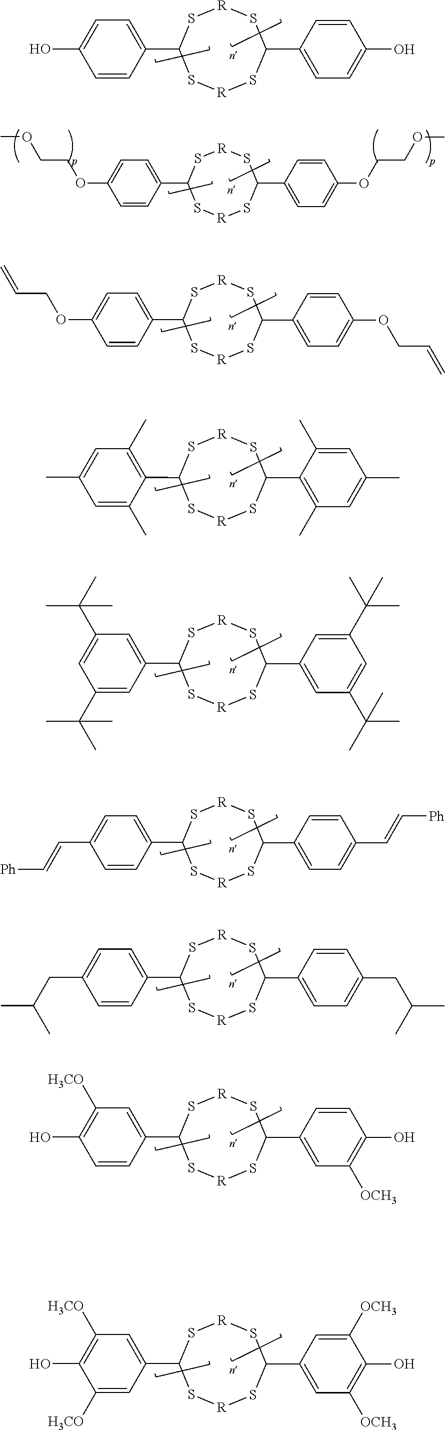

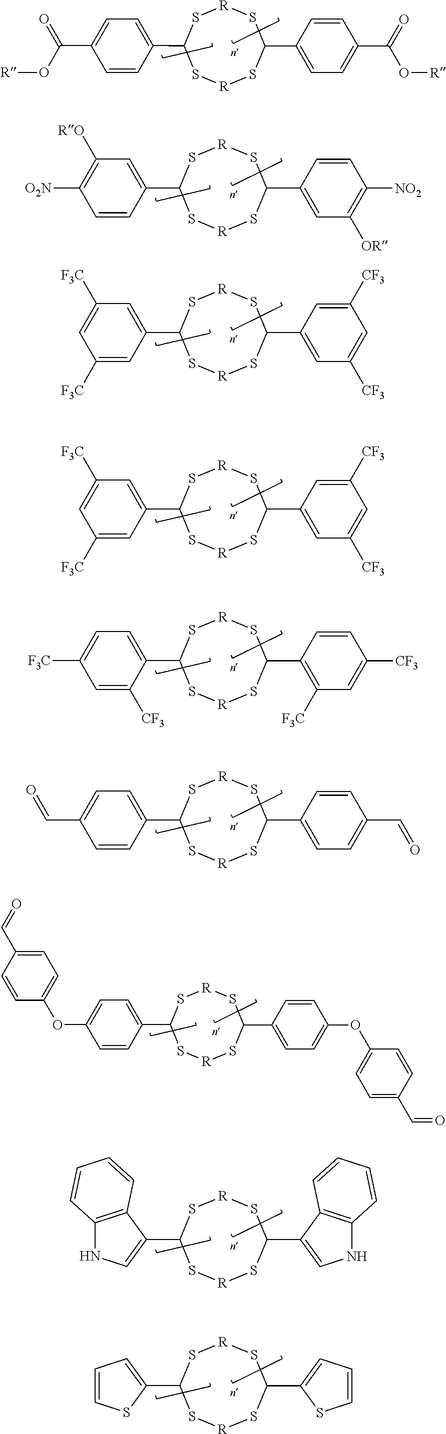

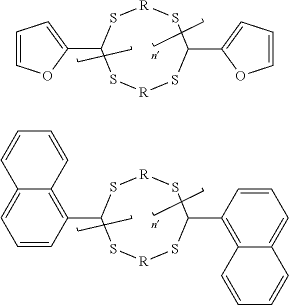

In exemplary aspects of the present disclosure, each R of the above exemplary cyclic dithioacetal compounds can independently be selected from the groups shown below.

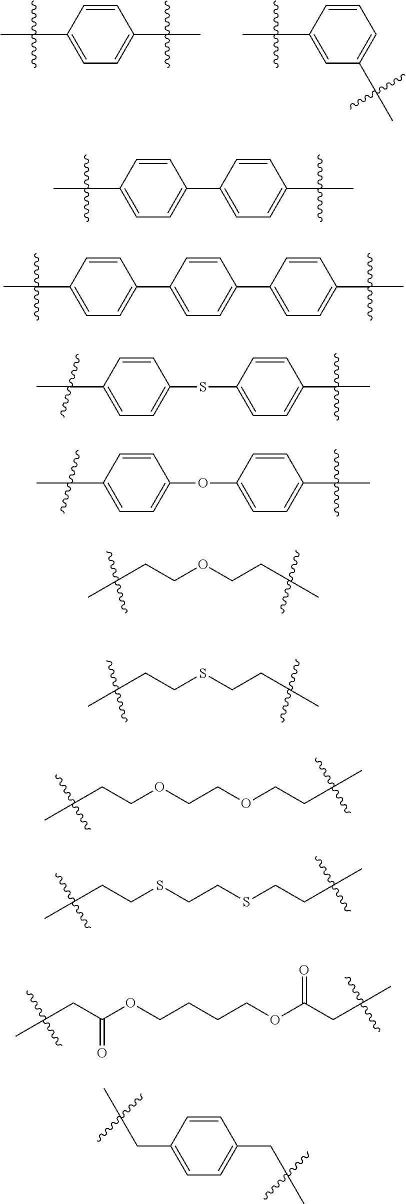

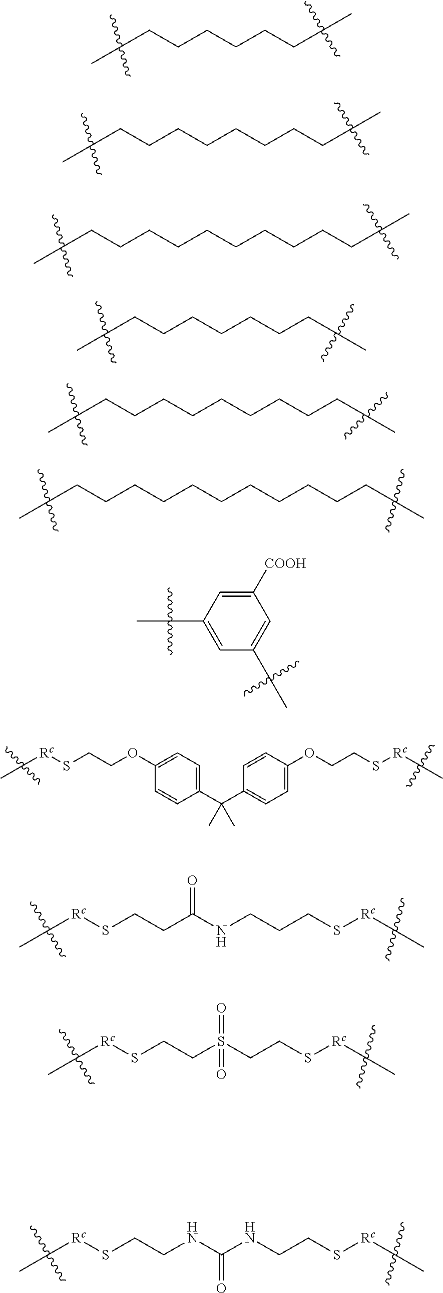

In some representative aspects of the disclosure, the cyclic dithioacetal compound has a structure according to a formula as shown below:

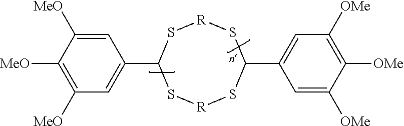

wherein R is (CH₂)₆, (CH₂)₈, (CH₂)₁₀, or -Ph-S-Ph- and n′ is an integer ranging from 1 to 8; or wherein a combination of two different R groups is present and wherein at least one R is -Ph-S-Ph- and at least one R is (CH₂)₆, (CH₂)₈, or (CH₂)₁₀.

The mechanism by which such cyclic dithioacetal compounds can be made is shown in Scheme 1B, provided herein. As shown in Scheme 1B, the cyclic dithioacetal compounds can have a cyclic dimer structure (such as where n′ of Formula II is 0), a cyclic tetramer structure (such as where n′ of Formula II is 1), a cyclic hexamer structure (such as where n′ of Formula II is 2), and so on (such as where n′ of Formula II is 3, 4, 5, 6, etc.). With reference to the structures in Scheme 1B, the curved lines represent the R group of Formula II. As described herein, these cyclic dithioacetal compounds can be used to provide recycled linear polymers having structures according to any of Formula I or IA-ID. In certain aspects of the disclosure, the cyclic dithioacetal compounds exhibit structural aspects that facilitate ED-ROP to corresponding recycled linear polymers. Harnessing this reactivity provides the ability to effect polymerization under conditions unsuitable for typical enthalpy-driven ring-opening polymerization. In some aspects of the disclosure, the cyclic dithioacetal monomers of the present disclosure can be tuned to improve the monomers' solubility in solvents, which can facilitate obtaining improved yields of the recycled polymer under a range of reaction conditions.

Also described herein are dithiol monomer components and aldehyde-containing monomer components that can be used to make the polymers described herein. In some aspects of the disclosure, the dithiol monomer component comprises a chain component that comprises two terminal thiol groups (with one thiol group positioned at each end of the chain). In such aspects, the dithiol monomer can have a structure according to a Formula III, HS—R—SH, wherein the R group can be as recited herein for any of Formulas I or IA-ID.

Exemplary dithiol monomer components are provided below.

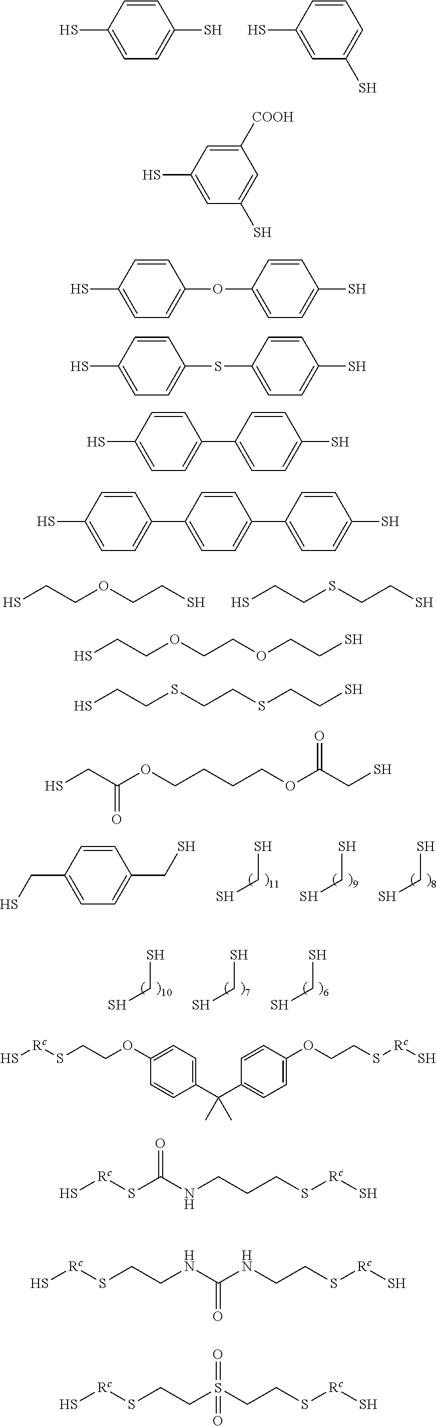

The aldehyde-containing monomer component comprises an aromatic group that is functionalized with at least one aldehyde moiety. The aromatic group of the aldehyde-containing monomer component can further comprise one or more additional substituents. In some aspects of the disclosure, the aldehyde-containing monomer component can have a structure according to Formula IV, wherein each of ring A, X, and m can be as described herein for any of the preceding formulas.

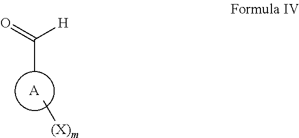

In particular aspects of the disclosure, the aldehyde-containing monomer component can have a structure according to Formulas IVA, IVB, IVC, or IVD, wherein ring A, X, W, and m can be as recited herein for any of the preceding formulas.

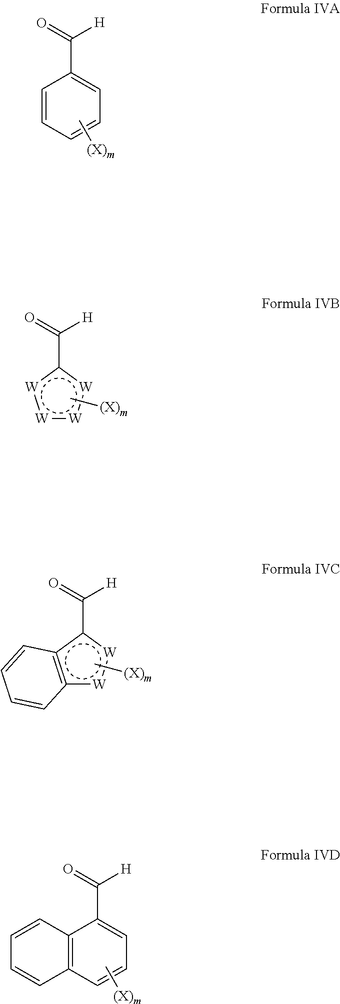

Exemplary aldehyde-containing monomer components are provided below.

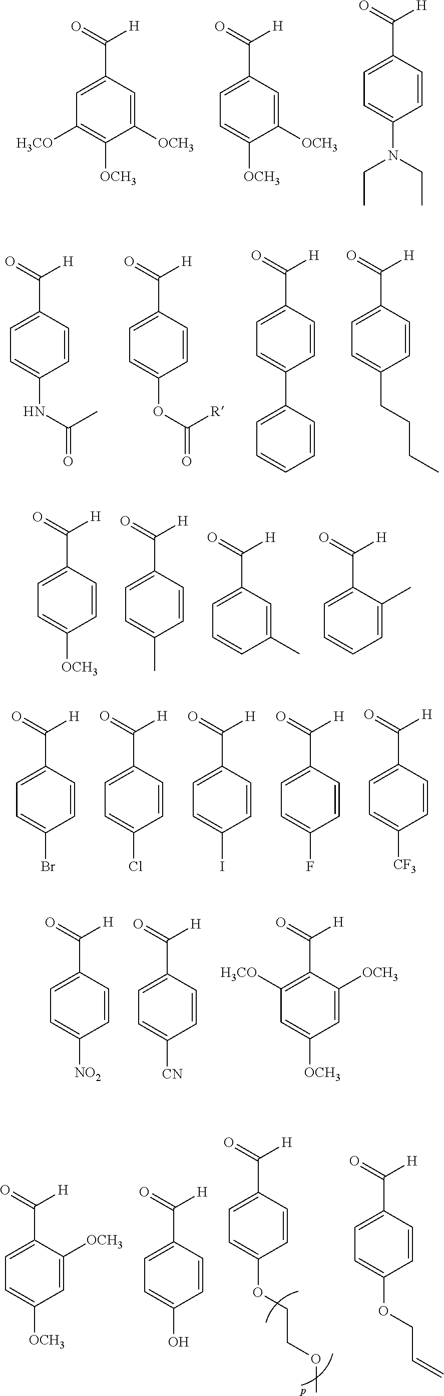

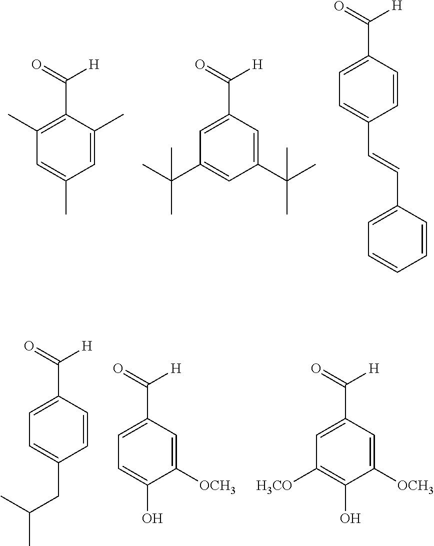

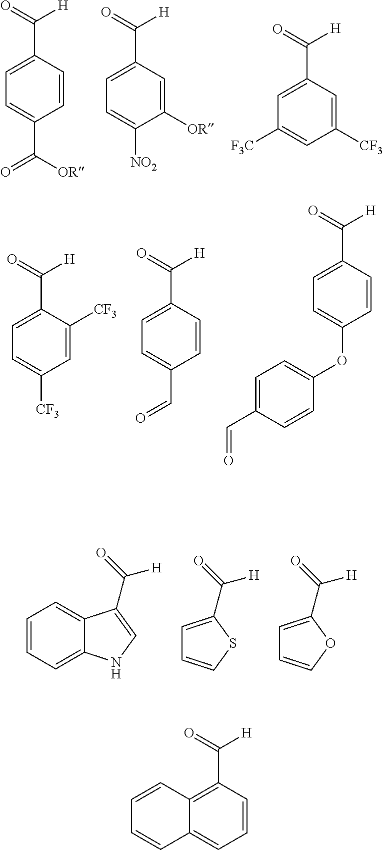

In some aspects of the present disclosure, the native and/or recycled linear polymer compounds can be crosslinked. In some particular aspects, the linear polymer compounds are covalently crosslinked. Crosslinked linear polymer compounds can comprise a covalently bound crosslinker component, such as a bis-aldehyde-containing monomer component that forms bonds with one or more dithiol monomer components during the acid catalyzed reaction between the dithiol monomer component and the aldehyde-containing monomer component. In some representative aspects, the bis-aldehyde-containing monomer component is a ter phthaldehyde compound. Crosslinked polymer compounds of the present disclosure can exhibit different properties from non-crosslinked linear polymer compounds described herein. In some aspects, the crosslinked polymer compounds exhibit different physical properties, such as soft elastomer-like features (e.g., textures similar to silicone rubber). Additionally, the crosslinked polymer compounds can be heat pressed into films. The crosslinked polymer compounds also can swell in solvents without dissolution. In yet further aspects of the disclosure, the crosslinked polymer compounds exhibit increased tensile strength. The crosslinked polymer compounds exhibit good tolerance under reprocessing conditions (e.g., at high temperatures, such as 100° C.) and do not undergo observable depolymerization. The crosslinked polymer compounds therefore are suitably used as vitrimers in applicable applications. In yet additional aspects of the disclosure, the crosslinked polymer compounds exhibit the ability to undergo depolymerization back to the original cyclic dithioacetal compounds and/or cyclic dithioacetal compounds comprising the crosslinker component. The resulting cyclic dithioacetal compounds also exhibit the ability to undergo ring-opening polymerization to provide recycled crosslinked polymers which can have the same or different crosslinked networks compared to the crosslinked polymer compounds obtained prior to RCD and ROP.

Methods for using the dithiol monomer components and the aldehyde-containing monomer components to make native linear polymer compounds are disclosed herein. In an independent embodiment, the native linear polymer is made with a dithiol monomer component other than 1,6-hexanedithiol, 2,2′-(ethane-1,2-diylbis(oxy))bis(ethane-1-thiol), 4,4′-thiodibenzenethiol, butane-1,4-diyl bis(2-mercaptoacetate), butane-1,4-diyl bis(3-mercaptopropanoate), and/or 4,4′-oxydibenzenethiol when the aldehyde-containing monomer component is benzaldehyde; and/or the native linear polymer is made with a dithiol monomer component other than 2,2′-(ethane-1,2-diylbis(oxy))bis(ethane-1-thiol), butane-1,4-diyl bis(2-mercaptoacetate), and/or butane-1,4-diyl bis(3-mercaptopropanoate) if the aldehyde-containing monomer component is 4-methoxybenzaldehyde; and/or the native linear polymer is made with a dithiol monomer component other than 2,2′-(ethane-1,2-diylbis(oxy))bis(ethane-1-thiol) if the aldehyde-containing monomer component is 4-hydroxybenzaldehyde; and/or the native linear polymer is made with a dithiol monomer component other than 2,2′-(ethane-1,2-diylbis(oxy))bis(ethane-1-thiol) if the aldehyde-containing monomer component is 4-(allyloxy)benzaldehyde; and/or the native linear polymer is made with a dithiol monomer component other than 2,2′-(ethane-1,2-diylbis(oxy))bis(ethane-1-thiol) if the aldehyde-containing monomer component is 4-chlorobenzaldehyde; and/or the native linear polymer is made with a dithiol monomer component other than 2,2′-(ethane-1,2-diylbis(oxy))bis(ethane-1-thiol) if the aldehyde-containing monomer component is 4-nitrobenzaldehyde.

### Method of Making and Using

Described herein is a method for making the native linear polymers disclosed herein. The method comprises exposing a dithiol monomer compound to an aldehyde-containing monomer compound in the presence of an acid catalyst. The acid catalyst can be selected from a Lewis acid catalyst, a Brønsted acid catalyst, or a combination thereof. In some aspects of the disclosure, the acid catalyst is a Lewis acid, selected from BF₃, BCl₃, FeCl₃, AlCl₃, GaCl₃, InCl₃, SbCl₃, SbCl₅, BiCl₃, TiCl₄, ZnCl₂, ZrCl₄, SnCl₄, HfCl₄, Zn(OTf)₂, Cu(OTf)₂, Sn(OTf)₂, or any combination thereof. In some aspects of the disclosure, the acid catalyst is a Brønsted acid catalyst selected from p-TsOH (also referred to herein as PTSA), CF₃COOH, 10-camphorsulfonic acid (also referred to herein as CSA), or any combination thereof. In representative aspects of the disclosure, the acid catalyst is Zn(OTf)₂, or PTSA. In some aspects of the disclosure, the method is performed using a suitable solvent; however, other aspects do not require a solvent and the method can be performed neat. The solvent can be selected from acetonitrile, toluene, ethyl acetate, chloroform, dichloromethane, or combinations thereof. The method can be carried out at a reaction temperature ranging from ambient temperature to 80° C., such as ambient temperature to 60° C., or ambient temperature to 50° C. In aspects of the method wherein a solvent is not used, the temperature can be above ambient temperature (e.g., 50° C. to 80° C.).

The dithiol monomer component and the aldehyde-containing monomer component can be used in amounts that provide a 1:1 molar ratio of the two monomer components. In yet some particular aspects, such as when forming a copolymer compound, the ratio of different dithiol monomer components can be modified so as to provide the two different dithiol monomers at molar ratios ranging from 1:9 to 9:1, such as 3:7 to 7:3, or 1:1. In some aspects of the disclosure, the extent of polymerization can be controlled such that polymers of a desired length can be prepared. In some such aspects, the polymerization reaction can be monitored using a suitable technique, such as ¹H NMR and/or gel permeation chromatography (GPC). In particular aspects of the disclosure, termination of polymerization results in producing linear polymer compounds having thiol end groups. Scheme 1 shows an exemplary method for making native linear polymers according to the present disclosure and Scheme 1A provides a proposed exemplary mechanism for the conversion (wherein the curved line is used to represent the R group of the dithiol compound). Also provided is Scheme 1B, which includes proposed exemplary mechanisms for step-wise polymerization and ring-chain equilibrium reactions that can take place using methods described herein (wherein the curved line is used to represent the R group of the dithiol compound).

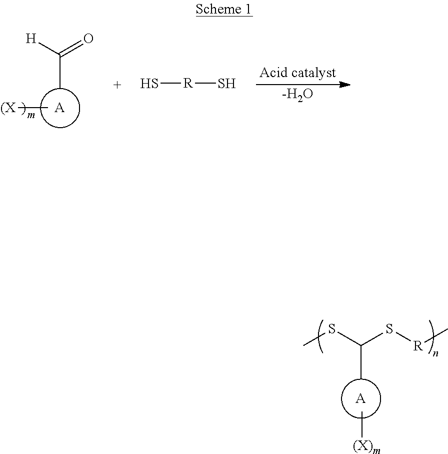

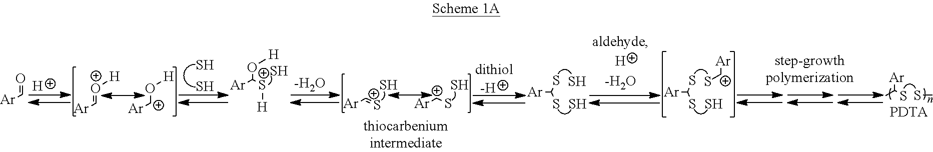

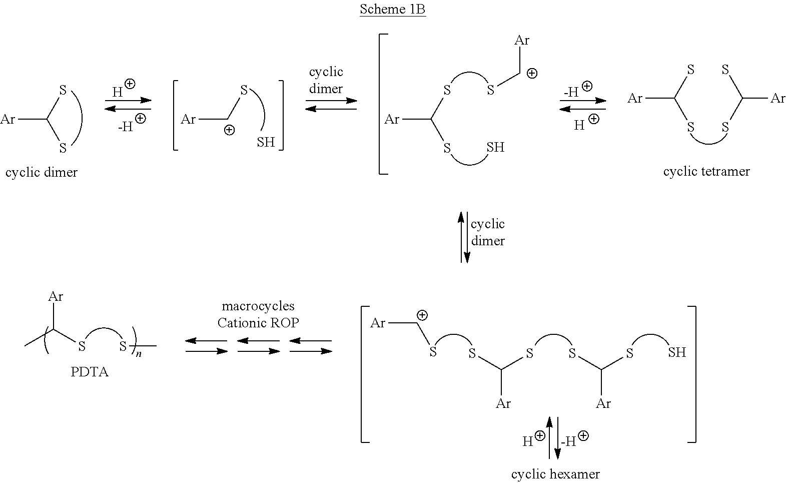

Also disclosed herein is a method for converting a native and/or recycled linear polymer of the present disclosure to a cyclic dithioacetal compound. In such aspects of the disclosure, the method is a chemical recycling method wherein the native and/or recycled linear polymer compound is depolymerized and broken down into one or more of the cyclic dithioacetal compounds of the present disclosure. In some aspects of the disclosure, the depolymerization proceeds via an RCD mechanism. Depolymerization can be achieved by exposing the linear polymer compound to an acid catalyst. The acid catalyst can be the same or different from that used in the polymerization method. In particular aspects, the acid catalyst can be selected from a Lewis acid catalyst, a Brønsted acid catalyst, or a combination thereof. In some aspects of the disclosure, the acid catalyst is a Lewis acid, selected from BF₃, BCl₃, FeCl₃, AlCl₃, GaCl₃, InCl₃, SbCl₃, SbCl₅, BiCl₃, TiCl₄, ZnCl₂, ZrCl₄, SnCl₄, HfCl₄, Zn(OTf)₂, Cu(OTf)₂, Sn(OTf)₂, or any combination thereof. In some aspects of the disclosure, the acid catalyst is a Brønsted acid catalyst, selected from p-TsOH (also referred to herein as PTSA), CF₃COOH, 10-camphorsulfonic acid (also referred to herein as CSA), or any combination thereof. In representative aspects of the disclosure, the acid catalyst is Zn(OTf)₂, or PTSA. Depolymerization can produce different cyclic dithioacetal compounds wherein n′ of Formula II is 0 to 8, such as 0 to 5, or 0 to 3. In some aspects of the disclosure, depolymerization can be controlled by modifying the concentration of the polymer, the temperature used in the method, and/or the solvent used in the method. In some representative aspects, the polymer is used at a concentration ranging from 0.8 wt % to 2.5 wt %, such as 0.9 wt % to 2.5 wt %, or 1 wt % to 2.5 wt %. In some representative aspects, the reaction temperature of the depolymerization is a refluxing temperature of the solvent selected for the method. In some aspects, the reaction temperature ranges from 30° C. to 160° C., such as 40° C. to 150° C., or 40° C. to 145° C. In some representative aspects, the solvent used in the method is modified to facilitate concentration and/or temperature changes. The solvent can be an organic solvent, such as toluene, xylenes, chloroform, dichloromethane, benzene, or combinations thereof.

Also disclosed herein is a method for converting one or more cyclic dithioacetal compounds to a recycled linear polymer. In such aspects of the disclosure, the method comprises exposing one or more cyclic dithioacetal compounds to an acid catalyst to facilitate polymerization to the recycled linear polymer. In particular aspects of the disclosure, the method proceeds via ring-opening polymerization mechanism, with particular aspects involving an ED-ROP mechanism. In particular aspects of the disclosure, a mixture of structurally different (e.g., having different n′ values, ring A structures, R structures, or any combination thereof) cyclic dithioacetal compounds can be used. The acid catalyst used for the method can be the same or different from that used in the polymerization and/or RCD methods described herein. In particular aspects, the acid catalyst can be selected from a Lewis acid catalyst, a Brønsted acid catalyst, or a combination thereof. In some aspects of the disclosure, the acid catalyst is a Lewis acid, selected from BF₃, BCl₃, FeCl₃, AlCl₃, GaCl₃, InCl₃, SbCl₃, SbCl₅, BiCl₃, TiCl₄, ZnCl₂, ZrCl₄, SnCl₄, HfCl₄, Zn(OTf)₂, Cu(OTf)₂, Sn(OTf)₂, or any combination thereof. In some aspects of the disclosure, the acid catalyst is a Brønsted acid catalyst selected from p-TsOH (also referred to herein as PTSA), CF₃COOH, 10-camphorsulfonic acid (also referred to herein as CSA), or any combination thereof. In representative aspects of the disclosure, the acid catalyst is Zn(OTf)₂, CSA, or PTSA. In some aspects of the disclosure, the method used to achieve ED-ROP can comprise using a solution-based polymerization method or a melt-based polymerization method. In some aspects of the disclosure, the method can further comprise exposing the dithiol monomer compound, the aldehyde-containing monomer compound, the acid catalyst, or any combination thereof to an initiator compound. In some such aspects, the initiator compound can be selected from chloromethyl methyl sulfide (CH₃SCH₂Cl), chloromethyl phenyl sulfide (PhSCH₂Cl), chloromethyl trifluoromethyl sulfide (CF₃SCH₂Cl), or any combination thereof.

In solution-based polymerization methods, the cyclic dithioacetal monomer is combined with a solvent. In such a method, temperatures ranging from ambient temperature to 50° C. can be used, such as ambient temperature to 40° C., or ambient temperature to 30° C. The concentration of the cyclic dithioacetal monomer in solution-based methods can range from greater than 0 mol/L to 2 mol/L, such as 0.08 mol/L to 1.5 mol/L, or 0.5 mol/L to 1 mol/L.

In melt-based polymerization methods, the cyclic dithioacetal monomer is used neat, such that no solvent is used. In such a method, temperatures above ambient temperature typically are used, such as temperatures ranging from greater than ambient temperature to 150° C., such as 50° C. to 150° C., or 50° C. to 130° C., or 50° C. to 120° C. In particular aspects of such methods, the polymerization of the cyclic dithioacetal compounds can result in increased molecular weight of polymers relative to a method used to make a native linear polymer described herein. Without being limited to a single theory, it currently is believed that this results from the ability of the cyclic dithioacetal compounds to render a stoichiometric balance/conversion during polymerization, whereas polymerization between a dithiol monomer compound and an aldehyde-containing monomer compound does not always provide such a balance.

Also disclosed herein are methods for making crosslinked polymer compounds. As described herein, such crosslinked polymer compounds can be made by adding a crosslinker component to the method used to make the native linear polymer compound and/or by performing RCD and ROP (e.g., ED-ROP) on linear crosslinked polymers to provide recycled crosslinked polymers. In some aspects of the disclosure, a crosslinked polymer can be made by reacting a dithiol monomer component and an aldehyde-containing monomer component in the presence of an acid catalyst and a crosslinker component (e.g., a ter phthaldehyde compound). In some such aspects, a molar ratio of 10:12:1 (aldehyde-containing monomer component:dithiol monomer component:crosslinker component) can be used. RCD of a crosslinked polymer can be carried out by exposing the crosslinked polymer to an acid catalyst, such as any such acid catalysts described herein, and a solvent and heating the reaction mixture (e.g., heating the reaction mixture by maintaining it at a temperature capable of refluxing the solvent). ROP of the resulting cyclic monomer compounds can be carried out to regenerate recycled crosslinked polymers by exposing the cyclic monomer compounds to an acid catalyst, such as any such acid catalysts described herein, and a solvent. Ambient temperatures can be used in such methods. In some particular aspects of the disclosure, crosslinking of the linear polymer, RCD to the cyclic dithioacetal compounds, and ROP (e.g., ED-ROP) to recycled crosslinked polymers can utilize the same acid catalyst and without requiring addition of the catalyst prior to any RCD and/or ROP (e.g., ED-ROP).

### Overview of Several Embodiments

Disclosed herein are embodiments of a linear polymer having a structure according to Formula I, as described herein, wherein ring A is an aromatic ring system; each R independently is selected from aliphatic, aromatic, heteroaliphatic, or any combination thereof; or each R independently is selected from an R′ group, wherein R′ has a structure according to a Formula A, —R^{c}—S—R^{d}—S—R^{c}—, wherein each R^{c} is selected from the groups recited for R, and R^{d} is a group having a Formula B, —R^{c}—S[CH₂]_{q}O—Ar—C(Me₂)-Ar—O[CH₂]_{q}S—R^{c}—, or a Formula C, —R^{c}—S[CH₂]_{q}Y[CH₂]_{q}S—R^{c}—, wherein for Formula B, each Ar is an aromatic ring system; for both Formulas B and C, each q independently is an integer selected from 1 to 10; and for Formula C, Y is selected from an amide group, a carbamide group, or a sulfonyl group; each X independently is selected from an electron-withdrawing group or an electron-donating group; n is an integer selected to provide an Mₙ ranging from 5,000 g/mol to 120,000 g/mol; and m is an integer ranging from 0 to 5. In independent embodiments, it is provided that (i) if ring A is phenyl and m is 0, then R is not —(CH₂)₆—, —(CH₂CH₂O)₂CH₂CH₂—, -Ph-S-Ph-; (ii) if ring A is phenyl, m is 1, and X is para-methoxy, then R is not —(CH₂)₆—, —(CH₂CH₂O)₂CH₂CH₂—, -Ph-S-Ph-, —CH₂C(O)O(CH₂)₄OC(O)CH₂—, or —(CH₂)₂C(O)O(CH₂)₄OC(O)(CH₂)₂—; (iii) if ring A is phenyl, m is 1, and X is para-OCH₂CH═CH₂, then R is not —(CH₂CH₂O)₂CH₂CH₂—; (iv) if ring A is phenyl, m is 1, and X is para-OH, then R is not —(CH₂CH₂O)₂CH₂CH₂—; (v) if ring A is phenyl, m is 1, and X is para-Cl, then R is not —(CH₂CH₂O)₂CH₂CH₂—; and/or (vi) if ring A is phenyl, m is 1, and X is para-NO₂, then R is not —(CH₂CH₂O)₂CH₂CH₂—.

In any or all of the above embodiments, ring A is an aryl or heteroaryl ring system; each R independently is selected from alkyl, alkenyl, alkynyl, aryl, heteroaryl, heteroalkyl, heteroalkenyl, heteroalkynyl, or any combination thereof; each X independently is selected from alkyl, alkenyl, alkynyl, aryl, heteroaryl, heteroalkyl, heteroalkenyl, heteroalkynyl, haloalkyl, haloalkenyl, haloalkynyl, or any combination thereof, or Br, F, I, Cl, nitro, cyano, hydroxyl, thiol, or —NH₂; n′ is an integer selected to provide an Mₙ ranging from 5,000 g/mol to 120,000 g/mol; and m is an integer ranging from 0 to 5.

In any or all of the above embodiments, ring A is phenyl, naphthyl, thiophene, furan, pyrrole, or indolyl; each R independently is selected from C₆-C₁₀ alkyl; ether; thioether; aryl; biaryl; triaryl; -Ph-Z-Ph- wherein Z is oxygen, sulfur, or NH; —CH₂PhCH₂—; —CH₂C(O)O(CH₂)ᵣO(O)CCH₂—, wherein r is an integer ranging from 1 to 10; or each R independently is —R^{c}—S[CH₂]_{q}O-Ph-C(Me₂)-Ph-O[CH₂]_{q}S—R^{c}— or —R^{c}—S[CH₂]_{q}Y[CH₂]_{q}S—R^{c}— wherein each R^{c} is C₆-C₁₀ alkyl; ether; thioether; aryl; biaryl; triaryl; -Ph-Z-Ph-, wherein Z is oxygen, sulfur, or NH; —CH₂PhCH₂—, or —CH₂C(O)O(CH₂)ᵣO(O)CCH₂—, wherein r is an integer ranging from 1 to 10; and each q is an integer selected from 1 to 4; and Y is carbamide, amide, or sulfonyl; each X independently is selected from alkyl, alkenyl, alkynyl, aryl, heteroaryl, heteroalkyl, heteroalkenyl, heteroalkynyl, haloalkyl, haloalkenyl, haloalkynyl, or any combination thereof, or Br, F, I, Cl, nitro, cyano, hydroxyl, thiol, or —NH₂; n is an integer selected to provide an Mₙ ranging from 10,000 g/mol to 120,000 g/mol; and m is an integer ranging from 0 to 3.

In any or all of the above embodiments, ring A is phenyl, thiophene, furan, or pyrrole; each R independently is selected from C₆ alkyl, C₈ alkyl, C₁₀ alkyl, —[CH₂]₂O[CH₂]₂—, —[CH₂]₂S[CH₂]₂—, —[CH₂]₂[OCH₂]₂—, —[CH₂]₂[SCH₂]₂—, phenyl, Ph-C(O)OH, biphenyl, triphenyl, -Ph-O-Ph-, -Ph-S-Ph-, —CH₂PhCH₂—, —CH₂C(O)O(CH₂)₄O(O)CCH₂—; or each R independently is selected from —R^{c}—S[CH₂]₂O-Ph-C(Me₂)-Ph-O[CH₂]₂S—R^{c}—, —R^{c}—S[CH₂]₂C(O)NH[CH₂]₃S—R^{c}—, —R^{c}—S[CH₂]₂NHC(O)NH[CH₂]₂S—R^{c}—, or —R^{c}—S[CH₂]₂SO₂[CH₂]₂S—R^{c}—, wherein each R^{c} is C₆ alkyl, C₈ alkyl, C₁₀ alkyl, —[CH₂]₂O[CH₂]₂—, —[CH₂]₂S[CH₂]₂—, —[CH₂]₂[OCH₂]₂—, —[CH₂]₂[SCH₂]₂—, phenyl, biphenyl, triphenyl, -Ph-O-Ph-, -Ph-S-Ph-, —CH₂PhCH₂—, or —CH₂C(O)O(CH₂)₄O(O)CCH₂—); each X independently is selected from methyl; ethyl; propyl and isomers thereof; butyl and isomers thereof; pentyl and isomers thereof; hexyl and isomers thereof; phenyl; —CH═CHPh; —OPh-C(O)H; alkoxy; amine; —N(H)C(O)CH₃; —OC(O)R″, wherein R″ is hydrogen, aliphatic, aromatic, or heteroaliphatic; H₃C[O(CH₂)₂]ₛO—, wherein s is an integer selected from 1 to 10; Br; F; I; Cl; CF₃; nitro; cyano; hydroxyl; thiol; —NH₂; —C(O)OR″, wherein R″ is hydrogen, aliphatic, aromatic, or heteroaliphatic; or —C(O)H; n is an integer ranging from 2 to 200; and m is 0, 1, 2, or 3.

In any or all of the above embodiments, the linear polymer has a structure according to Formula IA, IB, IC, or ID as described herein, wherein each W independently is selected from —CH, oxygen, NH, NX, or S.

In any or all of the above embodiments, the linear polymer is a native linear polymer obtained from reacting a dithiol monomer component and an aldehyde-containing monomer compound.

In any or all of the above embodiments, the linear polymer is a recycled linear polymer obtained from ring-closing depolymerization of a native linear polymer to a cyclic dithioacetal compound and subsequent ring-opening polymerization of the cyclic dithioacetal compound.

In any or all of the above embodiments, the linear polymer is selected from exemplary structures provided herein.

In any or all of the above embodiments, R independently is selected from exemplary structures provided herein.

In any or all of the above embodiments, the linear polymer is selected from the following

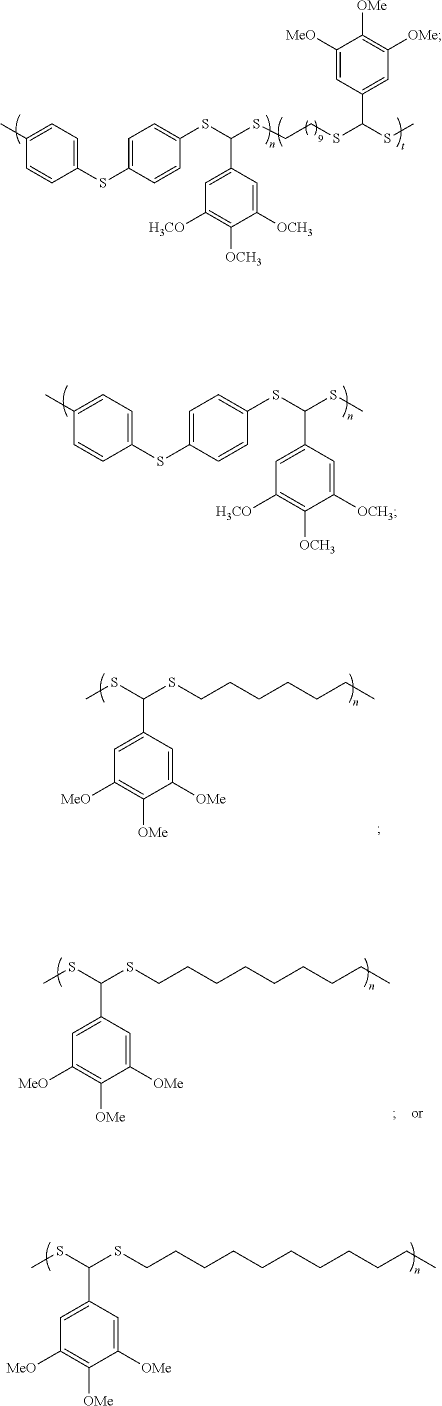

wherein t is selected from values for n and wherein t is the same or different as n.

Also disclosed herein are embodiments of a cyclic dithioacetal compound having a structure according to Formula II as described herein, wherein ring A is an aromatic ring system; each R independently is selected from aliphatic, aromatic, heteroaliphatic, or any combination thereof; or each R independently is selected from an R′ group, wherein R′ has a structure according to a Formula A, —R^{c}—S—R^{d}—S—R^{c}—, wherein each R^{c} is selected from the groups recited for R, and R^{d} is a group having a Formula B, —R^{c}—S[CH₂]_{q}O—Ar—C(Me₂)-Ar—O[CH₂]_{q}S—R^{c}—, or a Formula C, —R^{c}—S[CH₂]_{q}Y[CH₂]_{q}S—R^{c}—, wherein for Formula B, each Ar is an aromatic ring system; for both Formulas B and C, each q independently is an integer selected from 1 to 10; and for Formula C, Y is selected from an amide group, a carbamide group, or a sulfonyl group; each X independently is selected from an electron-withdrawing group or an electron-donating group; n′ is an integer ranging from 1 to 8; and m is an integer ranging from 0 to 5. In independent embodiments, it is provided that (i) if ring A is phenyl and m is 0, then R is not —(CH₂)₆—, —(CH₂CH₂O)₂CH₂CH₂—, -Ph-S-Ph-; (ii) if ring A is phenyl, m is 1, and X is para-methoxy, then R is not —(CH₂)₆—, —(CH₂CH₂O)₂CH₂CH₂—, -Ph-S-Ph-, —CH₂C(O)O(CH₂)₄OC(O)CH₂—, or —(CH₂)₂C(O)O(CH₂)₄OC(O)(CH₂)₂—; (iii) if ring A is phenyl, m is 1, and X is para-OCH₂CH═CH₂, then R is not —(CH₂CH₂O)₂CH₂CH₂—; (iv) if ring A is phenyl, m is 1, and X is para-OH, then R is not —(CH₂CH₂O)₂CH₂CH₂—; (v) if ring A is phenyl, m is 1, and X is para-Cl, then R is not —(CH₂CH₂O)₂CH₂CH₂—; and/or (vi) if ring A is phenyl, m is 1, and X is para-NO₂, then R is not —(CH₂CH₂O)₂CH₂CH₂—.

In any or all of the above embodiments, ring A is an aryl or heteroaryl ring system; each R independently is selected from alkyl, alkenyl, alkynyl, aryl, heteroaryl, heteroalkyl, heteroalkenyl, heteroalkynyl, or any combination thereof; each X independently is selected from alkyl, alkenyl, alkynyl, aryl, heteroaryl, heteroalkyl, heteroalkenyl, heteroalkynyl, haloalkyl, haloalkenyl, haloalkynyl, or any combination thereof, or Br, F, I, Cl, nitro, cyano, hydroxyl, thiol, or —NH₂; n′ is an integer ranging from 1 to 6; and m is an integer ranging from 0 to 5.

In any or all of the above embodiments, ring A is phenyl, naphthyl, thiophene, furan, pyrrole, or indolyl; each R independently is selected from C₆-C₁₀ alkyl; ether; thioether; aryl; biaryl; triaryl; -Ph-Z-Ph- wherein Z is oxygen, sulfur, or NH; —CH₂PhCH₂—; —CH₂C(O)O(CH₂)ᵣO(O)CCH₂—, wherein r is an integer ranging from 1 to 10; or each R independently is —R^{c}—S[CH₂]_{q}O-Ph-C(Me₂)-Ph-O[CH₂]_{q}S—R^{c}— or —R^{c}—S[CH₂]_{q}Y[CH₂]_{q}S—R^{c}— wherein each R^{c} is C₆-C₁₀ alkyl; ether; thioether; aryl; biaryl; triaryl; -Ph-Z-Ph-, wherein Z is oxygen, sulfur, or NH; —CH₂PhCH₂—, or —CH₂C(O)O(CH₂)ᵣO(O)CCH₂—, wherein r is an integer ranging from 1 to 10; and each q is an integer selected from 1 to 4; and Y is carbamide, amide, or sulfonyl; each X independently is selected from alkyl, alkenyl, alkynyl, aryl, heteroaryl, heteroalkyl, heteroalkenyl, heteroalkynyl, haloalkyl, haloalkenyl, haloalkynyl, or any combination thereof, or Br, F, I, Cl, nitro, cyano, hydroxyl, thiol, or —NH₂; n′ is an integer ranging from 1 to 4; and m is an integer ranging from 0 to 3.

In any or all of the above embodiments, ring A is phenyl, thiophene, furan, or pyrrole; each R independently is selected from C₆ alkyl, C₈ alkyl, C₁₀ alkyl, —[CH₂]₂O[CH₂]₂—, —[CH₂]₂S[CH₂]₂—, —[CH₂]₂[OCH₂]₂—, —[CH₂]₂[SCH₂]₂—, phenyl, Ph-C(O)OH, biphenyl, triphenyl, -Ph-O-Ph-, -Ph-S-Ph-, —CH₂PhCH₂—, —CH₂C(O)O(CH₂)₄O(O)CCH₂—; or each R independently is selected from —R^{c}—S[CH₂]₂O-Ph-C(Me₂)-Ph-O[CH₂]₂S—R^{c}—, —R^{c}—S[CH₂]₂C(O)NH[CH₂]₃S—R^{c}—, —R^{c}—S[CH₂]₂NHC(O)NH[CH₂]₂S—R^{c}—, or —R^{c}—S[CH₂]₂SO₂[CH₂]₂S—R^{c}—, wherein each R^{c} is C₆ alkyl, C₈ alkyl, C₁₀ alkyl, —[CH₂]₂O[CH₂]₂—, —[CH₂]₂S[CH₂]₂—, —[CH₂]₂[OCH₂]₂—, —[CH₂]₂[SCH₂]₂—, phenyl, biphenyl, triphenyl, -Ph-O-Ph-, -Ph-S-Ph-, —CH₂PhCH₂—, or —CH₂C(O)O(CH₂)₄O(O)CCH₂—); each X independently is selected from methyl; ethyl; propyl and isomers thereof; butyl and isomers thereof; pentyl and isomers thereof; hexyl and isomers thereof; phenyl; —CH═CHPh; —OPh-C(O)H; alkoxy; amine; —N(H)C(O)CH₃; —OC(O)R″, wherein R″ is hydrogen, aliphatic, aromatic, or heteroaliphatic; H₃C[O(CH₂)₂]ₛO—, wherein s is an integer selected from 1 to 10; Br; F; I; Cl; CF₃; nitro; cyano; hydroxyl; thiol; —NH₂; —C(O)OR″, wherein R″ is hydrogen, aliphatic, aromatic, or heteroaliphatic; or —C(O)H; n′ is an integer ranging from 1 to 4; and m is 0, 1, 2, or 3.

In any or all of the above embodiments, the cyclic dithioacetal compound has a structure according to Formula IIA, IIB, IIC, or IID as described herein, wherein each W independently is selected from —CH, oxygen, NH, NX, or S.

In any or all of the above embodiments, the cyclic dithioacetal compound is selected from exemplary structures provided herein.

In any or all of the above embodiments, each R independently is selected from exemplary structures provided herein.

In any or all of the above embodiments, the cyclic dithioacetal compound has a structure according to a formula

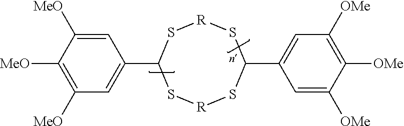

wherein R is (CH₂)₆, (CH₂)₈, or (CH₂)₁₀ and n′ is an integer ranging from 1 to 8; or wherein a combination of two different R groups is present and wherein at least one R is -Ph-S-Ph- and at least one R is (CH₂)₆, (CH₂)₈, or (CH₂)₁₀.

Also disclosed herein are embodiments of a method for making a linear polymer as described herein, wherein the method comprises: exposing a dithiol monomer component and an aldehyde-containing monomer component to an acid catalyst to form a reaction mixture; and exposing the reaction mixture to a reaction temperature; wherein the dithiol monomer component has a structure according to Formula III, HS—R—SH, wherein R is as according any or all of the above embodiments; and the aldehyde-containing monomer component has a structure according to Formula IV, as described herein wherein ring A, X, and m are as recited for any or all of the above embodiments.

In any or all of the above embodiments, the acid catalyst is selected from a Lewis acid catalyst or a Brønsted acid catalyst.

In any or all of the above embodiments, the Lewis acid catalyst is selected from BF₃, BCl₃, FeCl₃, AlCl₃, GaCl₃, InCl₃, SbCl₃, SbCl₅, BiCl₃, TiCl₄, ZnCl₂, ZrCl₄, SnCl₄, HfCl₄, Zn(OTf)₂, Cu(OTf)₂, Sn(OTf)₂, or any combination thereof.

In any or all of the above embodiments, the Brønsted acid catalyst is selected from p-TsOH, CF₃COOH, 10-camphorsulfonic acid, or any combination thereof.

In any or all of the above embodiments, the reaction temperature ranges from ambient temperature to 80° C.

Also disclosed herein are embodiments of a method for making a cyclic dithioacetal compound, comprising: exposing a polymer according to any embodiments described herein to an acid catalyst to provide a reaction mixture; and exposing the reaction mixture to reaction temperature; wherein the cyclic dithioacetal compound has a structure according to Formula II as described herein, wherein substituents for Formula II are as described for any or all of the above embodiments. In independent embodiments, it is provided that (i) if ring A is phenyl and m is 0, then R is not —(CH₂)₆—, —(CH₂CH₂O)₂CH₂CH₂—, -Ph-S-Ph-; (ii) if ring A is phenyl, m is 1, and X is para-methoxy, then R is not —(CH₂)₆—, —(CH₂CH₂O)₂CH₂CH₂—, -Ph-S-Ph-, —CH₂C(O)O(CH₂)₄OC(O)CH₂—, or —(CH₂)₂C(O)O(CH₂)₄OC(O)(CH₂)₂—; (iii) if ring A is phenyl, m is 1, and X is para-OCH₂CH═CH₂, then R is not —(CH₂CH₂O)₂CH₂CH₂—; (iv) if ring A is phenyl, m is 1, and X is para-OH, then R is not —(CH₂CH₂O)₂CH₂CH₂—; (v) if ring A is phenyl, m is 1, and X is para-Cl, then R is not —(CH₂CH₂O)₂CH₂CH₂—; and/or (vi) if ring A is phenyl, m is 1, and X is para-NO₂, then R is not —(CH₂CH₂O)₂CH₂CH₂—.

In any or all of the above embodiments, the method further comprises combining the polymer with a solvent and wherein the reaction temperature is the refluxing temperature of the solvent.

In any or all of the above embodiments, the method further comprises combining the polymer with a solvent and wherein the solvent is a solvent in which the cyclic dithioacetal compound is soluble.

In any or all of the above embodiments, the acid catalyst and reaction temperature promote ring-closing depolymerization of the linear polymer to provide the cyclic dithioacetal compound.

In any or all of the above embodiments, the acid catalyst is a Lewis acid catalyst selected from BF₃, BCl₃, FeCl₃, AlCl₃, GaCl₃, InCl₃, SbCl₃, SbCl₅, BiCl₃, TiCl₄, ZnCl₂, ZrCl₄, SnCl₄, HfCl₄, Zn(OTf)₂, Cu(OTf)₂, Sn(OTf)₂, or any combination thereof; or a Brønsted acid catalyst selected from p-TsOH, CF₃COOH, 10-camphorsulfonic acid, or any combination thereof.

Also disclosed herein are embodiments for making a recycled polymer, comprising exposing a cyclic dithioacetal compound according to any or all of the above embodiments to an acid catalyst to provide the recycled polymer.

In any or all of the above embodiments, the acid catalyst is a Lewis acid catalyst selected from BF₃, BCl₃, FeCl₃, AlCl₃, GaCl₃, InCl₃, SbCl₃, SbCl₅, BiCl₃, TiCl₄, ZnCl₂, ZrCl₄, SnCl₄, HfCl₄, Zn(OTf)₂, Cu(OTf)₂, Sn(OTf)₂, or any combination thereof; or a Brønsted acid catalyst selected from p-TsOH, CF₃COOH, 10-camphorsulfonic acid, or any combination thereof.

In any or all of the above embodiments, the acid catalyst promotes entropy-driven ring-opening polymerization of the cyclic dithioacetal compound to provide the recycled polymer.

Also disclosed herein are embodiments of a method, comprising exposing a linear polymer having a structure according to Formula I to a first acid catalyst to promote ring-closing depolymerization to provide a cyclic dithioacetal compound having a structure according to Formula II; and exposing the cyclic dithioacetal compound to reaction conditions sufficient to promote entropy-driven ring-opening polymerization to provide a recycled linear polymer having a structure according to Formula I and its recited substituents are as recited herein and wherein Formula II and its substituents are as recited herein.

### EXAMPLES

Unless otherwise noted, all starting materials, reagents, and solvents were purchased from commercial sources and used without further purification. NMR spectra were measured for a CDCl₃ solution using a Varian 500 MHz NMR spectrometer. Chemical shift values (6) are reported in parts per million relative to TMS.

Gel permeation chromatography (GPC) was carried out using an Agilent 1260 Infinity GPC system equipped with an autosampler, Agilent 1260 isocratic pump, one Agilent guard column, two Agilent PolyPore columns and a UV (254 nm) detector. The Agilent GPC system was eluted with THE at 35° C. at a rate of 1.0 mL/min and calibrated using monodisperse polystyrene standards. GPC samples were prepared at a concentration of 1.5 mg/mL in HPLC grade THE and the sample solutions were filtered using 0.2 μm PTFE syringe filters prior to injection.

Differential scanning calorimetry (DSC) was performed on a TA Instruments Q20 differential scanning calorimeter equipped with a quench cooler to hold liquid nitrogen. The glass transition temperatures (T_{g}) of polymers were obtained via a heat-cool-heat cycle, where the samples were first heated to 150° C. at 10° C./min, held for 5 min (to erase thermal history), then cooled to −80° C. at 10° C./min, held at −80° C. for 5 min, and reheated to 150° C. at 10° C./min. The data was plotted only from the second heating cycle and T_{g} was calculated as the inflection point.

Thermogravimetric analysis (TGA) was performed on a TA Instruments Q50 under a nitrogen atmosphere, where the samples were heated from room temperature to 600° C. at 10.00° C./min. The decomposition temperature (Td,5%) was taken as the temperature at which a 5% weight loss occurs.

Matrix assisted laser desorption ionization time-of-flight mass spectrometry (MALDI-TOF MS) was performed on a Bruker Microflex spectrometer using 2-(4-hydroxyphenylazo)benzoic acid (HABA) as the matrix.

Tensile testing was conducted on Shimadzu Autograph ASG-X Series at room temperature. The samples are dog-bone shaped with a 12 mm×2.5 mm×1 mm testing area. The strain rate was 1 mm/min. The values reported in the manuscript are an average of three tests.

Rheological testing was performed on Discovery HR-2 Hybrid Rheometer in 8 mm parallel plate configuration. The stress-relaxation experiments were performed with 1 N applied force and 10% strain. The sample was allowed to equilibrate to the testing temperature for 10 min before testing began. The testing gradient started at 150° C. and decreased to room temperature.

### Example 1

### Synthesis of Original Polymer

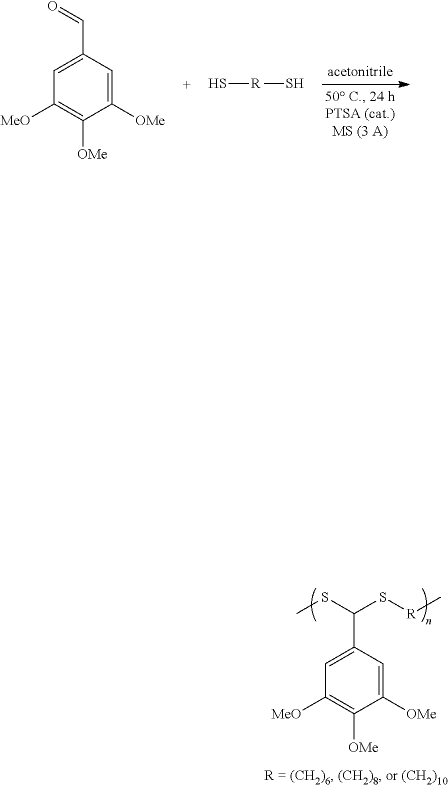

Representative experimental procedure for the synthesis of polymer: An oven-dried scintillation vial equipped with a magnetic stir bar was charged with the dithiol (10.0 mmol), 3,4,5-trimethoxybenzaldehyde (10.0 mmol), and acetonitrile such that the concentration of each of the two monomers were 25 M. The reaction mixture was placed in an oil bath preheated to 50° C. and let all the contents fully dissolved while equilibrating at 50° C. Activated molecular sieves (3 Å) were added and the reaction mixture was degassed. Para-toluenesulfonic acid monohydrate (PTSA) (0.10 mmol) was added, the mixture was purged with N₂ briefly, the vial was capped, and the reaction mixture was stirred for 24 hours at 50° C. The reaction mixture turned from a clear, transparent solution to a white viscous solid within a minute from the PTSA addition. After 24 hours, the reaction was cooled to rt, dissolved in CHCl₃, and partitioned with saturated NaHCO₃ solution. The CHCl₃ layer was separated, and the aqueous layer was extracted two more times with additional CHCl₃, the combined organic layers were washed with water and brine, dried over anhydrous Na₂SO₄, filtered, and concentrated. The polymer was then purified by dissolving in CHCl₃ and precipitating with diethyl ether (×3). The polymer was dried in vacuum. In these examples, trimethoxybenzaldehyde was selected to improve the solubility of the macrocyclic degradation products. Results are provided in Table 1.

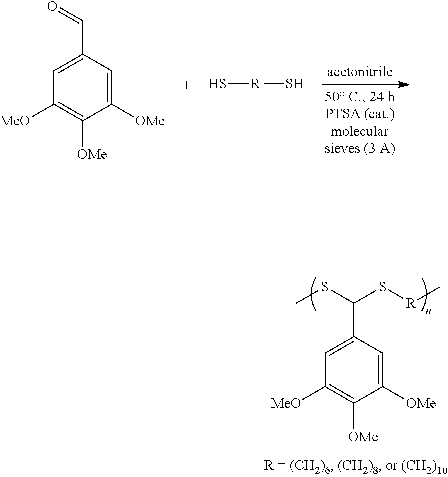

TABLE 1

| | | Conversion^{a} | M_{n, theory}^{b} | | M_{n, GPC}^{d} | | |
|---|---|---|---|---|---|---|---|
| R group | Polymer | (%) | (kg/mol) | Ð_{theory}^{c} | (kg/mol) | Ð_{GPC}^{e} | T_{g}^{f} (° C.) |
| (CH₂)₆ | P1 | 98 | 16 | 1.98 | 38 | 1.92 | 9 |
| (CH₂)₈ | P2 | 99 | 36 | 1.99 | 47 | 1.66 | −1 |
| (CH₂)₁₀ | P3 | 97 | 13 | 1.97 | 53 | 1.56 | −6 |

^{a}Conversion of the benzaldehyde monomer determined by ¹H NMR (CDCl₃) after quenching (triethylamine) an aliquot of the crude reaction mixture at 24 hours.
^{b}Calculated number average molecular weight obtained based on Carothers equation for linear polymers derived from an equimolar mixture of two monomers via step-growth polymerization, where M_{n, theory} = M_{n, repeat unit} × (1/(1 − conversion)).
^{c}Calculated dispersity (Ð) based on Carothers equation, where Ð_{theory} = 1 + conversion.
^{d,e}Number average molecular weights Mₙ and Ð (M_{w}/Mₙ) determined by GPC in THF relative to polystyrene standards at 35° C.
^{f}Glass transition temperature (T_{g}) recorded from the second heating scan by DSC in a heat-cool-heat cycle.

The moderately high yields (63-77%) despite high conversions (>97%) should be primarily because of the loss of the polymer during liquid-liquid extraction as well as the dissolution of the polymer in ether to some extent over three subsequent purification steps. The number average molecular weight (Mₙ), dispersity (Ð=M_{w}/Mₙ), and glass transition temperature (T_{g}) for each polymer are provided above in Table 1. The polymers obtained were odorless, transparent, and soft with glass transition temperatures (T_{g}) ranging from 9 to −6° C. It was observed that the dispersities were close to 2.0 when the polymers were left unpurified (e.g., in presence of the PTSA) at room temperature with a simultaneous increase in molecular weight of the polymers.

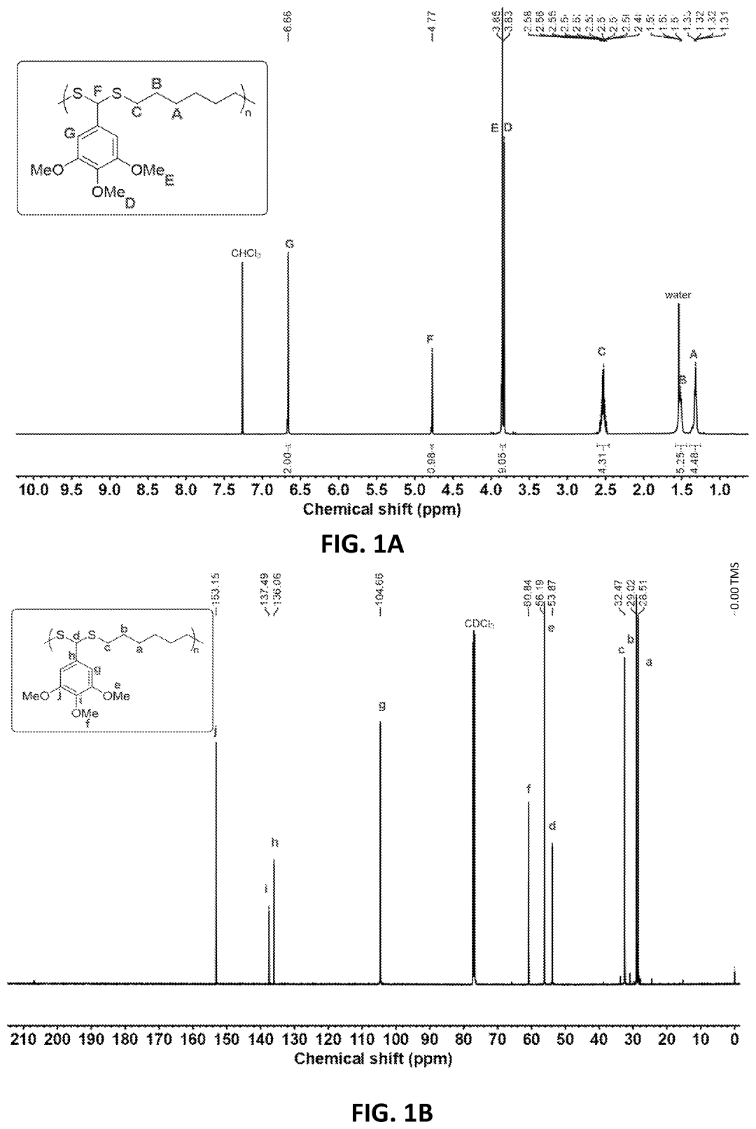

Polymer P1: ¹H NMR (500 MHz, CDCl₃) δ 6.66 (s, 2H), 4.77 (s, 1H), 3.85 (s, 6H), 3.83 (s, 3H), 2.61-2.42 (m, 4H), 1.55-1.47 (m, 4H), 1.39-1.23 (m, 4H). ¹³C{1H} NMR (126 MHz, CDCl₃) δ 153.1, 137.5, 136.1, 104.7, 60.8, 56.2, 53.9, 32.5, 29.0, 28.5. % Yield=63. M_{n, GPC}=38:056 g/mol, Ð=1.92. T_{g}=9° C. T_{d,5%}=272° C. See FIGS. 1A and 1B for the ¹H NMR and ¹³C{1H} NMR, respectively.

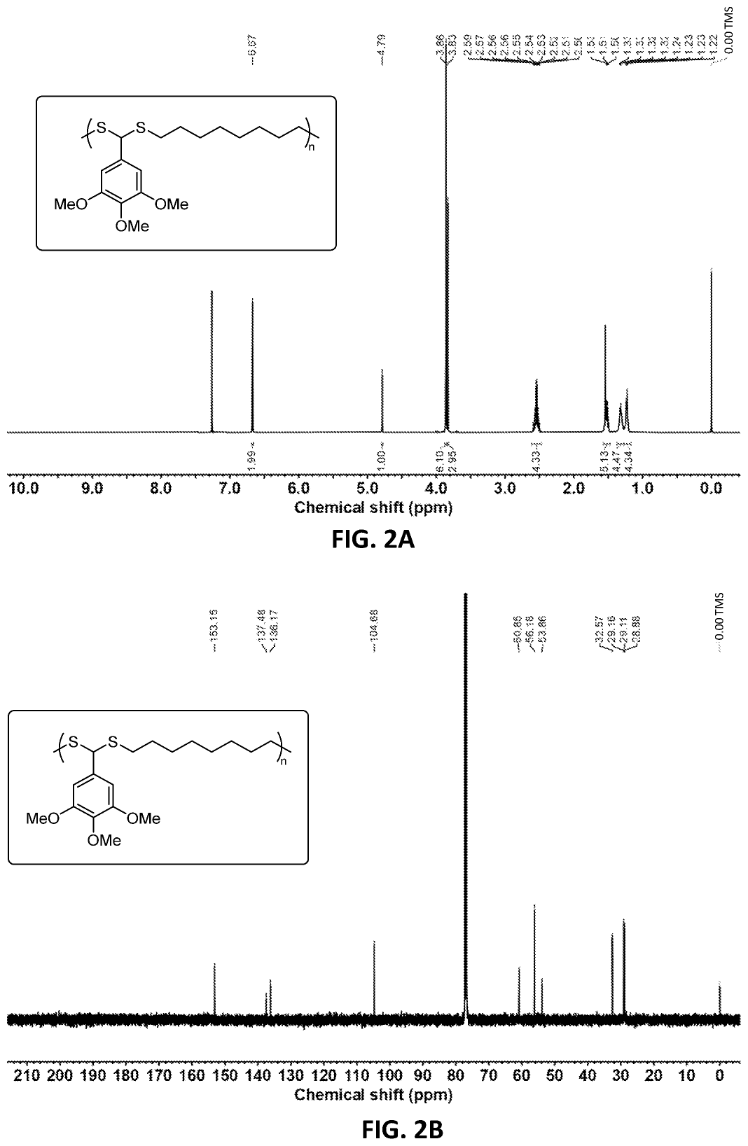

Polymer P2: ¹H NMR (500 MHz, CDCl₃) δ 6.67 (s, 2H), 4.79 (s, 1H), 3.86 (s, 6H), 3.83 (s, 3H), 2.61-2.45 (m, 4H), 1.64-1.43 (m, 4H), 1.36-1.28 (m, 4H), 1.27-1.17 (m, 4H). ¹³C{1H} NMR (126 MHz, CDCl₃) δ 153.1, 137.5, 136.2, 104.7, 60.8, 56.2, 53.9, 32.6, 29.2, 29.1, 28.9. % Yield=77. M_{n, GPC}=46,736 g/mol, Ð=1.66. T_{g}=−1° C. T_{d,5%}=285° C. See FIGS. 2A and 2B for the ¹H NMR and ¹³C{1H} NMR, respectively.

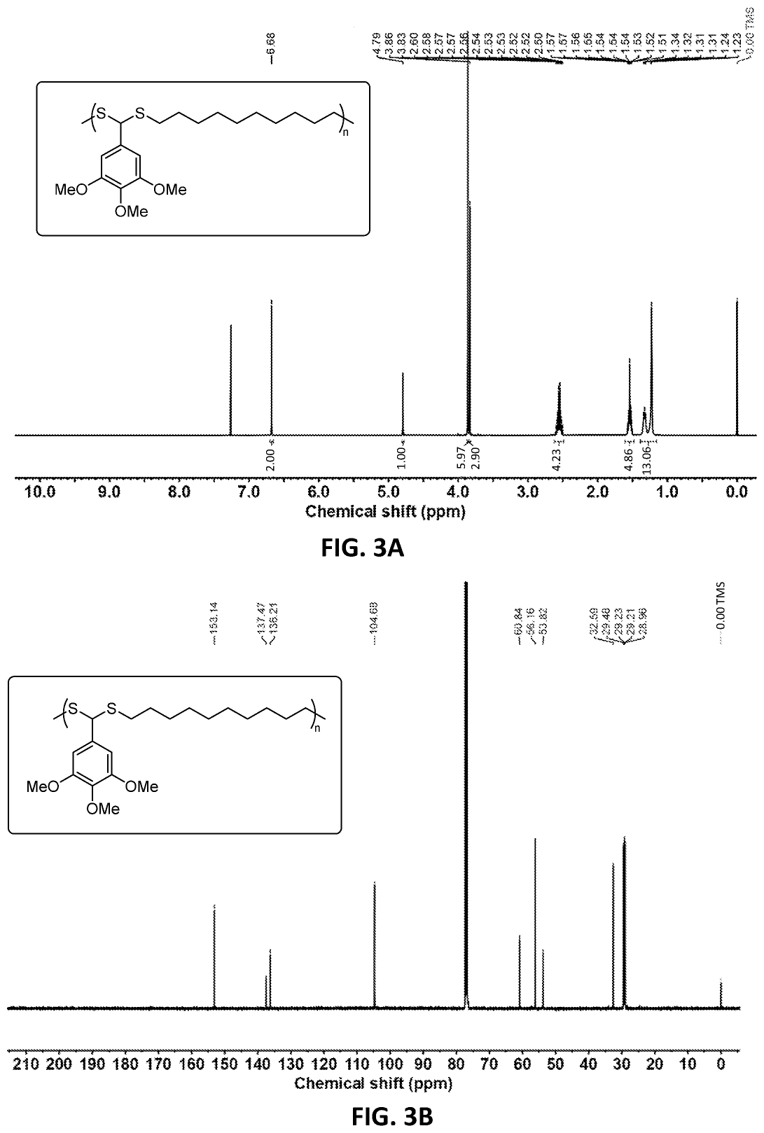

Polymer P3: ¹H NMR (500 MHz, CDCl₃) δ 6.68 (s, 2H), 4.79 (s, 1H), 3.86 (s, 6H), 3.83 (s, 3H), 2.65-2.39 (m, 4H), 1.62-1.45 (m, 4H), 1.42-1.29 (m, 4H), 1.29-1.14 (m, 8H). ¹³C{1H} NMR (126 MHz, CDCl₃) δ 153.1, 137.5, 136.2, 104.7, 60.8, 56.2, 53.8, 32.6, 29.5, 29.23, 29.21, 29.0. % Yield=68. M_{n, GPC}=53,434 g/mol, Ð=1.56. T_{g}=−6° C. T_{d,5%}=270° C. See FIGS. 3A and 3B for the ¹H NMR and ¹³C{1H}NMR, respectively.

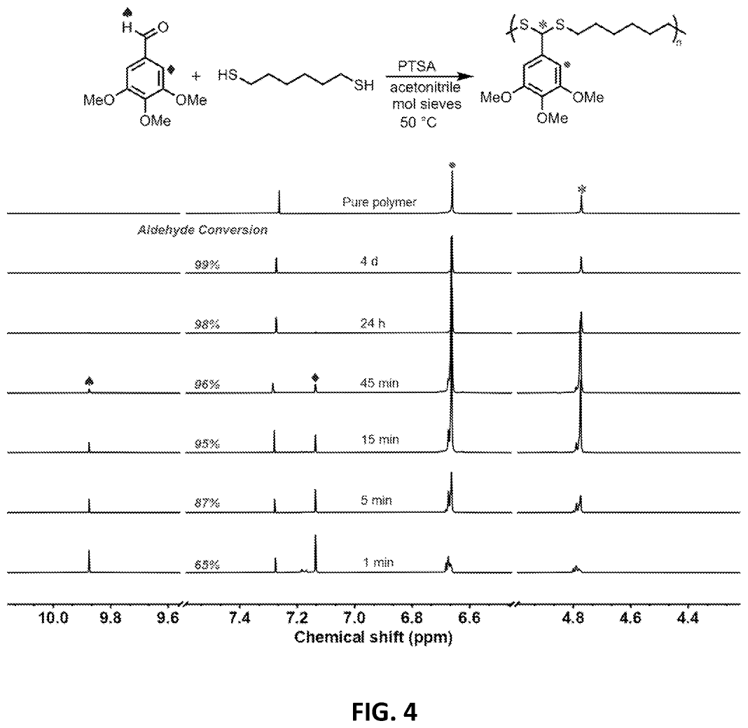

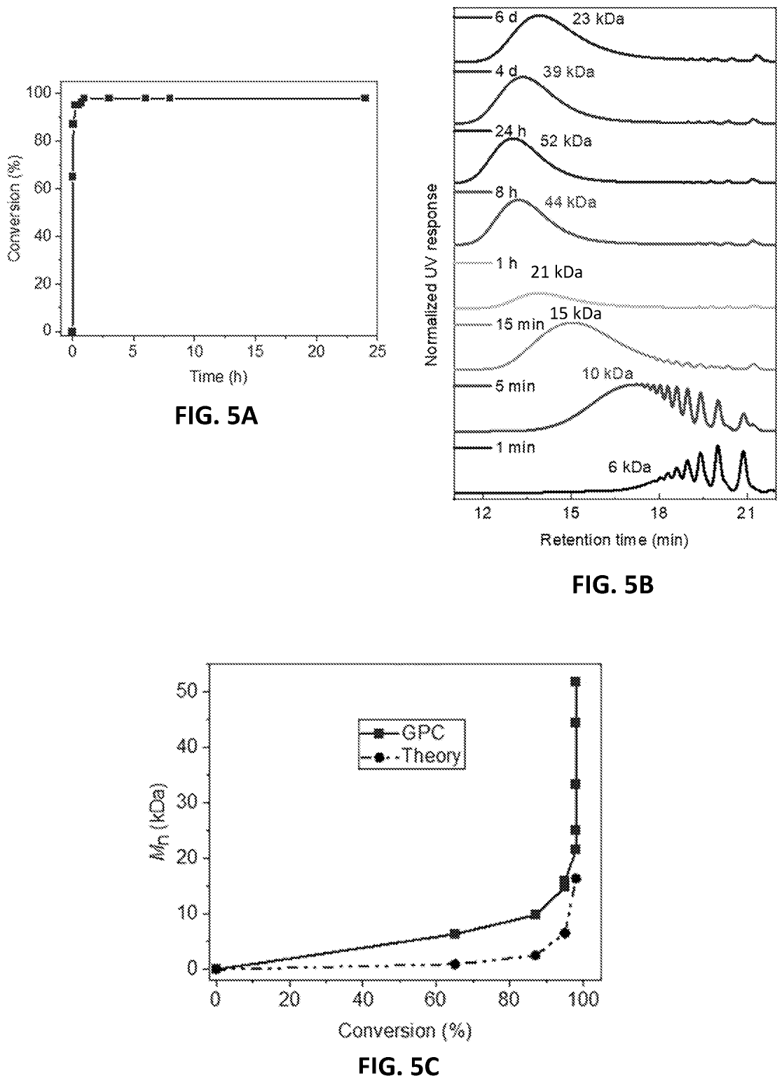

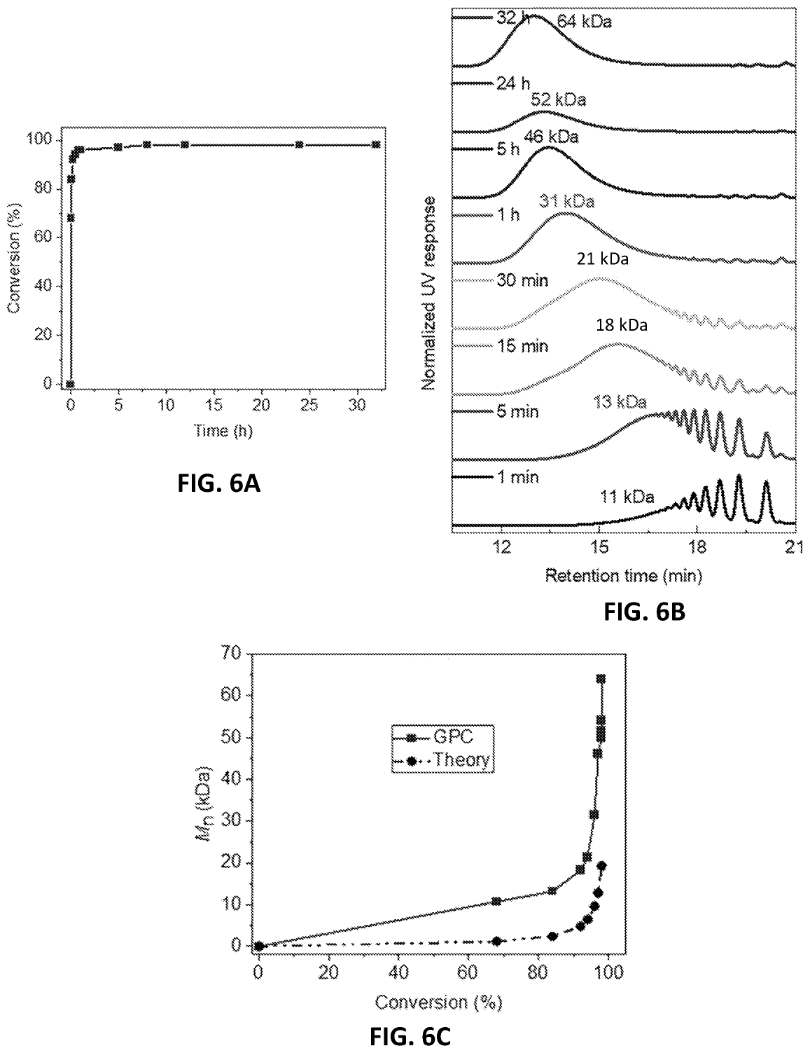

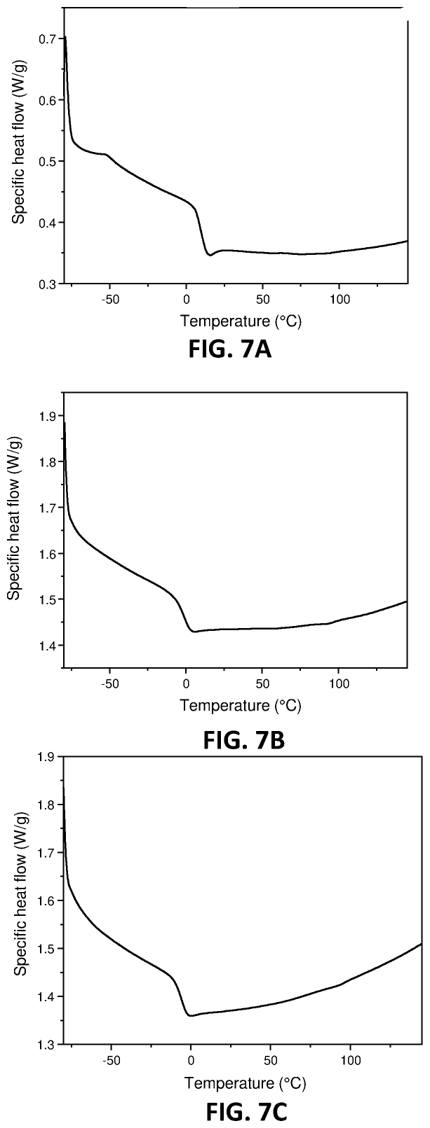

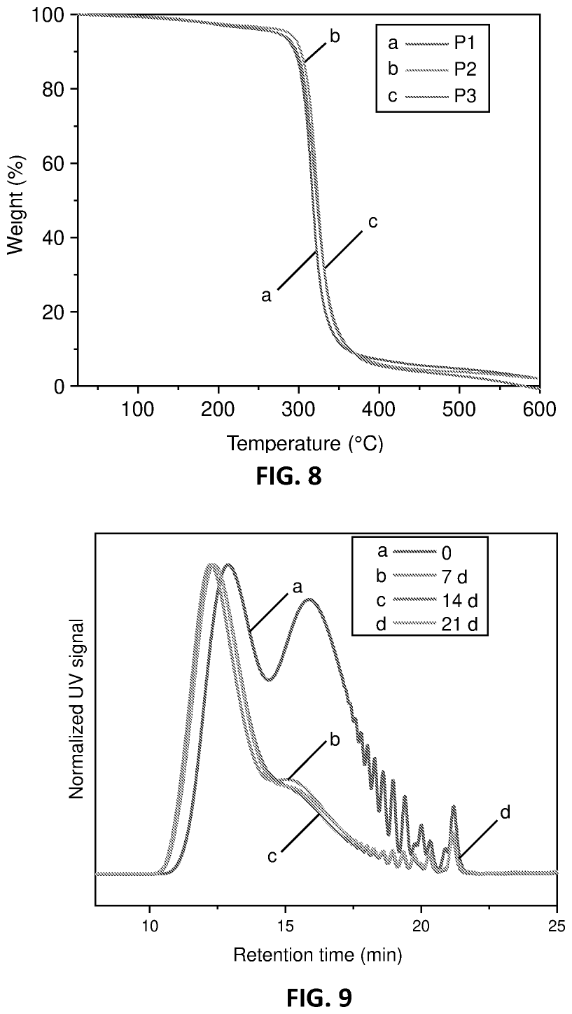

Polymerization was monitored via ¹H NMR and gel permeation chromatography (GPC) over time. Results showing reaction progress between 3,4,5-trimethoxybenzaldehyde and 1,6-hexanedithiol to form P1 are shown in FIGS. 4 and 5A-5C. With reference to FIG. 4, the topmost spectrum corresponds to the purified polymer, while all the other spectra appear underneath are unpurified reaction mixtures quenched with triethylamine at the indicated times. Results showing reaction progress in forming P3 are shown in FIGS. 6A-6C. Tabulated data are provided in Tables 2A-2C. The amounts of macrocycles present in P1-P3 crude polymers were less than 5% and they varied in the order P1>P2>P3. FIGS. 7A-7C show the DSC curves for P1-P3, respectively, and FIG. 8 shows the TGA curves of the three polymers.

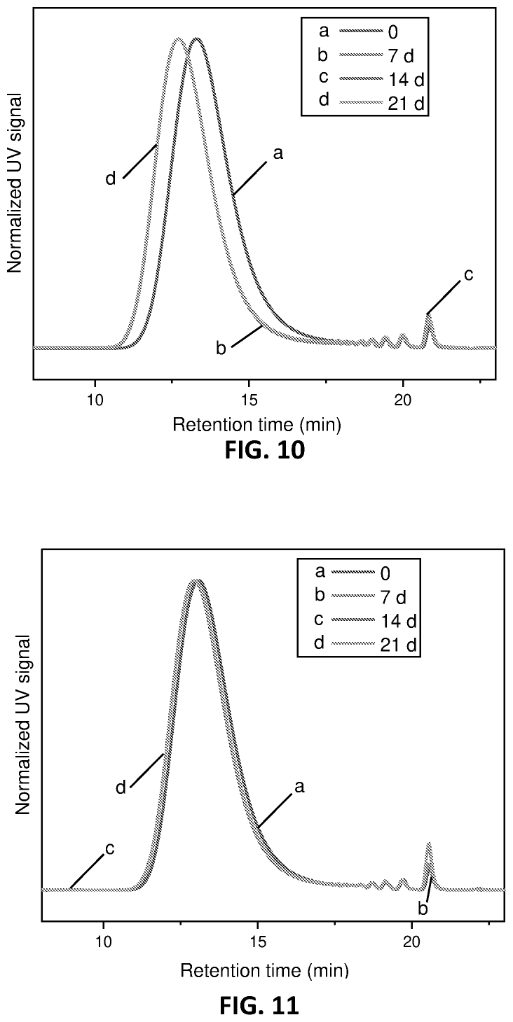

The Ð of the polymers P1-P3 were all close to 2.0 when they were left unpurified (e.g., in presence of PTSA), while showing an increase in molecular weight at room temperature (rt) over the course of weeks (see FIGS. 9-11 and Tables 2A-2C). The lower Ð for purified polymers P2 and P3 as shown in Table 1 possibly came from rapid RCD and ROP (e.g., ED-ROP) equilibrium as the polymers were diluted and concentrated during purification without enough time to randomize.

TABLE 2A
The GPC (THF) monitoring of change in molecular weights
of the polymer (P1) over time when left unpurified.

| Time (days) | M_{n, GPC} (g/mol) | Ð |
|---|---|---|
| 0 | 108,288 | 1.47 |
| | 9,809 | 1.50 |
| 7 | 161,034 | 1.57 |
| | 11,680 | 1.59 |
| 14 | 149,002 | 1.62 |
| | 11,162 | 1.54 |
| 21 | 127,889 | 1.70 |
| | 9,904 | 1.51 |

TABLE 2B
The GPC (THF) monitoring of change in molecular weights
of the polymer (P2) over time when left unpurified.

| Time (days) | M_{n, GPC} (g/mol) | Ð |
|---|---|---|
| 0 | 46,656 | 2.29 |
| 7 | 77,095 | 2.14 |
| 14 | 75,427 | 2.19 |
| 21 | 77,961 | 2.13 |

TABLE 2C
The GPC (THF) monitoring of change in molecular weights
of the polymer (P3) over time when left unpurified.

| Time (days) | M_{n, GPC} (g/mol) | Ð |
|---|---|---|
| 0 | 62,954 | 1.96 |
| 7 | 70,306 | 1.96 |
| 14 | 69,191 | 1.99 |
| 21 | 69,498 | 1.96 |

In some examples, 1,4-butanedithiol and 1,5-pentanedithiol were used as the dithiol precursor; however, both furnished corresponding cyclic dimers almost exclusively over polymerization. In some examples, the Lewis acid zinc(II) triflate was employed in place of PTSA for native linear polymer synthesis and such examples provided comparable results under otherwise same conditions.

### Example 2

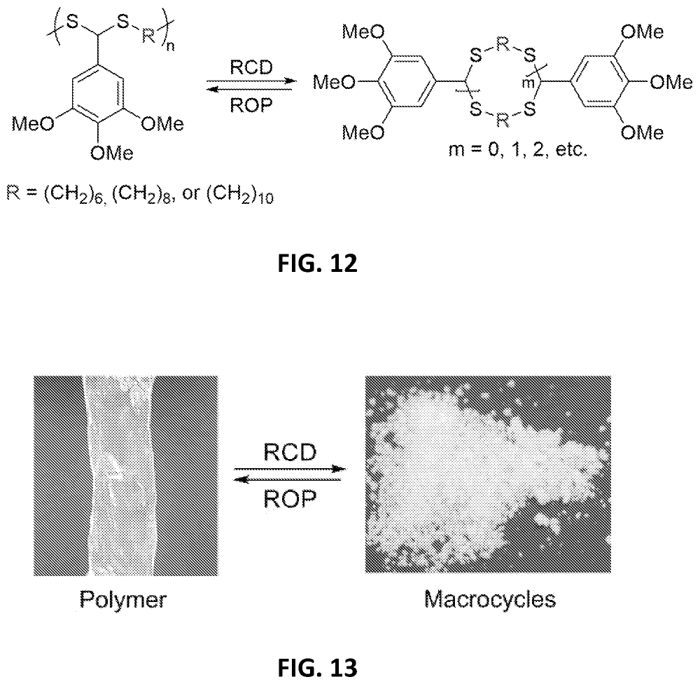

In this example, the native linear polymers P1-P3 were subjected to depolymerization at a concentration of 1.5 wt. % ([dithioacetal]˜35 mmol/L) with zinc(II) triflate (0.3 wt. %) as the catalyst in refluxing toluene overnight (FIG. 12).

### Ring-Closing Depolymerization (RCD)

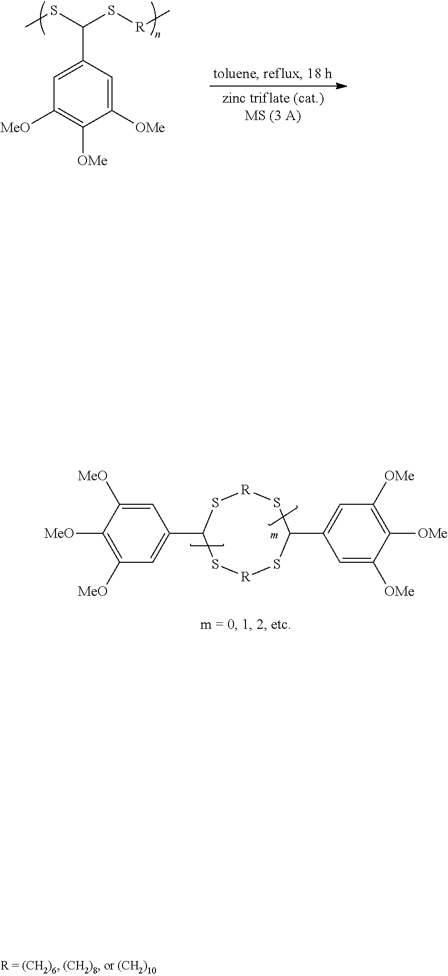

Representative experimental procedure for RCD: An oven-dried round bottomed flask equipped with a magnetic stir bar was charged with the purified polymer (1.5 wt %), dry toluene, zinc triflate (0.3 wt %), and activated molecular sieves (3 Å). The polymer was fully dissolved. The reaction mixture was then degassed and heated to reflux under a continuous nitrogen atmosphere overnight. The reaction was cooled to rt, filtered to remove the catalyst and molecular sieves, and concentrated to remove toluene. The resulting white solid was dissolved in CHCl₃ and partitioned with a large amount of water (to quench the remaining catalyst). The aqueous layer was extracted with CHCl₃ (3×). The combined organic layers were washed with water and brine, then dried over Na₂SO₄, filtered, and concentrated. A mixture of dithioacetal macrocycles was obtained as a white powder that was dried in vacuum. The average yields (by weight) for all three polymers were ca. 85%.

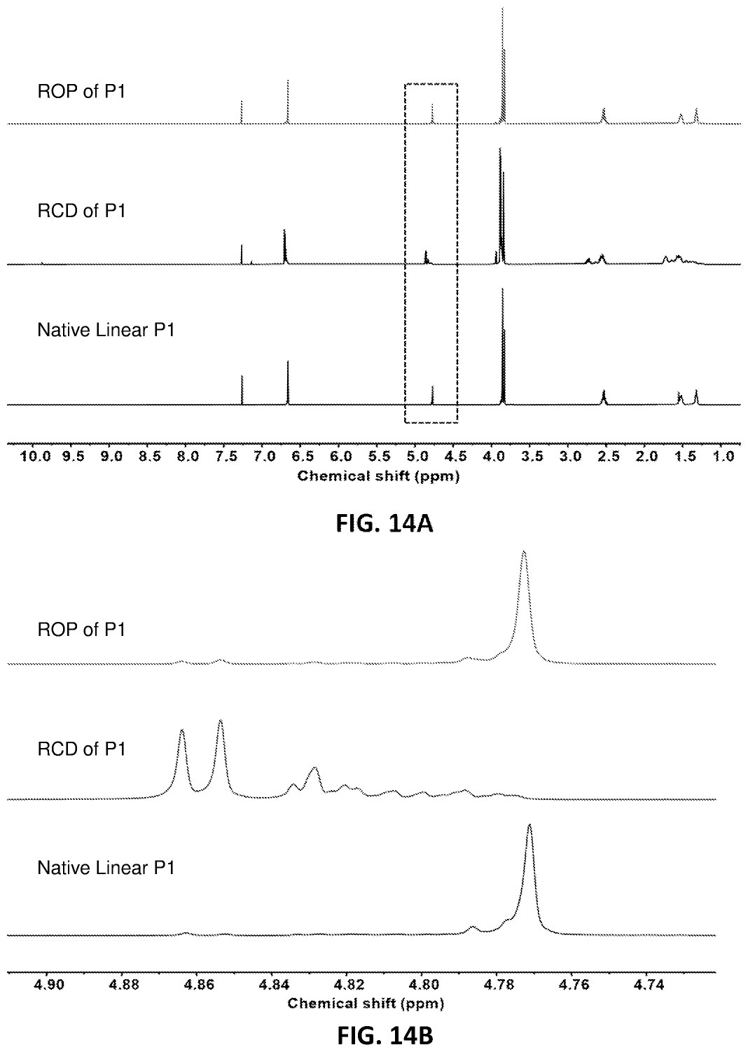

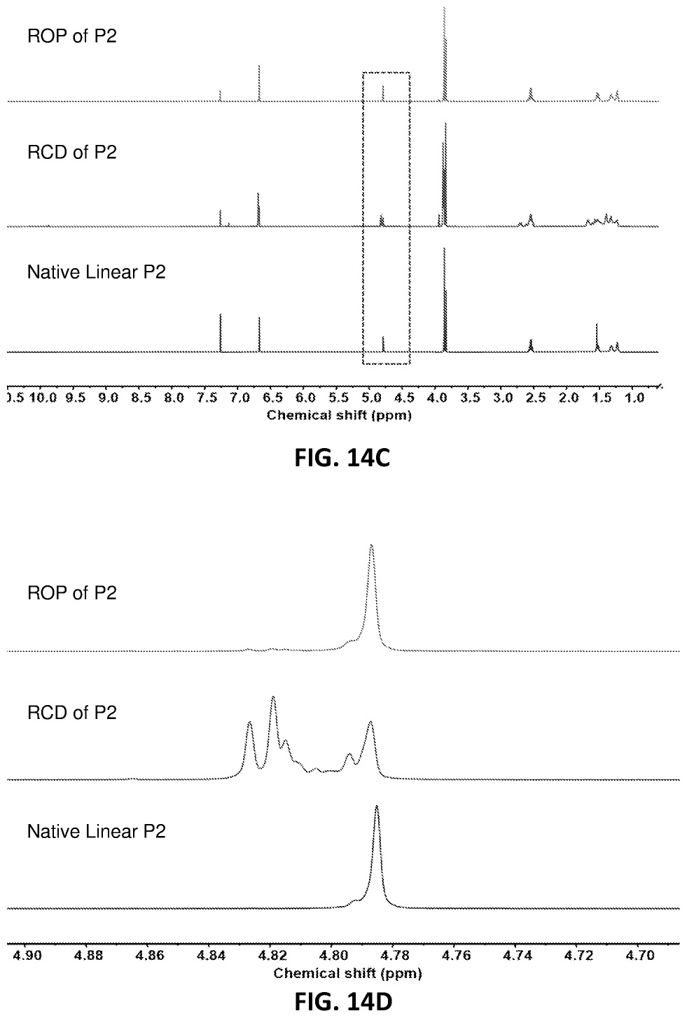

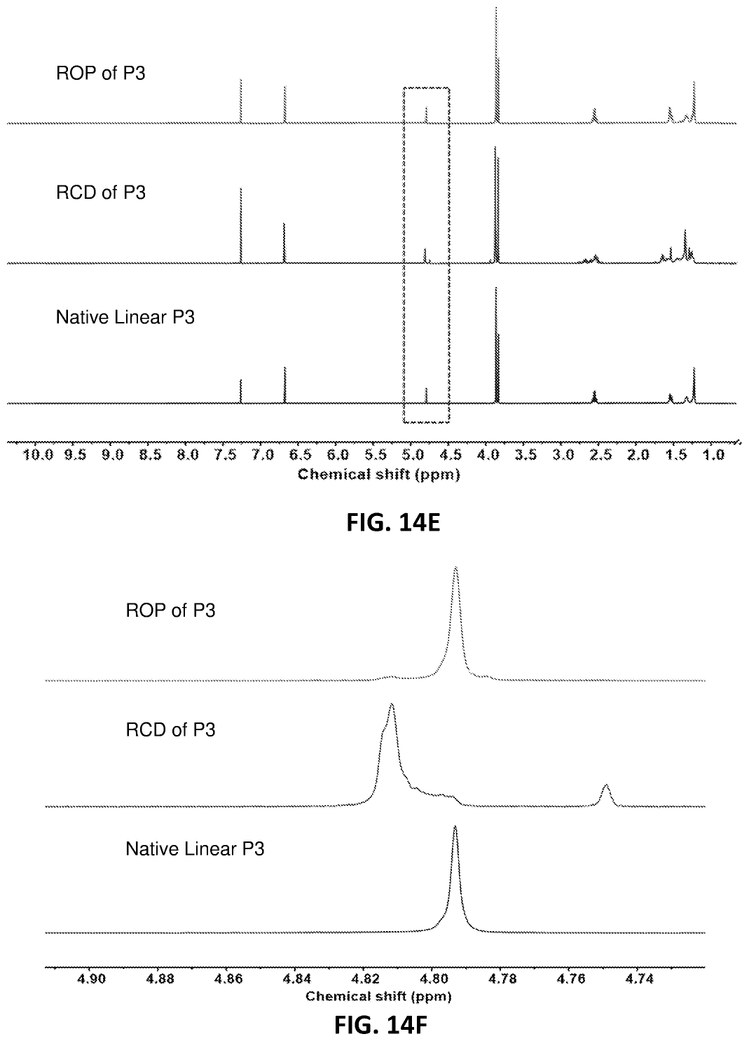

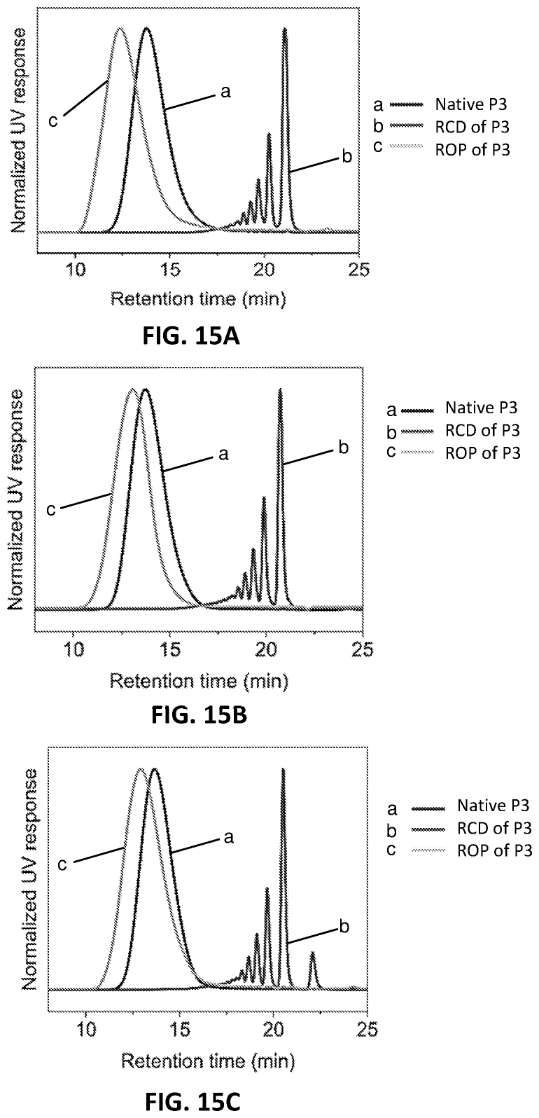

All polymers underwent RCD to give mixtures of predominantly different ring-sized dithioacetal macrocycles, suggesting an equilibrium between the cycles. The macrocyclic mixtures were obtained as white sticky powders (FIG. 13). Depolymerization resulted in new sets of peaks appearing in ¹H NMR, more deshielded than those of the native linear polymers (FIGS. 14A-14F), as well as at higher retention times in GPC (FIGS. 15A-15C). As determined by integrations of ¹H NMR peaks, the percentages of macrocycles in the mixture generated by RCD of P1, P2, and P3 were nearly 95%, 85%, and 75%, respectively. According to GPC, no residual native linear polymers were detected for any of the three systems after depolymerization (FIGS. 15A-15C). Instead, at least four different ring-sized macrocycles corresponding to m=0-3 in FIG. 12 were observed. The depolymerization pathway that occurs under high dilutions is likely the reverse of the ring-chain equilibria (Scheme 1B) initiated by random cleavage of the carbon-sulfur bond by the acid.

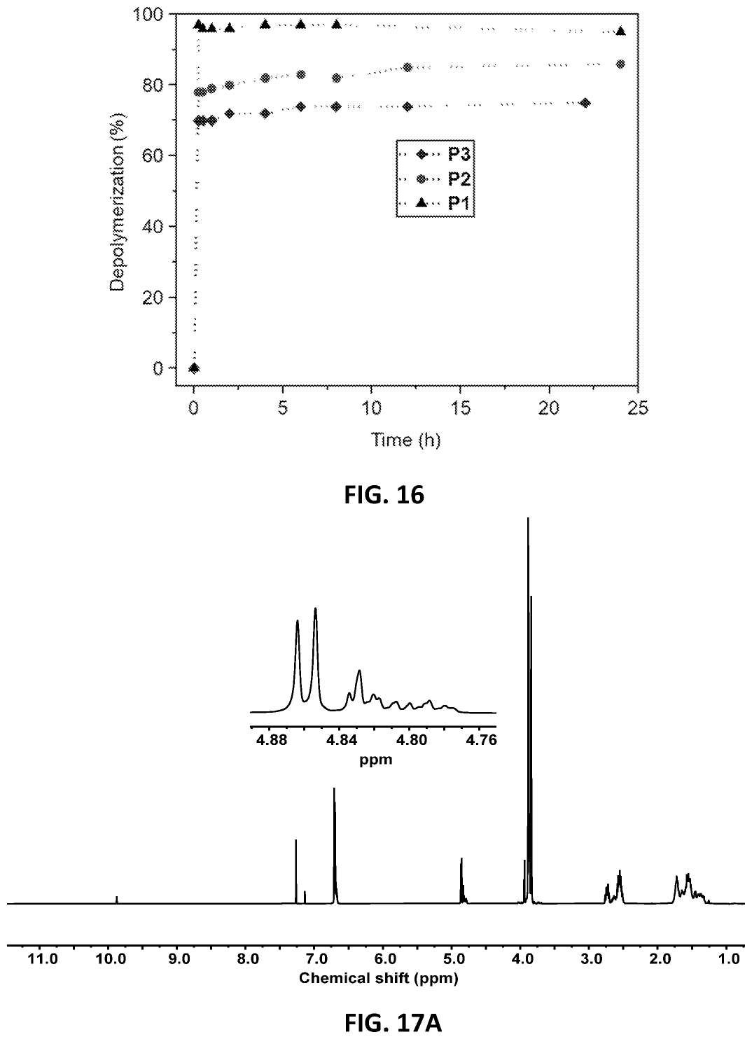

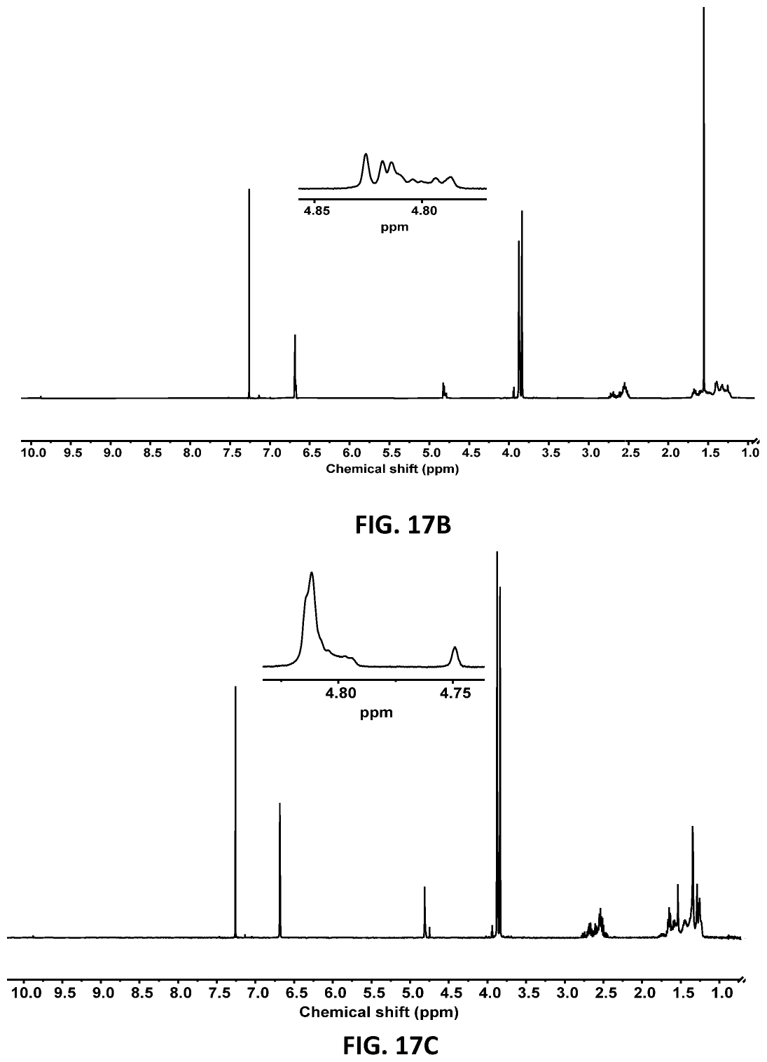

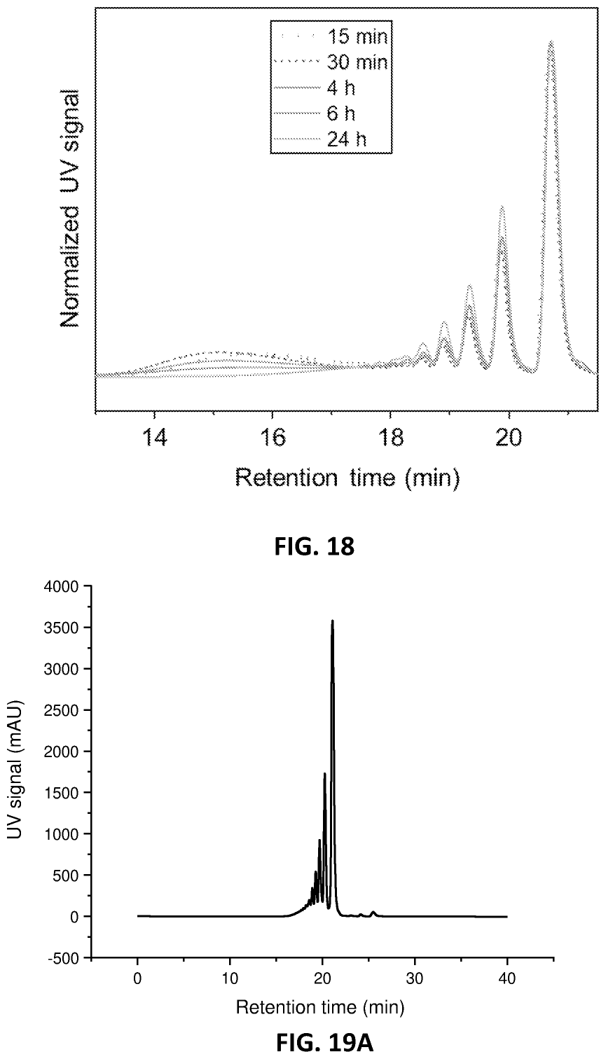

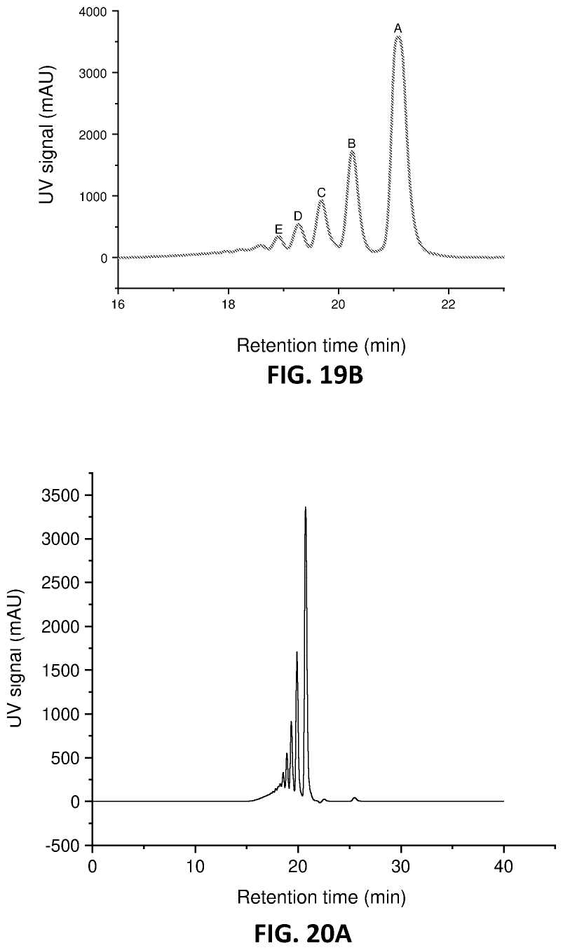

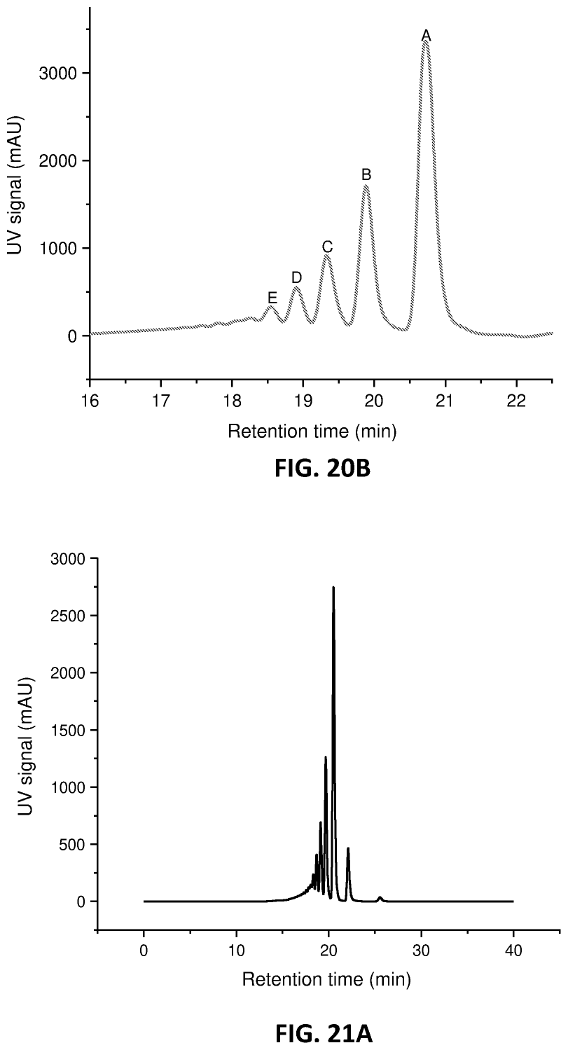

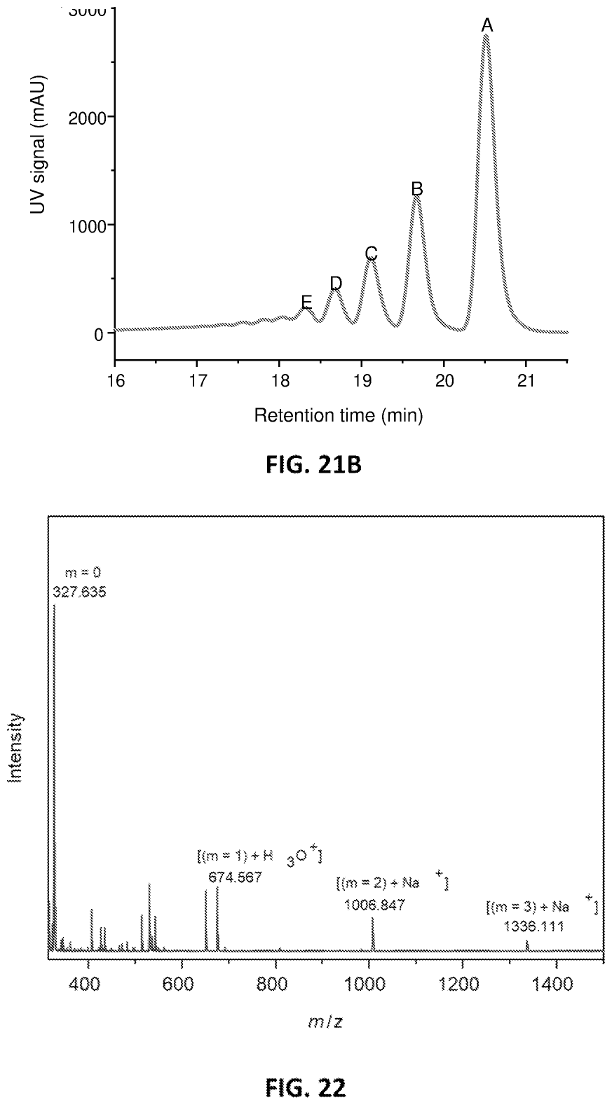

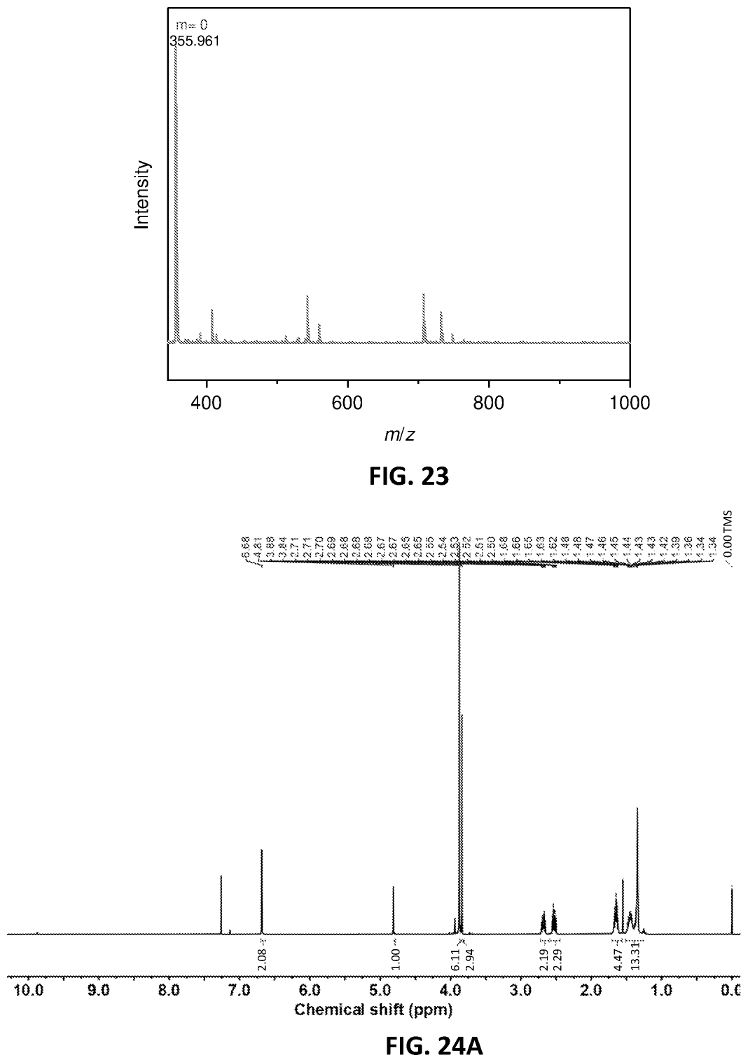

The figures show a summary of the results obtained from monitoring the RCD using ¹H NMR (see FIG. 16, with spectra shown in FIGS. 17A-17C for P1, P2, and P3, respectively), gel permeation chromatography (GPC) (e.g., see FIG. 18 with FIGS. 19A and 19B for P1, FIGS. 20A and 20B for P2, and FIGS. 21A and 21B for P3), and MALDI-TOF mass spectrometry (see FIGS. 22 and 23, for P1 and P2, respectively). Additional information concerning the GPC analyses is provided in Tables 3, 4, and 5 for P1, P2, and P3, respectively.

TABLE 3

| Peak label in | RCD | Calcd | M_{n, GPC} | |
|---|---|---|---|---|
| FIG. 19B | product | (m/z) | (g/mol) | Ð |
| A | m = 0 | 328 | 197 | 1.01 |
| B | m = 1 | 656 | 392 | 1.01 |
| C | m = 2 | 984 | 614 | 1.01 |
| D | m = 3 | 1312 | 874 | 1.00 |
| E | m = 4 | 1640 | 1180 | 1.00 |

TABLE 4

| Peak label in | RCD | Calcd | M_{n, GPC} | |
|---|---|---|---|---|
| FIG. 20B | product | (m/z) | (g/mol) | Ð |
| A | m = 0 | 356 | 262 | 1.01 |
| B | m = 1 | 712 | 519 | 1.01 |
| C | m = 2 | 1068 | 812 | 1.01 |
| D | m = 3 | 1425 | 1167 | 1.01 |

TABLE 4

| Peak label in | RCD | Calcd | M_{n, GPC} | |
|---|---|---|---|---|
| FIG. 20B | product | (m/z) | (g/mol) | Ð |
| E | m = 4 | 1780 | 1597 | 1.00 |

TABLE 5

| Peak label in | RCD | Calcd | M_{n, GPC} | |
|---|---|---|---|---|
| FIG. 21B | product | (m/z) | (g/mol) | Ð |
| A | m = 0 | 384 | 315 | 1.01 |
| B | m = 1 | 768 | 622 | 1.01 |
| C | m = 2 | 1152 | 982 | 1.01 |
| D | m = 3 | 1537 | 1411 | 1.01 |
| E | m = 4 | 1921 | 1914 | 1.00 |

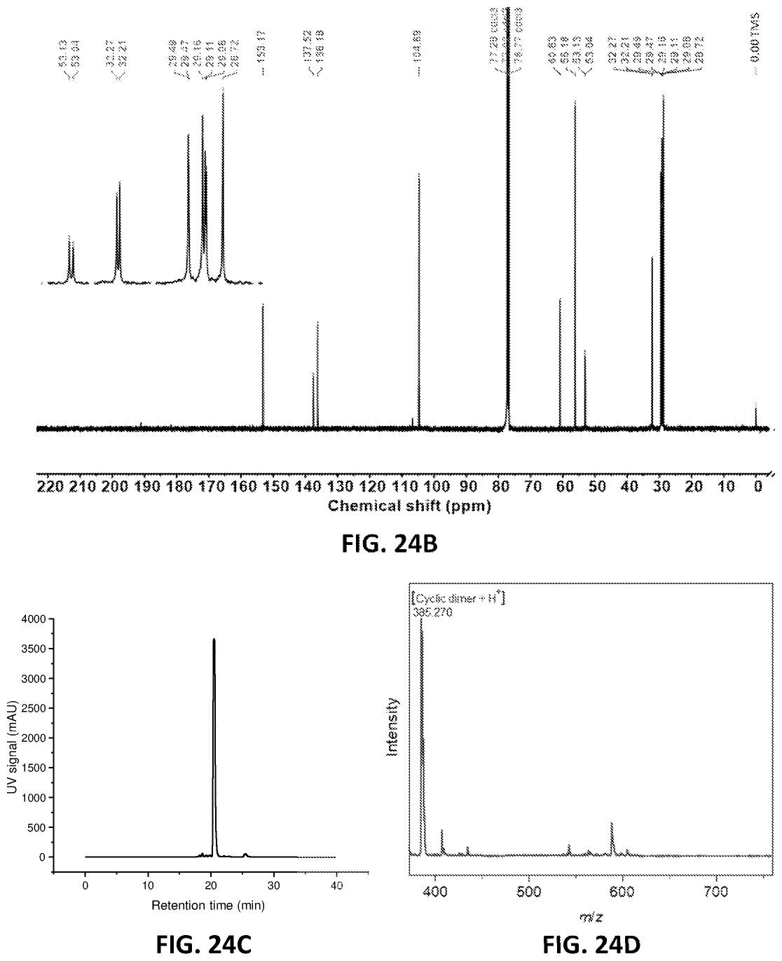

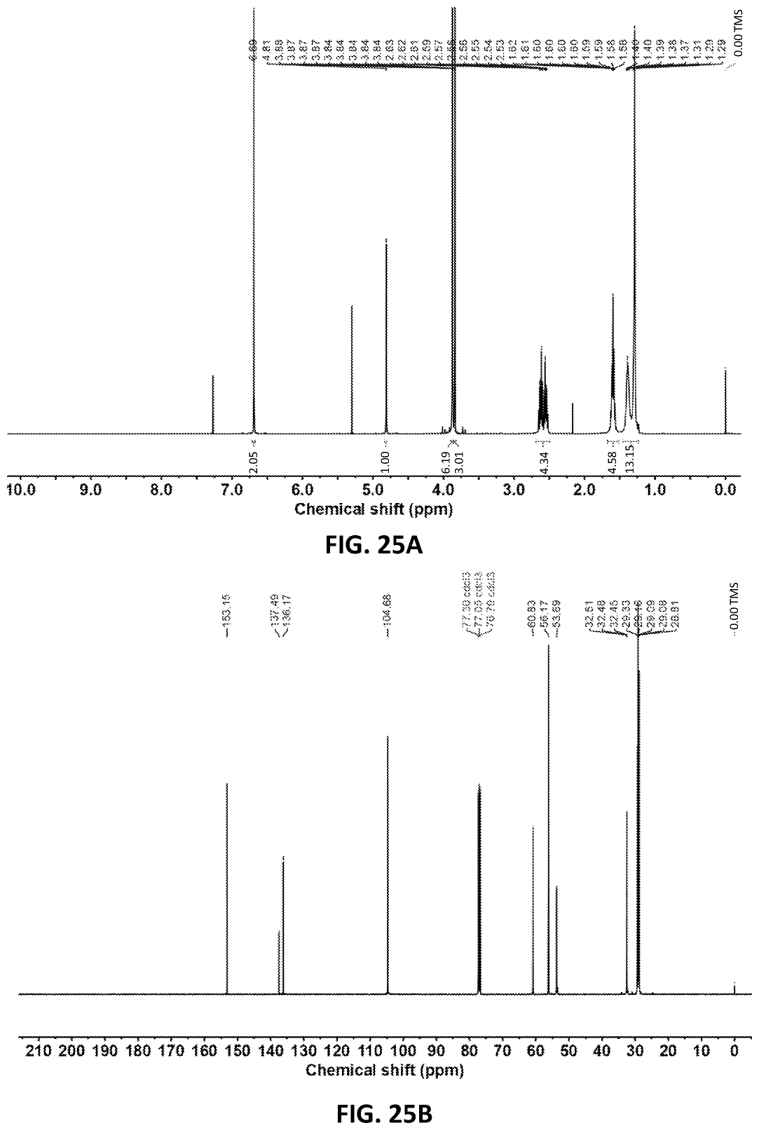

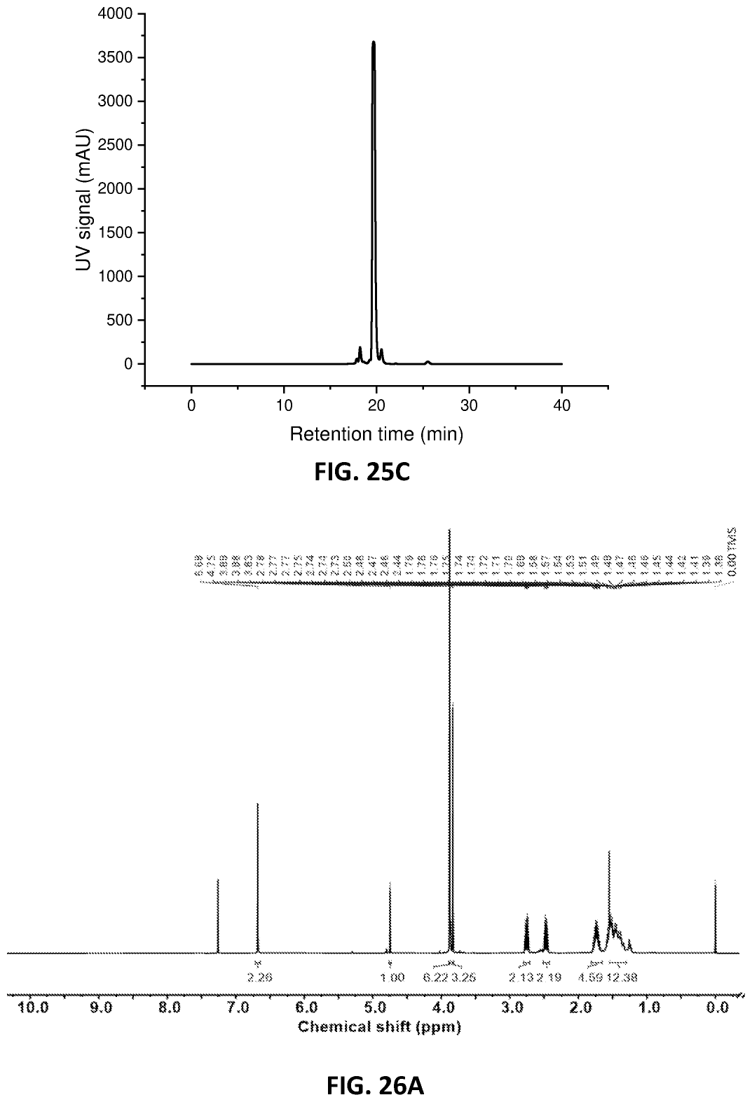

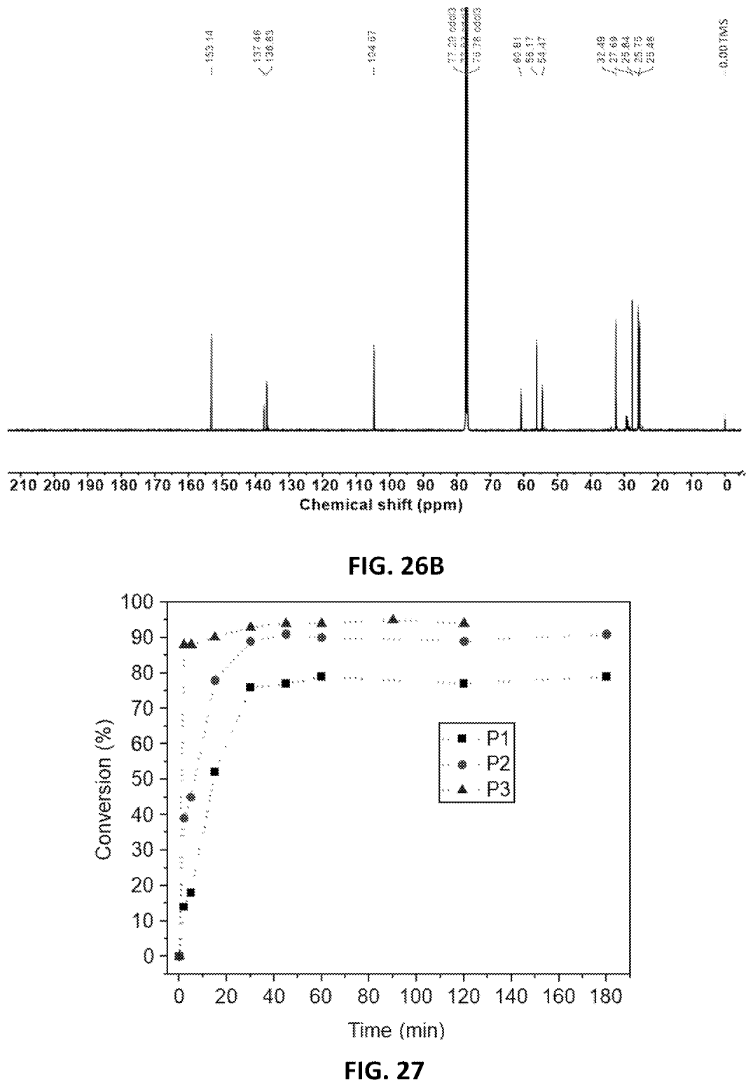

In one example, RCD of P3 was carried out at room temperature but no depolymerization occurred and only 65% depolymerization was reached at 50° C. This lack of depolymerization at lower temperatures may indicate that the ΔS for depolymerization is positive at the concentration used. In one example, when RCD of P1 was carried out at a higher concentration of 10 wt. % in refluxing toluene, depolymerization proceeded to only about 50% compared to 95% at 1.5 wt. % under otherwise same conditions, which is a characteristic of the entropy-controlled ring-chain equilibrium. In some examples, PTSA worked well for the RCD process, but resulted in brownish black reaction mixtures. Zn(II) triflate can be used to avoid the coloration. The 13-membered cyclic dimer (m=0 in FIG. 12) and 26-membered cyclic tetramer (m=1 in FIG. 12) are the major RCD products of P3 verified by ¹H (see FIG. 24A for cyclic dimer, FIG. 25A for cyclic tetramer, and FIG. 26A for another cyclic compound) and ¹³C NMR (see FIG. 24B for cyclic dimer, FIG. 25B for cyclic tetramer, and FIG. 26B for another cyclic compound), mass spectrometry (e.g., see FIG. 24D for P1), and GPC (e.g., see FIG. 24D for P1 and FIG. 25C for P2) of the isolated compounds from the RCD mixture.

As indicated above, cyclic dimer and tetramer compounds were isolated for P3, along with a macrocyclic structure, with certain characterization data provided below.

Cyclic dimer: ¹H NMR (500 MHz, CDCl₃) δ 6.68 (s, 2H), 4.81 (s, 1H), 3.88 (s, 6H), 3.84 (s, 3H), 2.74-2.62 (m, 2H), 2.59-2.46 (m, 2H), 1.72-1.58 (m, 4H), 1.53-1.28 (m, 12H). ¹³C NMR (126 MHz, CDCl₃) δ 153.17, 137.52, 136.18, 104.69, 60.83, 56.18, 53.13, 53.04, 32.27, 32.21, 29.49, 29.47, 29.17, 29.11, 29.08, 28.72; Mₙ=317 g/mol, Ð=1.01.

Cyclic tetramer: Mₙ=620 g/mol, Ð=1.01.

Other Macrocycle: ¹H NMR (500 MHz, CDCl₃) δ 6.68 (s, 2H), 4.75 (s, 1H), 3.88 (s, 6H), 3.83 (s, 3H), 2.80-2.70 (m, 2H), 2.51-2.41 (m, 2H), 1.81-1.65 (m, 4H), 1.54-1.30 (m, 12H). ¹³C NMR (126 MHz, CDCl₃) δ 153.14, 137.46, 136.63, 104.67, 60.81, 56.17, 54.47, 32.49, 27.69, 25.84, 25.75, 25.46.

### Example 3

In this example, the macrocyclic mixtures from RCD comprising multiple ring sizes were subjected to ROP (e.g., ED-ROP) in CDCl₃ using PTSA as the catalyst at room temperature (20-22° C.). Since ROP showed comparable efficiencies with both PTSA and zinc(II) triflate, the majority of the ROP runs for this example used PTSA. Concentrations of macrocycles were 15-21 wt. % based on their solubility limits and of the PTSA catalyst were 0.7-1.1 wt. %.

### Ring-Opening Polymerization (ROP)

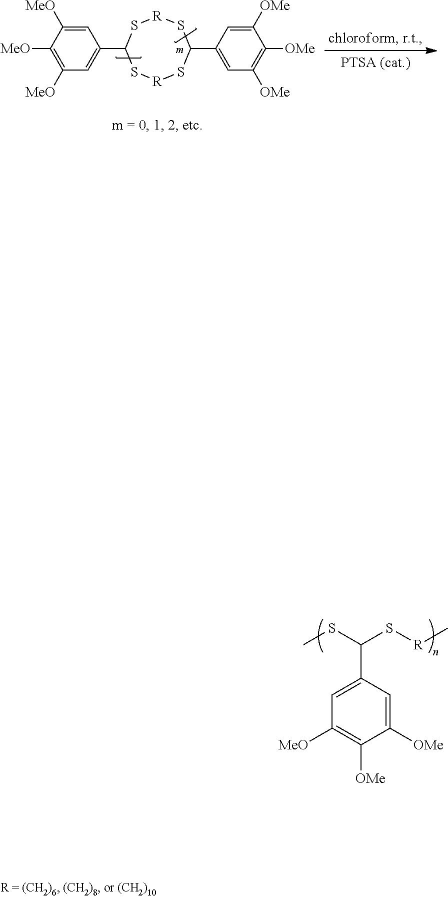

Representative experimental procedure for ROP—In a scintillation vial, the P1-RCD macrocyclic mixture (525 mg, 15 wt. %) was dissolved in CDCl₃ (2000 μL). The reaction mixture was degassed with N₂, PTSA (25 mg, 0.7 wt. %) was added, N₂ was purged again briefly, and stirred at rt. The progress of the reaction was monitored by ¹H NMR spectroscopy after quenching with triethylamine. Once the polymerization had reached equilibrium, the entire reaction was quenched with triethylamine, diluted with CHCl₃, and partitioned with water. The aqueous layer was extracted with CHCl₃ (3×). The combined organic layers were washed with water and brine, dried over Na₂SO₄, filtered, and concentrated. The crude polymer was then purified by dissolving in CHCl₃ and precipitating with diethyl ether and dried in vacuum.

The same procedure was carried out for the ROP (e.g., ED-ROP) of P2- and P3-RCD macrocyclic mixtures but with different quantities. For example, the P2-RCD macrocyclic mixture (174 mg, 18 wt. %) in CDCl₃ (540 μL) with PTSA (9 mg, 0.9 wt. %) and the P3-RCD macrocyclic mixture (895 mg, 21 wt. %) in CDCl₃ (2200 μL) with PTSA (45 mg, 1.1 wt. %) were used for the ROP process to generate P2 and P3, respectively. The yields (by weight) for ROPs were 70-80%

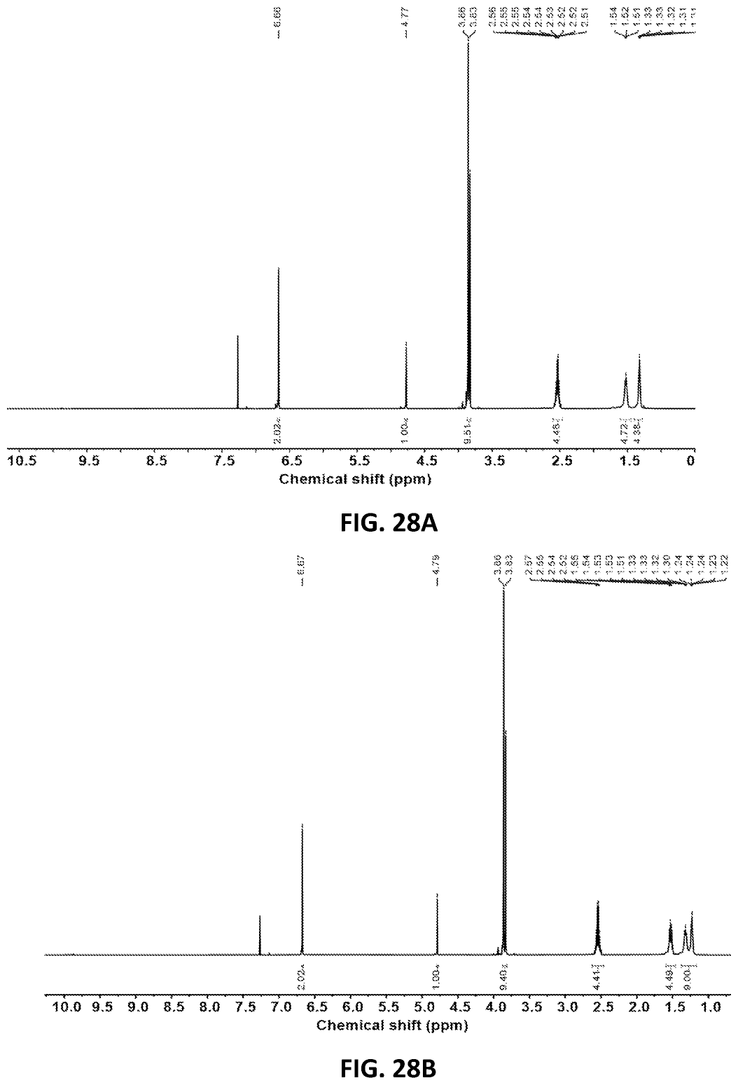

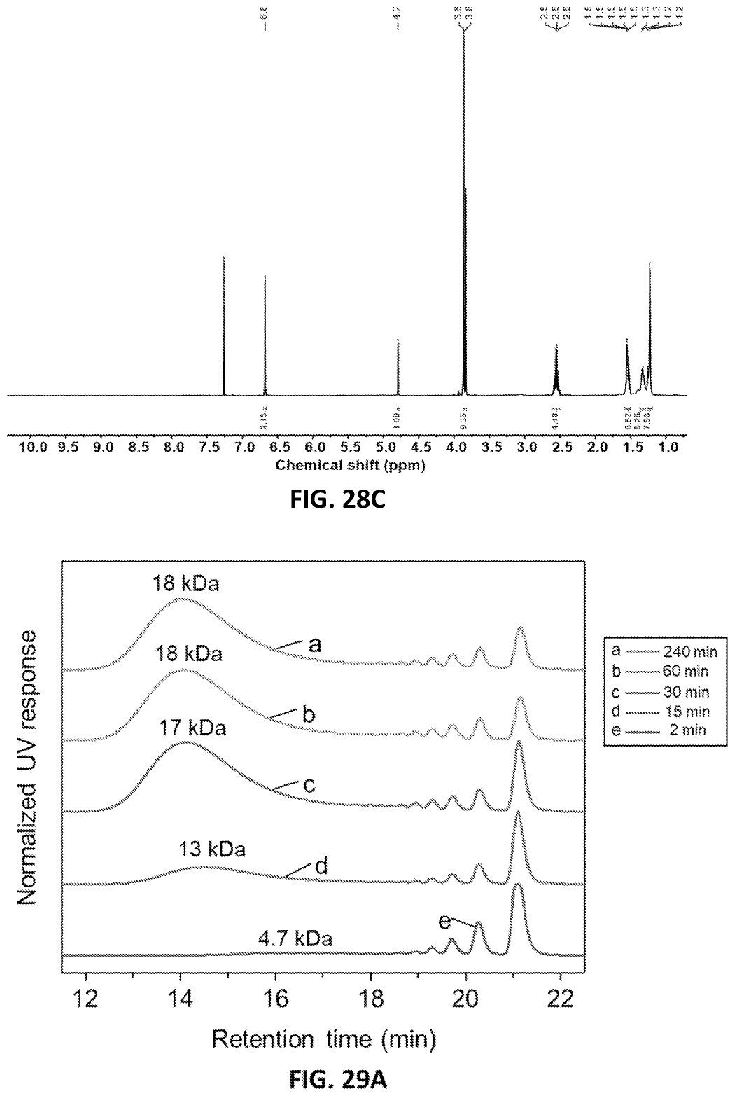

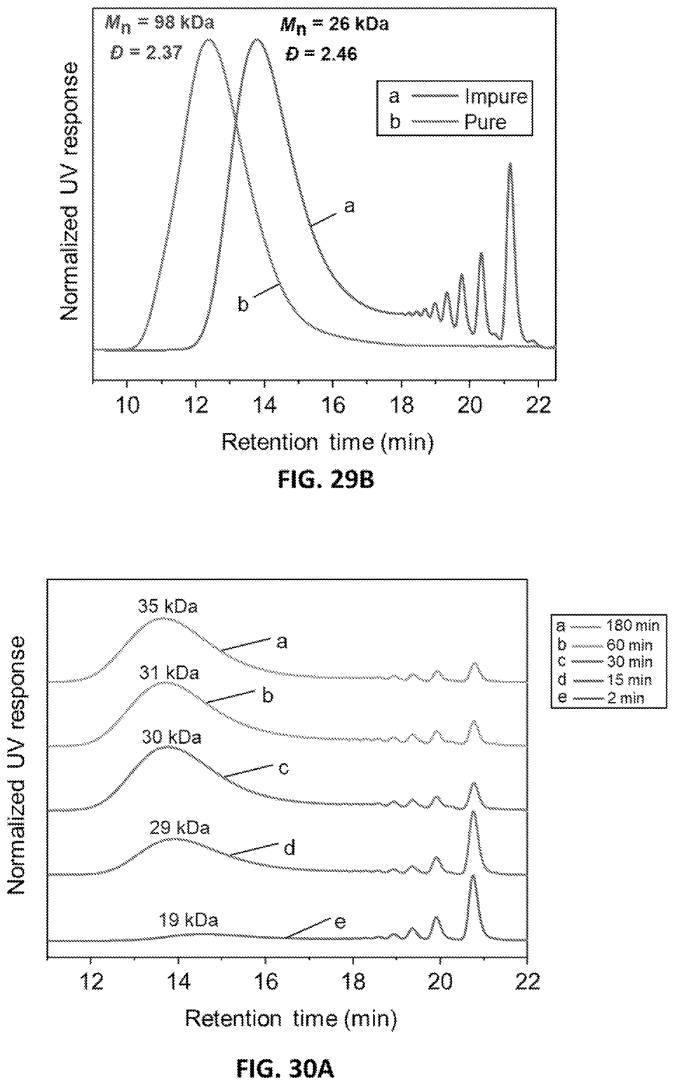

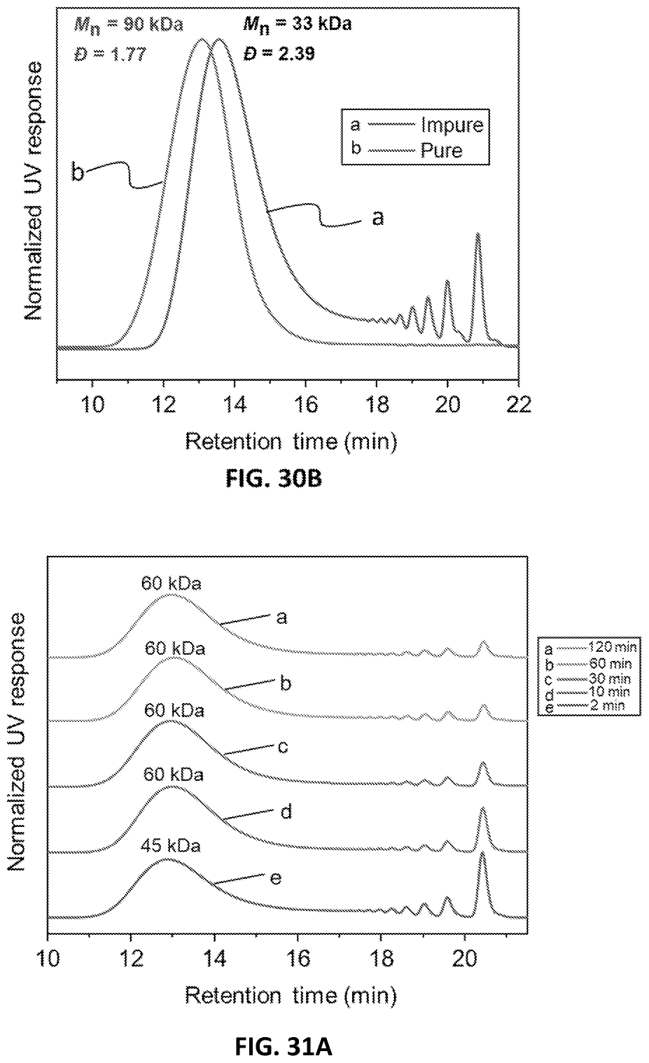

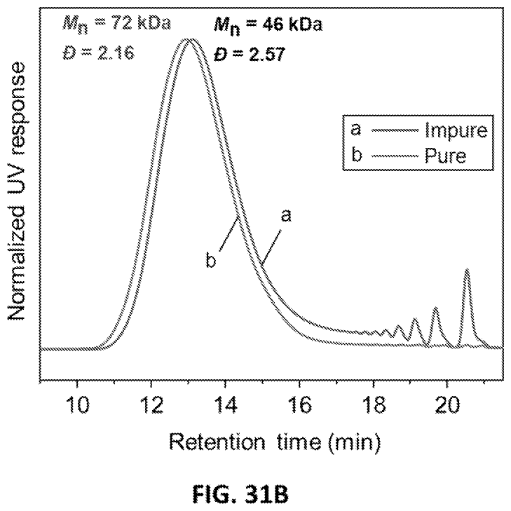

ROP for each of the three systems was monitored over time via ¹H NMR and GPC, and equilibria were reached within 2 hours for all three systems. The figures provide a summary of the results obtained from monitoring the ROP using ¹H NMR (see FIG. 27 with spectra shown in FIGS. 28A-28C for P1, P2, and P3, respectively), gas-phase chromatography (GPC) (see FIGS. 29A and 29B for P1, FIGS. 30A and 30B for P2, and FIGS. 31A and 31B for P3), and MALDI-TOF mass spectrometry. According to ¹H NMR, the conversions of macrocycles to corresponding polymers were 80%, 90%, and 95% for P1, P2, and P3, respectively. After ROP, the Mₙ and Ð had increased for all three systems compared to those of the native linear polymers (Table 6). For example, Mₙ of the polymer P1 increased from 38 to 98 kDa after recycling. The increase in molecular weight can be attributed to the intrinsic nature of the ROP process: the macrocycles render a perfect stoichiometric balance.

TABLE 6

| | |
|---|---|
| Native | After ROP |

| | M_{n, GPC} | | M_{n, GPC} | |
|---|---|---|---|---|
| Polymer | (kDa) | Ð_{GPC} | (kDa) | Ð_{GPC} |
| P1 | 38 | 1.92 | 98 | 2.37 |
| P2 | 47 | 1.66 | 90 | 1.77 |
| P3 | 53 | 1.56 | 72 | 2.16 |

### Example 4

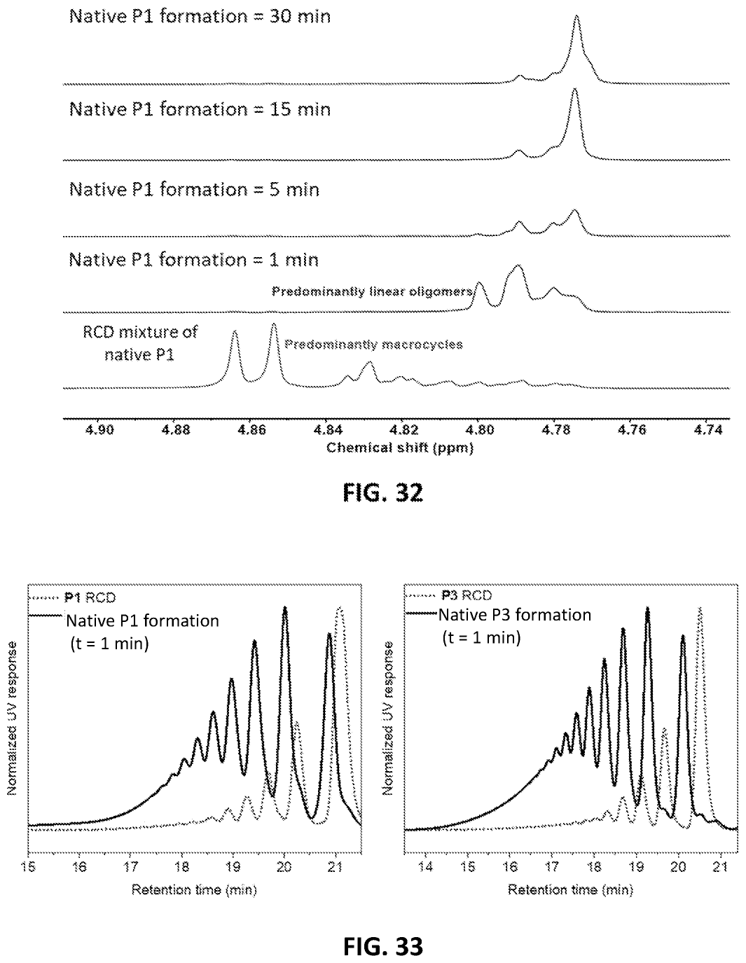

In this example, the ring-chain recycling of P1, P2, and P3 was monitored using proton NMR and GPC analysis. Results are shown in FIGS. 14A-14F and FIGS. 15A-15C. FIGS. 5A-5C, 6A-6C, FIG. 32, and FIG. 33 how data confirming that the native linear polymer formation takes place via a step-growth pathway with the initial formation of linear oligomeric species and the RCD (and ED-ROP) takes place through cyclic monomer-polymer (ring-chain) equilibria as described herein.

### Example 5

In this example, end-group analysis was conducted. Thiol end-capped oligomers were observed at low conversions and a rapid increase of molecular weight occurred at high conversions, confirming the step-growth mechanism. End-group analysis according to this example showed that the resulting polymers are linear with thiol terminal groups.

Synthesis of the control compound phenyl-thiocarbamic acid S-pentyl ester: 1-Pentanethiol (38 mg, 0.36 mmol) was dissolved in dry CDCl₃ (2.000 mL) into which dry Et₃N (85 μL, 0.61 mmol) was added along with molecular sieves (3 Å) and stirred at room temperature for ˜30 min, then PhNCO (40 μL, 0.36 mmol) was added and stirred at room temperature for 19 hours. The reaction mixture was concentrated to obtain the resulting crude phenyl-thiocarbamic acid S-pentyl ester as a white solid that was then dried in a vacuum oven. The product served as a small molecule control for ¹H NMR analysis.

Synthesis of low-molecular-weight polymers: An oven-dried scintillation vial equipped with a magnetic stir bar was charged with 1,6-hexanedithiol (376 mg, 2.50 mmol), 3,4,5-trimethoxybenzaldehyde (496 mg, 10.0 mmol), and acetonitrile (100 μL) such that the concentration of each of the two monomers were 25 M. The reaction mixture was placed in an oil bath preheated to 50° C. and let all the contents fully dissolved while equilibrating at 50° C. Activated molecular sieves (3 Å) were added and the reaction mixture was degassed. PTSA (7 mg, 0.04 mmol) was added, the mixture was purged with N₂ briefly, the vial was capped, let the reaction mixture stirred for about 5-10 min at 50° C. The reaction was quenched with triethylamine (200 μL), dissolved in CH₂Cl₂, and partitioned with distilled water. The CH₂Cl₂ layer was separated, and the aqueous layer was extracted two more times with additional CH₂Cl₂, the combined organic layers were washed with water and brine, dried over anhydrous Na₂SO₄, filtered, and concentrated. The polymer was then purified by dissolving in CH₂Cl₂ and precipitating with diethyl ether (×3). The polymer was dried in vacuum. M_{n, GPC}=5,604 g/mol, Ð=1.74 and M_{n, GPC}=10,732 g/mol, Ð=2.04 for two batches of polymer products.

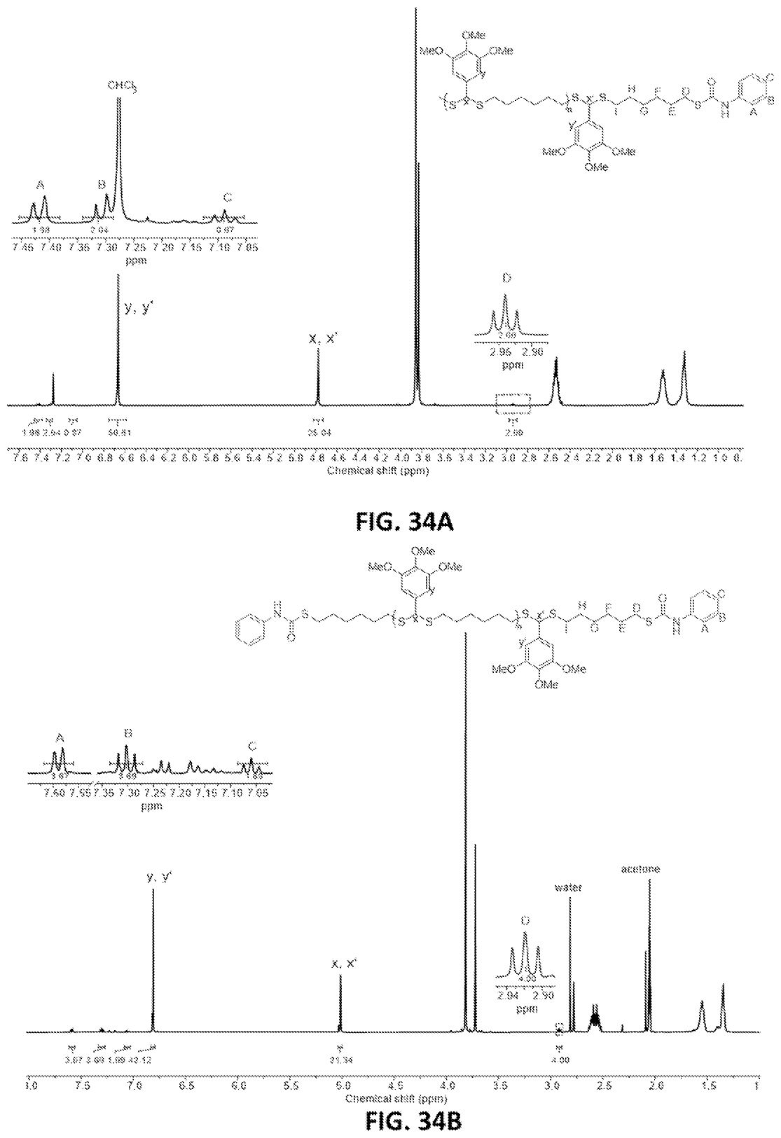

Synthesis of phenyl-thiocarbamate tethered poly(dithioacetal): In a scintillation vial, the above polymer (265 mg, Mₙ₋ 10,732 g/mol, 0.025 mmol) was dissolved in dry CDCl₃ (1.000 mL) and was added dry Et₃N (6 μL, 0.043 mmol) along with molecular sieves (3 Å). Let the mixture stir at room temperature for 30 min. Nitrogen was purged briefly, PhNCO (2.8 μL, 0.026 mmol) added, N₂ was purged again, the vial was capped, and the reaction mixture was stirred at room temperature for 22 hours. The reaction mixture was diluted with fresh CHCl₃ and purified by precipitating with diethyl ether (×3). The polymer was dried in vacuo. FIG. 34A shows the ¹H NMR spectrum (400 MHz, CDCl₃) for the product.

Synthesis of bisphenyl-thiocarbamate tethered poly(dithioacetal): In a scintillation vial, the above polymer (390 mg, Mₙ=5,604 g/mol, 0.0695 mmol) was dissolved in dry CDCl₃ (2.000 mL) and was added dry Et₃N (68 μL, 0.486 mmol) along with molecular sieves (3 Å). Let the mixture stir at room temperature for 30 min. Nitrogen was purged briefly, PhNCO (30 μL, 0.278 mmol) added, N₂ was purged again, the vial was capped, and the reaction mixture was stirred at room temperature for 21 hours. Note the reaction mixture had turned from clear/colorless to turbid. Reaction was diluted with acetone and purified by precipitating with diethyl ether (×3). The polymer was dried in vacuum. FIG. 34B shows the ¹H NMR spectrum (500 MHz, acetone-d₆) for the product.

### Example 6

Increase in molecular weight of unpurified polymers over time—All three polymers (P1, P2, and P3) were synthesized following the representative experimental procedure but no purification was carried out. Consequently, the PTSA catalyst remained in the reaction mixture. The reaction mixtures were monitored by GPC over time and the molecular weight data are shown below in FIGS. 9-11 and Tables 2A-2C.

### Example 7

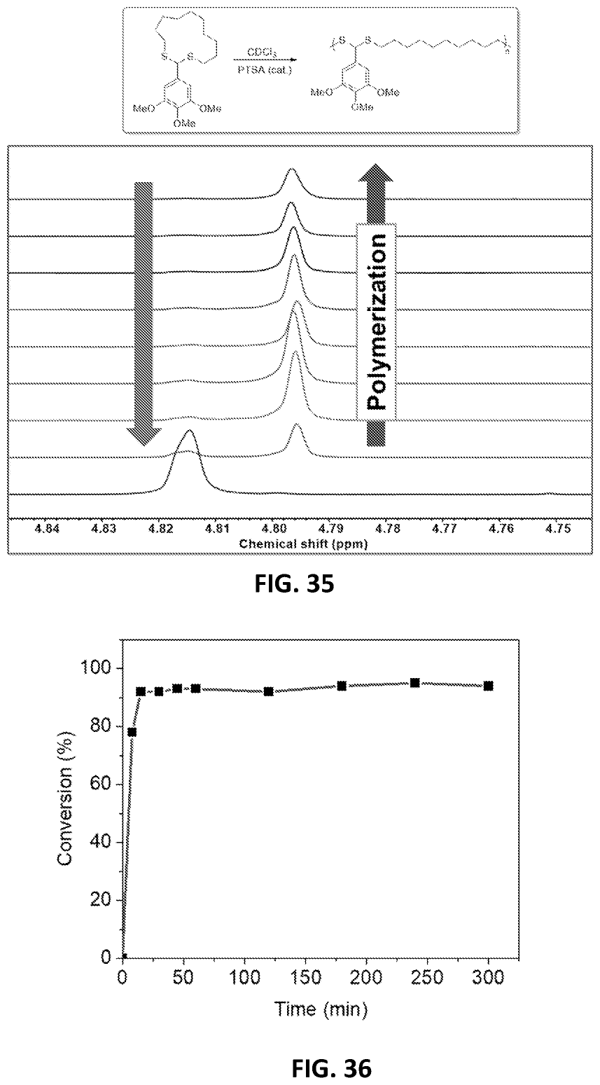

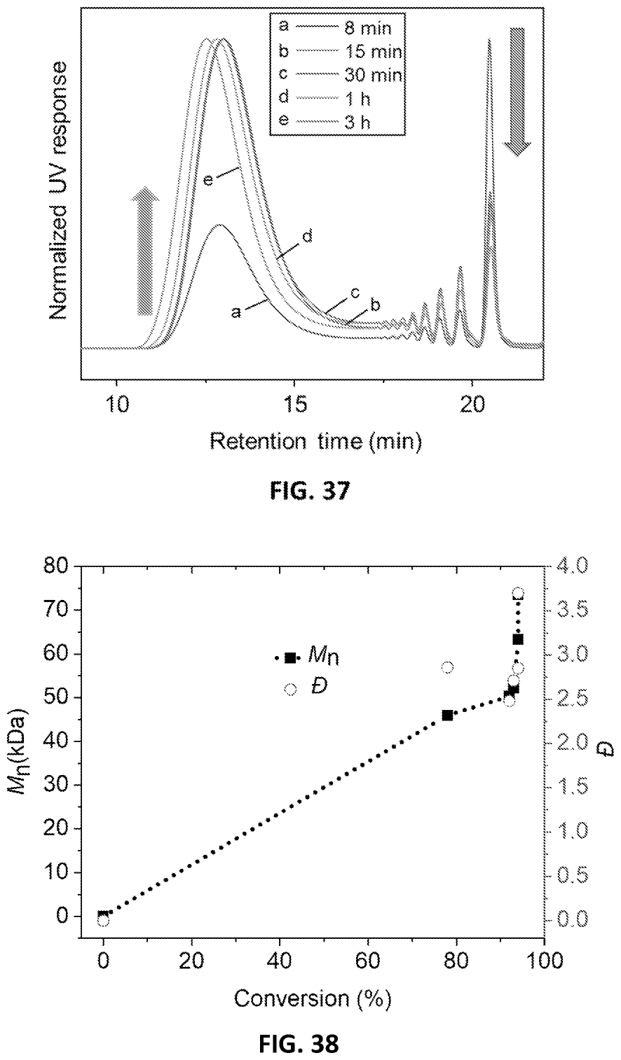

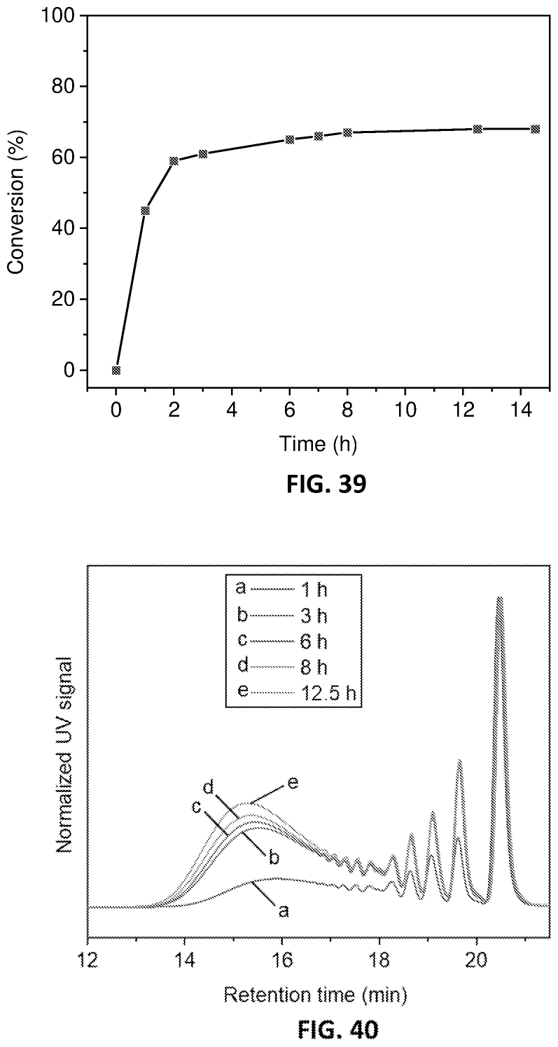

In this example, polymerization kinetics were evaluated using the 13-membered cyclic dithioacetal dimer isolated from the depolymerization product of P3. Results are shown in FIGS. 35-41. The 13-membered macrocycle (M) from RCD mixture of P3 was isolated via flash chromatography (SiO₂) with 100% CH₂Cl₂→2% acetone in CH₂Cl₂. The macrocycle M was dissolved in CDCl₃ to make two stock solutions at concentrations of 0.50 and 0.08 mol/L. Each of the stock solutions was equally distributed between four vials, which were placed in oil-baths preheated to 30, 40, and 50° C., while one was left at rt. All were allowed to equilibrate at the desired temperature for about 15 minutes. Then PTSA was added to each vial separately to have a final concentration of 0.25 mol/L (t=0). The conversions over time were determined by ¹H NMR after quenching aliquots of reaction mixtures with Et₃N in CDCl₃ for room temperature sample. Once the room temperature sample reached the equilibrium, the reactions at higher temperatures were also quenched in the same way while keeping the quenched mixture at the respective temperature. Note that by the time room temperature sample reached the equilibrium, reactions at higher temperatures should also have reached equilibrium based on our preliminary kinetic studies. Some of the quenched reaction mixtures (at rt) used for NMR analyses were concentrated and dried in vacuum overnight prior to running GPC.

Rapid polymerization was observed for [M]₀=0.50 mol/L (11 wt. % of monomer and 2.8 wt. % of PTSA). The system at room temperature reached equilibrium at 3 hours with a 94% conversion. When [M]₀ was reduced to 0.08 mol/L (2.2 wt. % of monomer and 3.0 wt. % of PTSA), it took about 12 hours for the system to reach equilibrium yet only with a 68% conversion (FIG. 42). When [M]₀=1.00 mol/L, the macrocyclic solution became supersaturated and tended to crash out during transferring of the stock solution. A relatively large amount of PTSA was employed in these studies. This was solely based on practical convenience: the systems at room temperature would have taken several days to attain equilibrium with a much lower amount of PTSA, particularly with a lower initial monomer concentration. A higher temperature favors a higher conversion of the monomer. For example, when [M]₀=0.50 mol/L, the conversion was 94% at equilibrium at 21° C., which increased to 99% at 50° C. This represents an advantage of low-Tr ED-ROP compared to the enthalpy-driven ROPs, which are typically carried out at low temperatures to compensate for the entropic penalty.

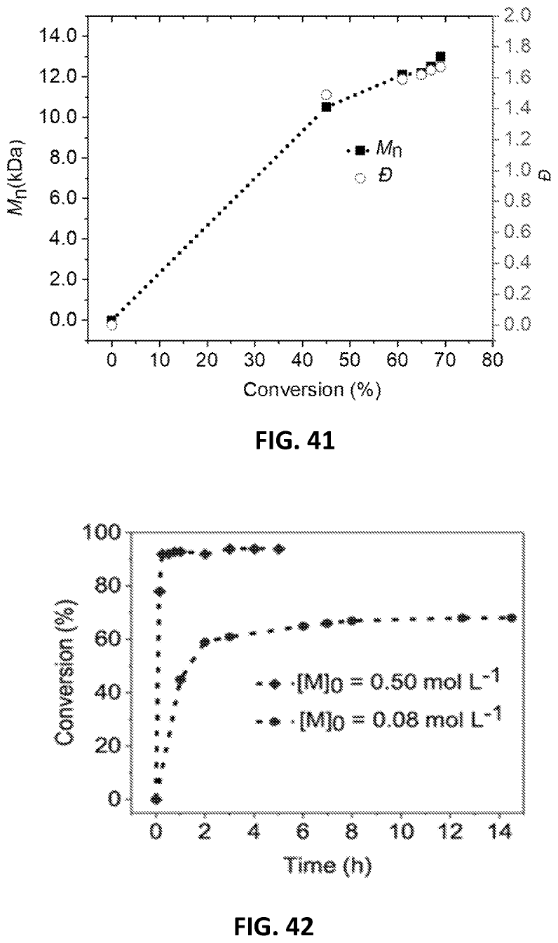

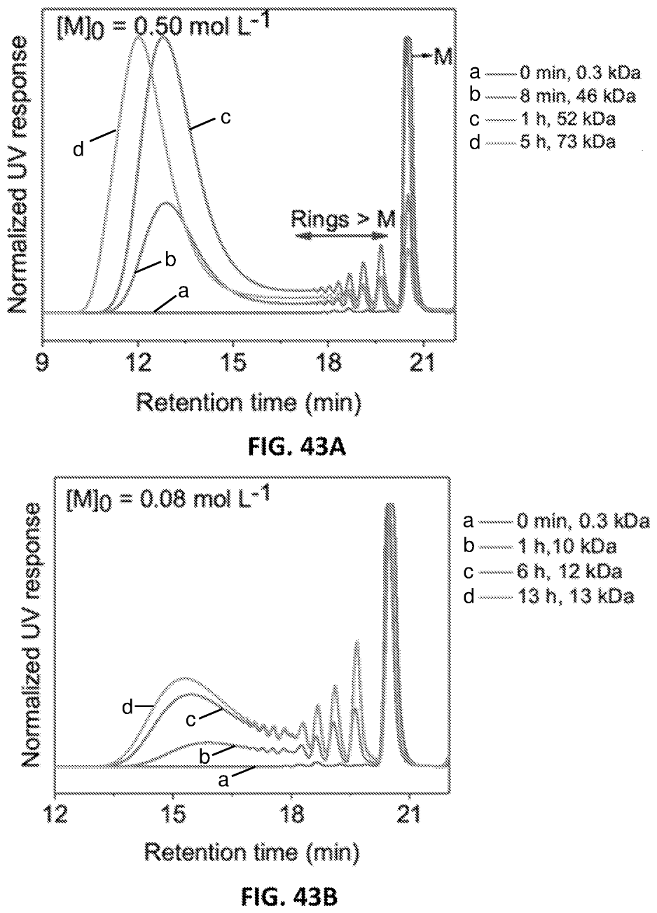

The molecular weight over conversion was measured for both concentrations. GPC showed that polymers and cyclic oligomers were formed in parallel (FIGS. 43A and 43B), indicating that the cationic propagating end is prone to back-biting. When [M]₀=0.50 mol/L, a rapid increase of molecular weight was observed at conversions above 92% along with high dispersities (FIG. 38). Polymerization at the lower monomer concentration of 0.08 mol/L exhibited more living nature and partial control of the dispersity (Ð=1.49-1.67), but produced low molecular weight products (FIG. 41). Although the mechanism shown in Scheme 1B is plausible and could be one non-limiting theory, it currently is believed that that initiation by residual benzaldehyde species present in the monomer M is also possible. Since benzaldehyde is more basic than dithioacetal, it would react with the acid to form a highly reactive carbon cation that serves as an efficient initiator for the ROP (Scheme 2, below) of the cyclic dimer.

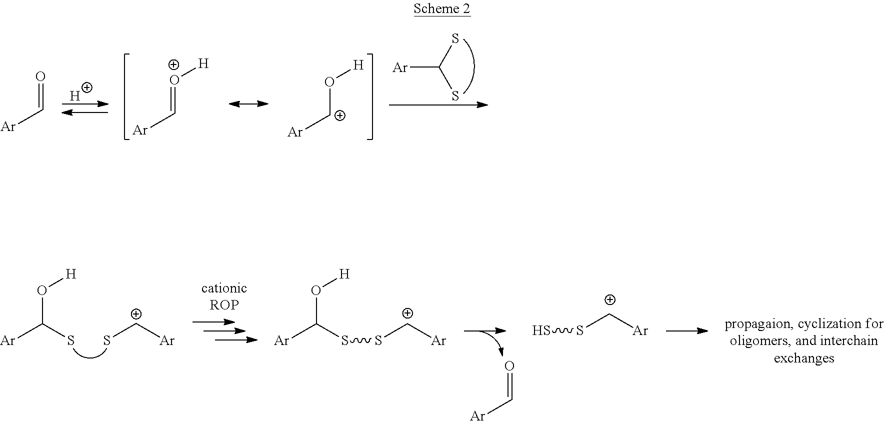

### Example 8

In this example, the thermodynamics of polymerization was evaluated. The thermodynamic parameters measured for the ROP of M are summarized in Table 7. Additional information is provided in Table 8. A positive ΔSₚ was obtained which can be qualitatively justified by a large increase in conformational freedom as the 13-membered macrocycle M converts to the corresponding polymer. Curiously, when [M]₀ was nearly six times lower, the absolute values of both ΔHₚ and ΔSₚ decreased by an order of magnitude (Table 7). If the trend holds for even lower monomer concentrations, the entropy change would further decrease, making polymerizations at quite low concentrations thermodynamically unfavorable. This concentration dependency of the monomer-polymer equilibrium is responsible for the recyclability. It was predicted that polymerization of the respective cyclic dimers resulting from RCD of P1 (9-membered ring) and P2 (11-membered ring) would have somewhat higher T_{f} values compared to that of the macrocycle derived from P3 (i.e., M: 13-membered ring) based on their lower conversions in ROP. The smaller cyclic dithioacetals have a higher loss of translational entropy upon polymerization.

TABLE 7
Thermodynamic parameters for the polymerization
of 13-membered cyclic monomer M obtained experimentally
at two monomer concentrations.

| [M]₀ | [PTSA]₀ | ΔHₚ | ΔSₚ | T_{f} | ΔGₚ at 298K |
|---|---|---|---|---|---|
| (mol/L) | (mol/L) | (kJ/mol) | (J/mol K) | (° C.) | (kJ/mol) |
| 0.50 | 0.25 | 38.8 | 160 | −22 | −8.9 |
| 0.08 | 0.25 | −4.0 | 16 | N/A | −8.9 |

TABLE 8
Thermodynamics of polymerization

| T | 1/T × 10³ | [Cyclic dimer]_{eq} | In([Cyclic |
|---|---|---|---|
| (K) | (K⁻¹) | (mol L⁻¹) | dimer])_{eq} |
| 294 | 3.40 | 29.42 | −3.5261 |
| 303 | 3.30 | 22.91 | −3.7762 |
| 313 | 3.19 | 14.77 | −4.2152 |
| 323 | 3.10 | 6.99 | −4.9633 |

The natural logarithm of [M]_{eq} was plotted as a function of the inverse of the absolute temperature (i.e., Van′t Hoff analysis), based on the following equation

$${ln [ M ]}_{eq} = Δ {H}_{p}^{o} / RT - Δ {S}_{p}^{o} / R$$

where R is the universal gas constant. From the slope and intercept ΔHₚ and ΔSₚ were deduced. Floor temperature T_{f} was calculated at [M]₀=0.50 mol/L, based on the equation below.

$${T}_{f} - Δ {H}_{p}^{o} / ( Δ {S}_{p}^{o} + R {ln [ M ]}_{0} )$$

### Example 9

In this example, polymers were synthesized using 4-anisaldehyde or 4-nitrobenzaldehyde as the aldehyde derivative. An oven-dried scintillation vial equipped with a magnetic stir bar was charged with 1,6-hexanedithiol (752 mg, 5.00 mmol) and either 4-anisaldehyde (695 mg, 5.00 mmol) or 4-nitrobenzaldehyde (771 mg, 5.00 mmol) in a solvent free environment. The reaction mixture was placed in an oil bath preheated to 50° C. and let equilibrate at 50° C. Activated molecular sieves (3 Å) were added and the reaction mixture was degassed. PTSA (10 mg, 0.05 mmol) was added, the mixture was purged with N₂ briefly, the vial was capped, let the reaction mixture stirred for 24 hours at 50° C. After 24 hours, the reaction was cooled to rt, dissolved in CHCl₃, and partitioned with saturated NaHCO₃ solution. The CHCl₃ layer was separated, and the aqueous layer was extracted two more times with additional CHCl₃, the combined organic layers were washed with water and brine, dried over anhydrous Na₂SO₄, filtered, and concentrated. The polymer was then purified by dissolving in CHCl₃ and precipitating with diethyl ether (×3). The polymer was dried in vacuum. Polymer between 1,6-hexanedithiol and 4-anisaldehyde: M_{n, GPC}=34,493 g/mol, Ð=1.57; Polymer between 1,6-hexanedithiol and 4-nitrobenzaldehyde: M_{n, GPC}=63,058 g/mol, Ð=1.97

### Example 10

In this example, the general procedure for native linear polymer synthesis as mentioned in Example 1 was followed to synthesize polymer compounds using 3,4,5-trimethoxybenzaldehyde (248 mg, 1.25 mmol), PTSA (3 mg, 0.01 mmol) in acetonitrile (50 μL) with either 1,4-butanedithiol (153 mg, 1.25 mmol) or 1,5-pentanedithiol (177 mg, 1.25 mmol) to synthesize polymers where R═(CH₂)₄ or (CH₂)₅, respectively. The amount of polymer formation was virtually negligible under both conditions in this particular example.

### Example 11

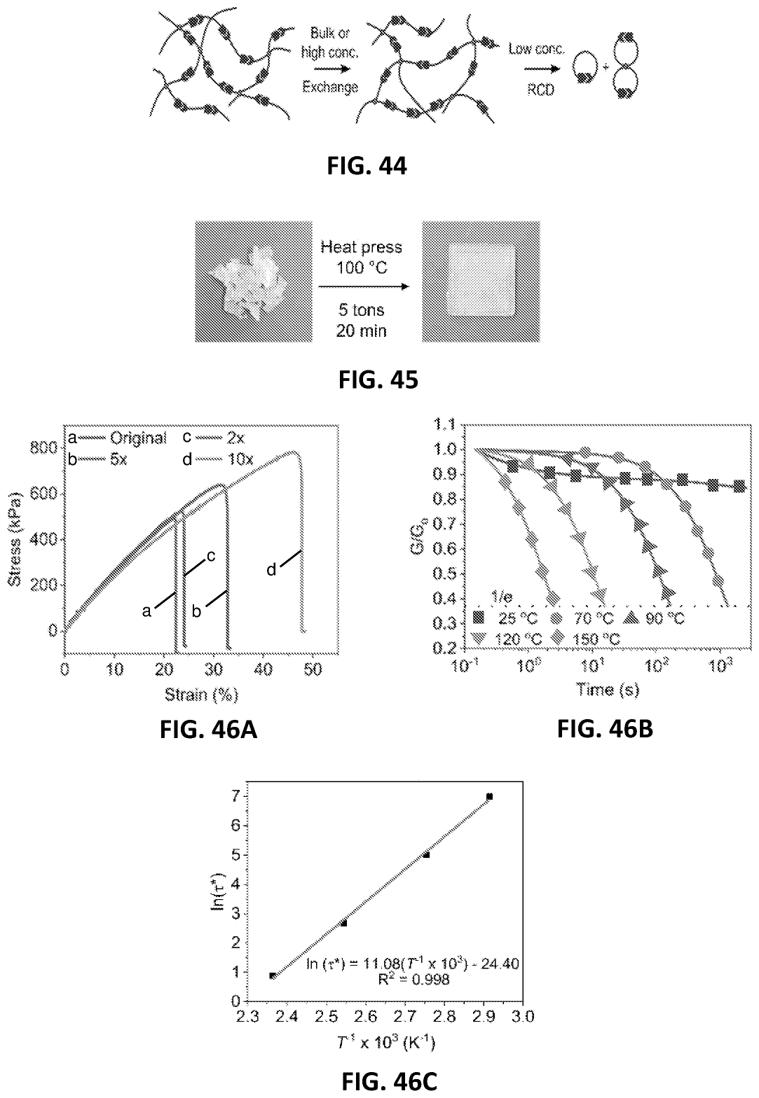

In this example, the synthesis of crosslinked P3 (containing PTSA) was evaluated (FIG. 44). Crosslinked P3 was synthesized by reacting 3,4,5-trimethoxybenzaldehyde and 1,10-decanedithiol in the presence of terephthaldehyde as the crosslinker with a 10:12:1 molar ratio. PTSA was used as the catalyst, which remained in the resulting polymer to catalyze the dynamic exchange reactions. In this specific example, an oven dried scintillation vial equipped with a magnetic stir bar was charged with the 1,10-decanedithiol (1264 mg, 6.00 mmol, 12 equiv, f=2), 3,4,5-trimethoxybenzaldehyde (991 mg, 5.00 mmol, 10 equiv, f=2), terephthaldehyde (67 mg, 0.50 mmol, 1 equiv, f=4), the vial was immersed in an oil-bath preheated to 70° C. and let equilibrate. Acetonitrile (240 μL) was added to fully dissolve the contents and activated molecular sieves (3 Å) were added. The reaction mixture was degassed, PTSA (9.5 mg, 0.05 mmol, 0.1 equiv) was added, degassed again, the vial was sealed, let the reaction mixture stir at 70° C. for 3 hours. After drying at room temperature for 24 hours, the resulting polymer was divided into smaller pieces and placed into a square mold. The sample was then processed in a heat press at 100° C. for 20 min with 5 tons of applied pressure to create the films (25 mm×25 mm×1 mm). While the P3 polymer is a soft and sticky thermoplastic without any noticeable elasticity, the crosslinked P3 is a soft elastomer like silicone rubber. It swelled in chloroform with triethylamine without dissolution. The newly formed polymer was heat pressed with 5 tons of pressure at 100° C. for 20 min to obtain a testable film (original crosslinked P3, FIG. 45).

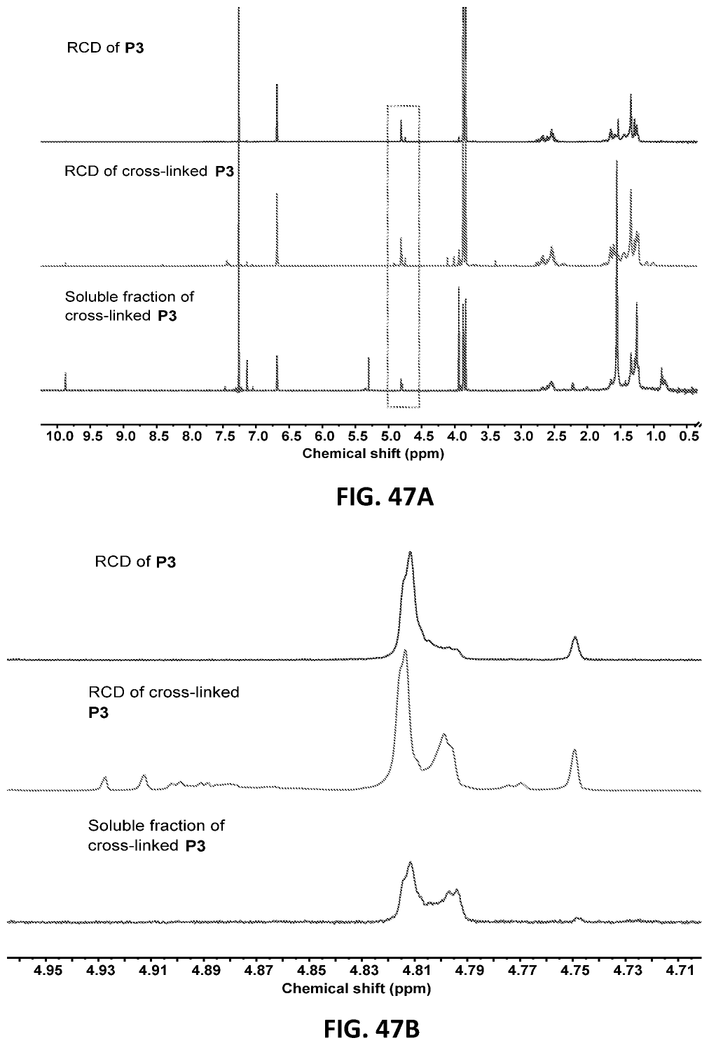

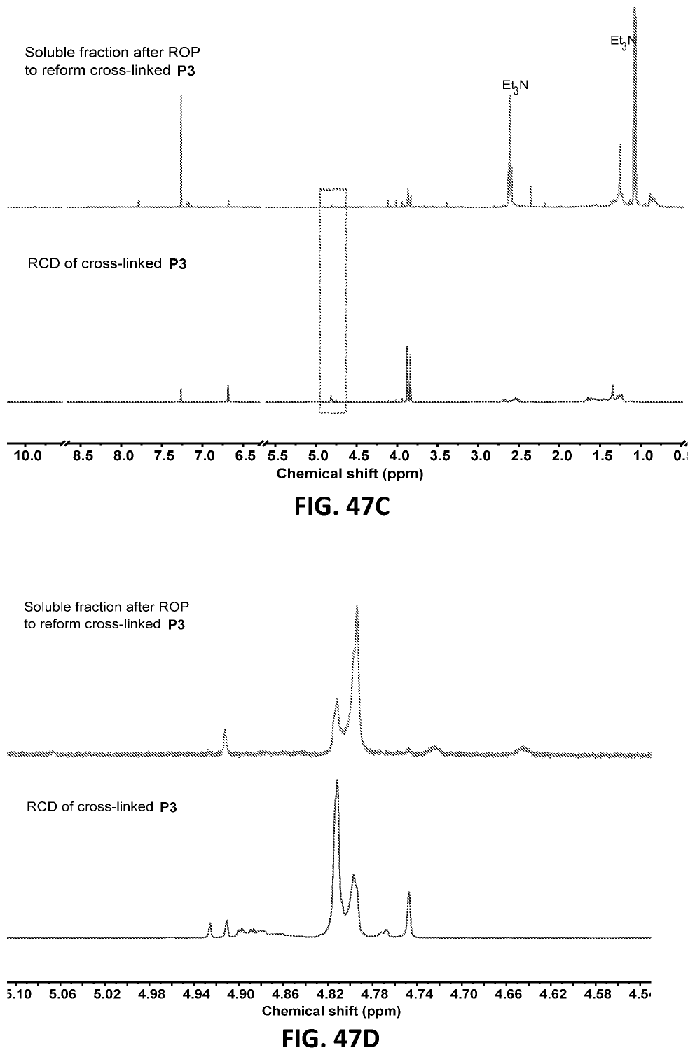

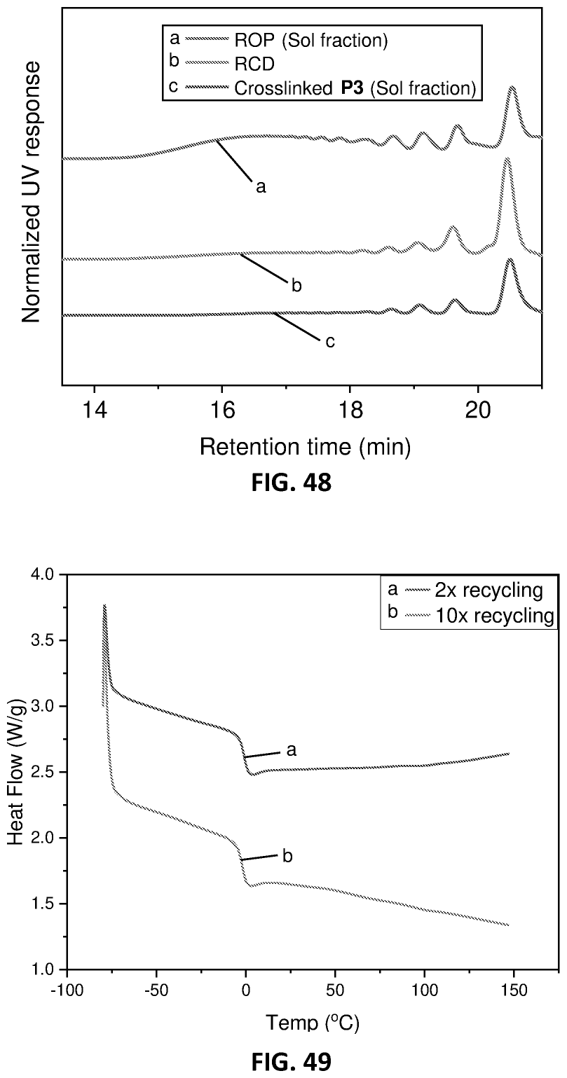

The tensile measurements were performed on the network through every round of reprocessing from the original pressed film to 10 times of recycling. The reprocessing procedure was identical to the initial film formation (5 tons applied pressure, 100° C., 20 min). As shown in the tensile results (FIG. 46A and Table 9), there is a noted increase in toughness as the number of reprocessing cycles increases. This may be caused by the polymerization of residual macrocycles into the polymers as the film underwent more cycles of thermal recycling, producing a more homogeneous film with fewer defects. The soluble fraction was measured, which consists of significant amounts of macrocycles (FIGS. 47A-47D and 48), and it decreased from ˜12 wt. % for the original polymer to ˜4.0 wt. % after 10 times of heat reprocessing. The polymer would also become softer due to increased chain lengths between crosslinks, which is in agreement with the slight decrease of the Young's moduli and the T_{g} after the 10th recycling. The T_{g} of the material decreased from −0.9° C. after 2 rounds of recycling to −2.0° C. after 10 rounds of recycling (FIG. 49).

The dynamic nature of the material was characterized through shear stress-relaxation experiments. As shown in FIG. 46B, the network relaxed the stress admirably at increased temperatures. The relaxation times decreased from 3 hours at 70° C. to ˜4 min at 150° C. (Tables 10A-10D). These results were plotted using an Arrhenius adjacent model, which gave a linear correlation within 70-150° C. temperature range (FIG. 46C). The activation energy for viscous flow (E_{A}) remained ˜32 kJ/mol for the first 5 cycles. However, after the 10th recycling, the relaxation time increased significantly, and the activation energy increased to 35 kJ/mol (Table 9 herein). Without being limited to a single theory, it currently is believed that this results from possible thermal degradation of the PTSA catalyst.

### Example 12

In this example, RCD and ROP (e.g., ED-ROP) of crosslinked P3 (containing PTSA) were evaluated.

For RCD, an oven-dried round bottomed flask equipped with a magnetic stir bar was charged with crosslinked P3 (1.0 wt %), dry toluene, zinc triflate (0.1 wt %), and activated molecular sieves (3 Å). The reaction mixture was then degassed and heated to reflux under a continuous nitrogen atmosphere for 24 hours. The reaction was cooled to rt, filtered to remove the catalyst and molecular sieves, and concentrated to remove toluene. The resulting solid was dissolved in CHCl₃ and partitioned with a large amount of water (to quench the remaining catalyst). The aqueous layer was extracted with CHCl₃ (3×). The combined organic layers were washed with water and brine, then dried over Na₂SO₄, filtered, and concentrated. The resulting sticky solid product was dried in vacuum. The yield (by weight) was 90%.

For ROP, the RCD mixture (PTSA catalyzed) was used. In a scintillation vial, the crosslinked P3 RCD mixture (22 wt. %) was fully dissolved in CDCl₃. To the reaction mixture, PTSA (0.5 wt. %) was added and stirred at rt. Within 2 hours, the reaction mixture gelled completely. This cross-linked polymer swelled in chloroform and tetrahydrofuran. The soluble fraction of the cross-linked network was analyzed by ¹H NMR (CDCl₃) and GPC (THF).

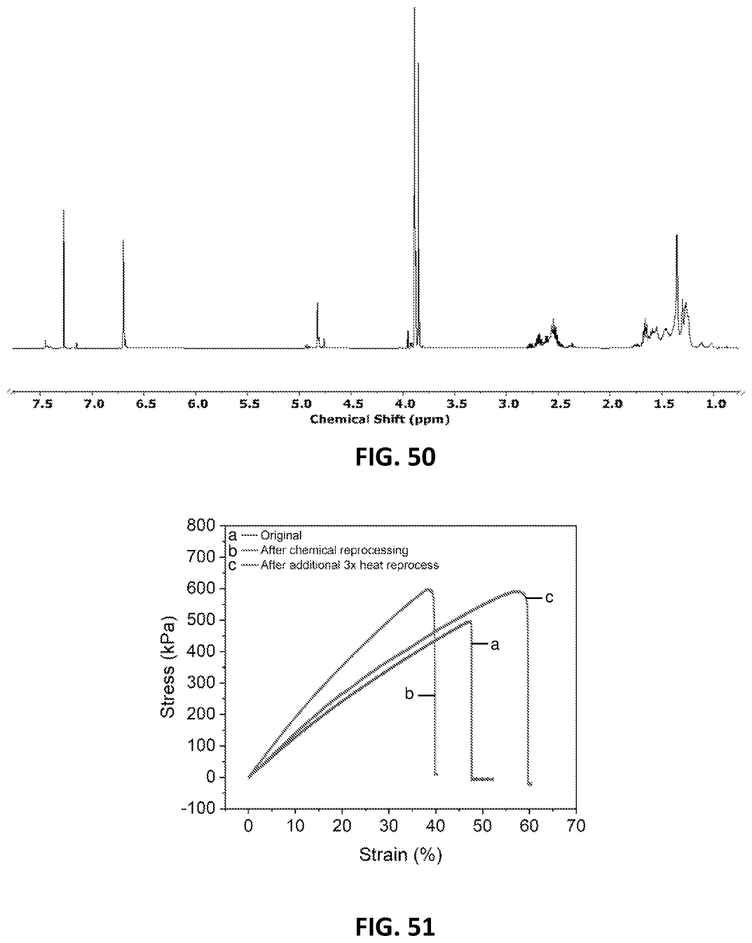

Depolymerization was evaluated using a concentration of 1.0 wt. % with zinc(II) triflate (0.1 wt. %) as the catalyst in refluxing toluene for 24 hours resulted in macrocycles as a solid in 90% yield by weight after purification to remove the catalyst (FIGS. 47A-47D and 48). The depolymerized mixture could be repolymerized into a gel (22 wt. %) within 2 hours in chloroform with PTSA (0.5 wt. %) as the catalyst. To avoid the change of catalyst during RCD and ROP, the chemical recyclability of a crosslinked P3 for which zinc(II) triflate was used to catalyze the initial synthesis was evaluated and found to remain in the original network instead of PTSA. Refluxing in toluene (1.1 wt. % polymer) without adding any additional catalysts for 24 hours yielded a homogeneous solution. After quick removal of the toluene at 30° C. in rotovap, a white-yellowish sticky powder was obtained which was confirmed by ¹H NMR as macrocycles (FIG. 50). The powder was then directly dissolved in chloroform to reach a concentration of 9.8 wt. % without any additional treatment. Gelation occurred at room temperature overnight. After drying, the polymer was heat-pressed into a film. The crosslinked P3 synthesized using zinc(II) triflate catalyst was softer and the gelation was slower than with PTSA catalyst (FIG. 51). After the chemical recycling, Young's modulus and tensile stress both improved, but the tensile strain decreased. Without being limited to a single theory, it currently is believed that this may indicate the formation of different network structures from ROP compared to the original one formed from step-growth polymerization.

The activation energy for chain exchange is relatively low compared to other reported systems, leading to stress-relaxation observed at 25° C. Without being limited to a single theory, it is believed that activation energy may be increased for some aspects of the disclosure using benzaldehyde monomers with fewer electron-donating substituents to increase the energy of the thiocarbenium intermediate, or using more rigid backbones. The increased tensile strength and relaxation time over multiple cycles observed here may be overcome by minimizing the number of residual macrocycles and the selection of other catalysts.

### Example 13

In this example, reprocessing of the polymer from Example 13 was conducted. The used samples of x-P3 were cut into small pieces as shown in FIG. 45 and placed back into the mold originally used to form the network. Once again, the sample was treated at 100° C. for 20 min with 5 tons of applied pressure in the heat press. Testing samples were cut from the re-molded material and were subjected to shear rheological testing or tensile testing. Three heat reprocessing cycles on the chemically recycled film were performed. Softening as well as a significant increase of extensibility was observed (FIGS. 46A-46C).

Stress-Relaxation Measurements—The stress-relaxation of a material follows the Maxwell equation for viscoelasticity:

$$G ( t ) / {G}_{o} = {e}^{( - τ / {τ}^{⋀} 4 )}$$

where G(t) is the relaxation modulus at time t, Gₒ is the initial relaxation modulus, and τ* is the relaxation time constant. Gₒ was taken at t=0.15 s, as the rearrangements associated with small exchanges have already taken place and the threshold to long-range exchanges has yet to be crossed. The calculated τ* from data taken at 150, 120, 90, and 70° C. were plotted against the inverse of those temperatures, which follows an Arrhenius adjacent model:

$$ln ( {τ}^{*} ) = ln ( A ) + {E}_{A} / RT$$

where E_{A} is the activation energy required for exchange, R is the universal gas constant, T is the temperature in Kelvin, and A is a pre-exponential constant.

Tables 9 and 10 provide a summary of measured Young's moduli, strain at break, and stress at break for crosslinked P3 (Table 9) and measured relaxation times for crosslinked P3 (Tables 10A-10D). As can be seen from these tables, at 10× recycled the 70° C. tests took 3 hours to relax as far as in previous trials and the τ* values are much higher than in previous trials. At 25° C. the material relaxed 66.5% after 5 hours and has thus not been included in the calculations represented here as it did not relax far enough to determine its relaxation constant. FIG. 49 shows the DSC curves for recycled crosslinked P3 measured from the second heating cycle.

TABLE 9
Measured Young's moduli, strain at break, and stress at break for crosslinked P3.

| | Young's Modulus (kPa) | | | Tensile Strain (%) | | | Tensile Stress (kPa) | | |
|---|---|---|---|---|---|---|---|---|---|
| Times | Sample | Sample | Sample | Sample | Sample | Sample | Sample | Sample | Sample |
| reprocessed | 1 | 2 | 3 | 1 | 2 | 3 | 1 | 2 | 3 |
| Original | 26.7 | 28.0 | 30.5 | 23.9 | 22.2 | 15.9 | 487 | 490 | 384 |
| 2 | 29.3 | 30.8 | 29.3 | 23.8 | 26.7 | 23.6 | 516 | 619 | 515 |
| 5 | 29.3 | 27.6 | 24.9 | 32.2 | 33.9 | 25.8 | 626 | 667 | 446 |
| 10 | 24.8 | 22.8 | 26.7 | 41.0 | 51.4 | 46.7 | 689 | 701 | 779 |

TABLE 10A
Measured relaxation times for crosslinked P3.

| Original | | | |
|---|---|---|---|
| Temperature | τ* (s) | | |
| (° C.) | Sample 1 | Sample 3 | Sample 4 |
| 70 | 1101.24 | 1182.00 | 1020.96 |
| 90 | 149.77 | 65.49 | 141.40 |
| 120 | 14.59 | 7.67 | 14.03 |
| 150 | 2.44 | 1.42 | 2.47 |
| E_{A} (kJ/mol) | 31.61 | 33.99 | 31.17 |

TABLE 10B
Measured relaxation times for crosslinked P3.

| | |
|---|---|
| 2x Recycled | τ* (s) |

| Temperature (° C.) | Sample 1 | Sample 2 | Sample 3 |
|---|---|---|---|
| 70 | 864.60 | 1340.93 | 893.52 |
| 90 | 113.97 | 173.74 | 119.19 |
| 120 | 11.13 | 16.76 | 11.78 |
| 150 | 1.99 | 2.81 | 2.03 |
| E_{A} (kJ/mol) | 31.44 | 31.87 | 31.47 |

TABLE 10C
Measured relaxation times for crosslinked P3.

| | |
|---|---|
| 5x Recycled | τ* (s) |

| Temperature (° C.) | Sample 1 | Sample 2 | Sample 3 |
|---|---|---|---|
| 70 | 1058.66 | 1644.96 | 1335.05 |
| 90 | 138.54 | 207.59 | 171.08 |
| 120 | 13.06 | 18.86 | 15.83 |
| 150 | 2.16 | 2.96 | 2.58 |
| E_{A} (kJ/mol) | 32.05 | 32.67 | 32.32 |

TABLE 10D
Measured relaxation times for crosslinked P3.

| | |
|---|---|
| 10x Recycled | τ* (s) |

| Temperature (° C.) | Sample 1 | Sample 2 | Sample 3 |
|---|---|---|---|
| 70 | 9215.16 | 6122.55 | 9992.43 |
| 90 | 837.04 | 790.44 | 783.54 |
| 120 | 65.77 | 63.14 | 62.01 |
| 150 | 8.56 | 8.61 | 8.50 |
| E_{A} (kJ/mol) | 35.90 | 34.02 | 36.27 |

### Example 14

### Homopolymer Synthesis

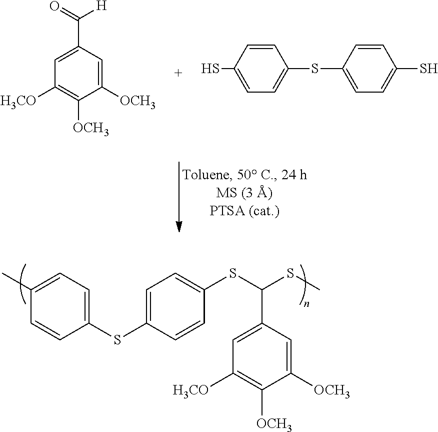

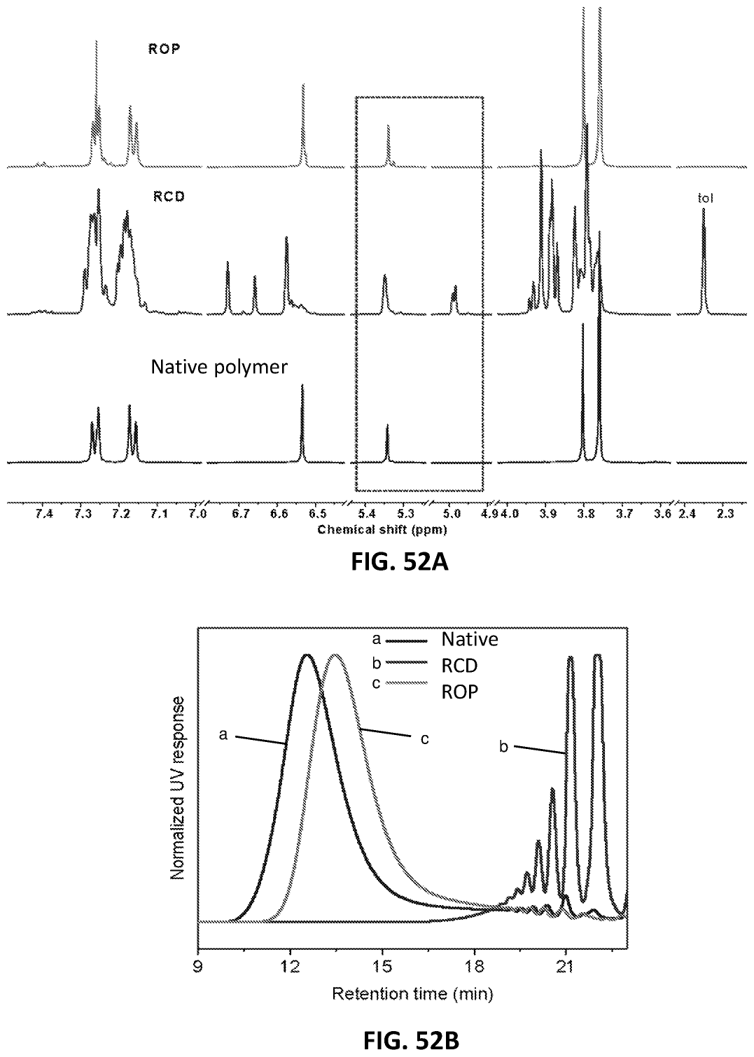

An oven-dried scintillation vial equipped with a magnetic stir bar was charged with 4,4′-thiobisbenzenethiol (638 mg, 2.50 mmol), 3,4,5-trimethoxybenzaldehyde (496 mg, 2.50 mmol), and toluene (1000 μL) such that the concentration of each of the two monomers were 2.5 M. The reaction mixture was placed in an oil bath preheated to 50° C. and let all the contents fully dissolved while equilibrating at 50° C. Activated molecular sieves (3 Å) were added and the reaction mixture was degassed. Para-toluenesulfonic acid monohydrate (PTSA) (5 mg, 0.03 mmol) was added, the mixture was purged with N₂ briefly, the vial was capped, let the reaction mixture stirred for 24 hours at 50° C. After 24 hours, the reaction was cooled to rt, triethylamine was added to quench the acid catalyst, fully dissolved in CHCl₃, and partitioned with saturated NaHCO₃ solution. The CHCl₃ layer was separated, and the aqueous layer was again extracted with additional CHCl₃. The combined organic layers were washed with water and brine, dried over anhydrous Na₂SO₄, filtered, and concentrated. The polymer was then purified by dissolving in CHCl₃ and precipitating with methanol. Characterization results are shown in FIGS. 52A and 52B.

### Copolymer Synthesis

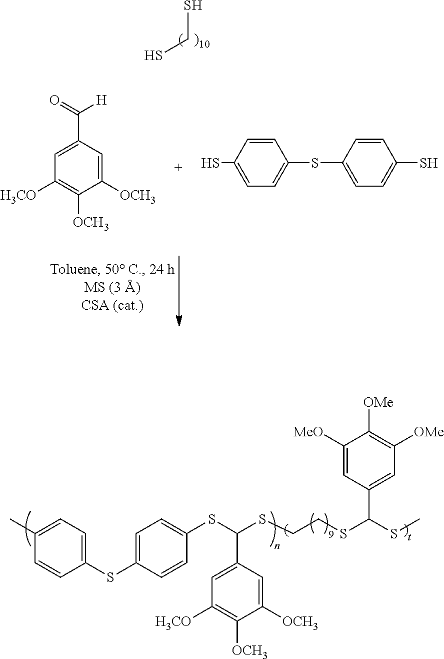

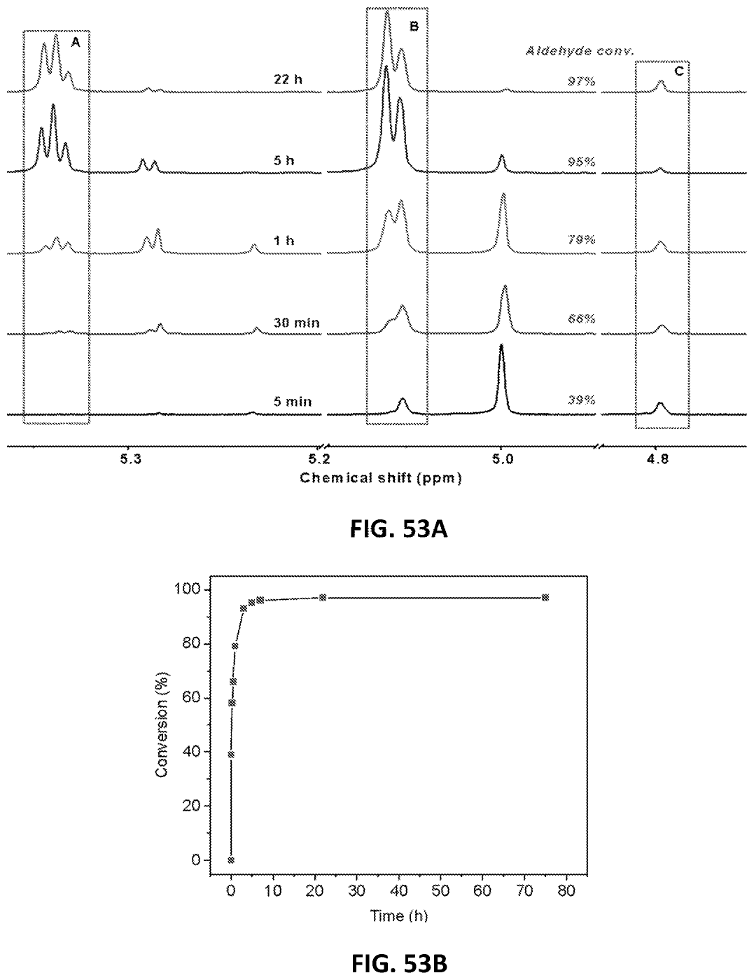

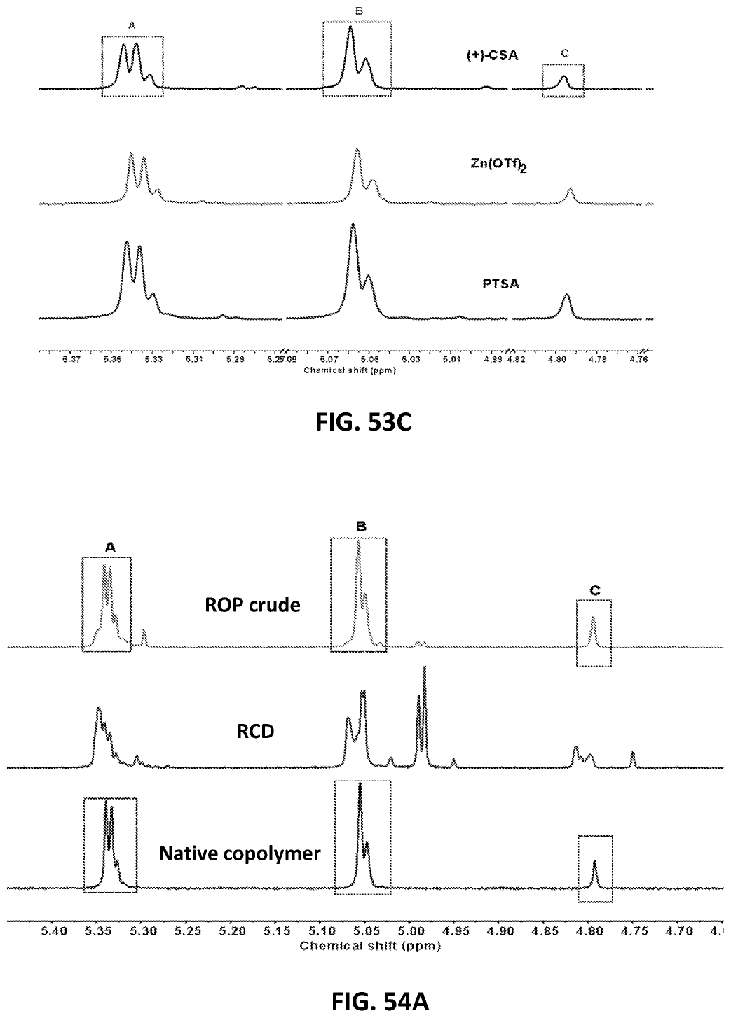

Synthesis of copolymer comprising 70% mol of aromatic and 30% mol of aliphatic dithiols in the polymer backbone: An oven-dried scintillation vial equipped with a magnetic stir bar was charged with 4,4′-thiobisbenzenethiol (894 mg, 3.50 mmol), 1,10-decanedithiol (310 mg, 1.50 mmol), 3,4,5-trimethoxybenzaldehyde (982 mg, 5.00 mmol), and toluene (1000 μL) such that [benzaldehyde]=[total dithiol]=5.0 M. The reaction mixture was placed in an oil bath preheated to 50° C. and let all the contents fully dissolved while equilibrating at 50° C. Activated molecular sieves (3 Å) were added, and the reaction mixture was degassed. Subsequently, racemic-10-camphorsulfonic acid (CSA) (14 mg, 0.06 mmol) was added, the mixture was purged with N₂, the vial was capped, and the reaction mixture was stirred for 24 hours at 50° C. After 24 hours, the reaction was cooled to rt, triethylamine was added to quench the acid catalyst, fully dissolved in CHCl₃. Lastly, the mixtures was partitioned with a saturated NaHCO₃ solution. The CHCl₃ layer was separated, and the aqueous layer was extracted one more time with additional CHCl₃, the combined organic layers were washed with water and brine, dried over anhydrous Na₂SO₄, filtered, and concentrated. The polymer was then purified by dissolving in CHCl₃ and precipitating with methanol. Characterization results are shown in FIGS. 53A and 53B. Mₙ and Ð values in some examples were 25 kDa and 5.04, respectively.

The method was repeated with different acid catalysts (PTSA, Zn(OTf)₂, and (+)-CSA. ¹H NMR and GPC results are shown in FIG. 53C. Peak integration values are provided in Table 11.

TABLE 11

| | | | |
|---|---|---|---|
| Catalyst | Rel Int [A] | Rel Int [B] | Rel Int [C] |

| | | | | | |
|---|---|---|---|---|---|
| PTSA | 6.78 | (48%) | 6.44 | (45%) | 1.00 |
| Zn(OTf)₂ | 6.99 | (48%) | 6.58 | (45%) | 1.00 |
| (+)-CSA | 7.61 | (46%) | 8.07 | (48%) | 1.00 |
| rac-CSA | 11.05 | (43%) | 13.42 | (53%) | 1.00 |

In the same way, copolymers comprising 50% mol of aromatic and 50% aliphatic dithiols as well as 30% mol of aromatic and 70% aliphatic dithiols in the polymer backbone were synthesized.

### Example 15

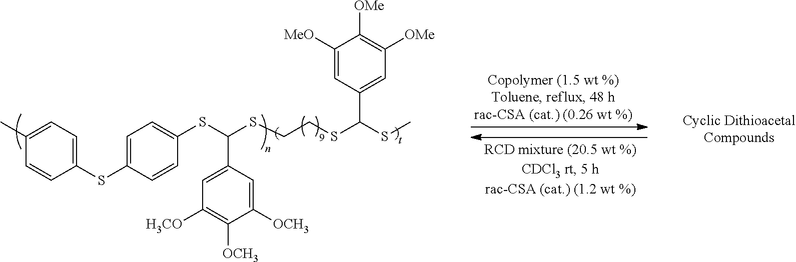

### Ring-Closing Depolymerization (RCD):

Depolymerization of the aryl homopolymer: In oven-dried round bottomed flask equipped with a magnetic stir bar was charged with purified polymer (580 mg, 1.5 wt %), toluene (44 mL), and rac-10-camphorsulfonic acid (99 mg, 0.3 wt %) as the catalyst. The polymer was fully dissolved. The reaction mixture was then degassed and heated to reflux under a continuous nitrogen atmosphere overnight. The reaction was cooled to rt, partitioned with saturated NaHCO₃ solution, toluene layer separated, and concentrated. The resulting solid was dissolved in CHCl₃, partitioned with brine, CHCl₃ layer separated, then dried over Na₂SO₄, filtered, and concentrated. A mixture of dithioacetal macrocycles were obtained as a powder.

Depolymerization of copolymers: The same procedure as outlined above for the homopolymer was carried out except for a longer reaction time of 2 days.

### Entropy-Driven Ring-Opening Polymerization (ED-ROP)

ED-ROP to form aryl homopolymer: In a scintillation vial, the RCD macrocyclic mixture (470 mg, 14.8 wt. %) was dissolved in CDCl₃ (1800 μL). Rac-10-camphorsulfonic acid (5 mg, 0.16 wt. %) was added, and stirred at 50° C. overnight. The entire reaction was quenched with triethylamine, diluted with CHCl₃, and partitioned with saturated NaHCO₃ solution. The aqueous layer was extracted with CHCl₃ (2×). The combined organic layers were washed with water and brine, dried over Na₂SO₄, filtered, concentrated.

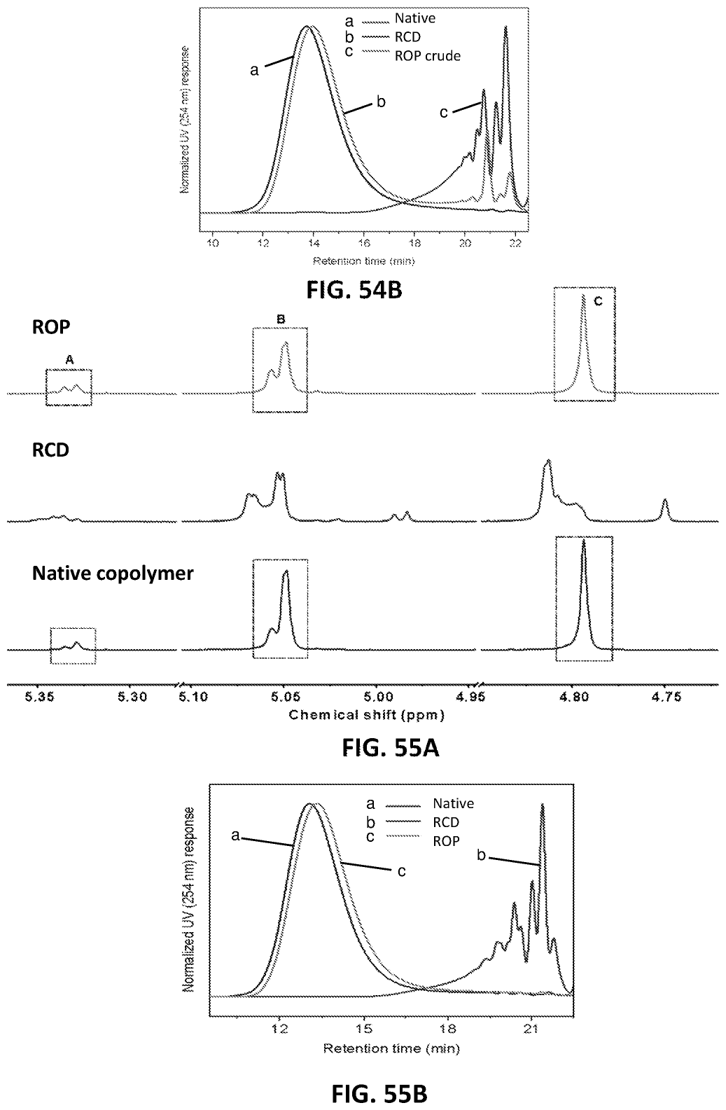

ED-ROP to form aryl copolymer: Same procedure was carried out for the ROP of macrocyclic mixtures from the RCD of copolymers. For example, 30% mol aliphatic and 70% mol aromatic dithiol comprising copolymer derived macrocyclic mixture (447 mg, 22.9 wt. %) in CDCl₃ (1000 μL) with rac-10-camphorsulfonic acid (5 mg, 0.26 wt. %) was heated to 50° C. overnight. Characterization results are shown in FIGS. 54A and 54B. Peak integration values are provided in Table 12. Mₙ and Ð values for the native linear polymer were 21 kDa and 3.67, respectively. Mₙ and Ð values for the recycled polymer after ROP were 21 kDa and 3.58, respectively.

TABLE 12

| | Rel Int [A] | Rel Int [B] | Rel Int [C] |
|---|---|---|---|
| Native linear polymer | 7.19 (48%) | 6.81 (45%) | 1.00 (6%) |
| ROP crude | 6.70 (48%) | 6.16 (44%) | 1.00 (7%) |

In yet another example, 70% mol aliphatic and 30% mol aromatic dithiol comprising copolymer derived macrocyclic mixture (447 mg, 22.9 wt. %) in CDCl₃ (1000 μL) with rac-10-camphorsulfonic acid (5 mg, 0.26 wt. %) was heated to 50° C. overnight. Characterization results are shown in FIGS. 55A and 55B. Peak integration values are provided in Table 13. Mₙ and Ð values for the native linear polymer were 52 kDa and 3.00, respectively. Mₙ and Ð values for the recycled polymer after ROP were 43 kDa and 2.98, respectively.

TABLE 13

| | Rel Int [A] | Rel Int [B] | Rel Int [C] |
|---|---|---|---|
| Native linear polymer | 0.08 (4%) | 1.11 (51%) | 1.00 (46%) |
| ROP | 0.14 (7%) | 0.89 (44%) | 1.00 (49%) |

### Example 16

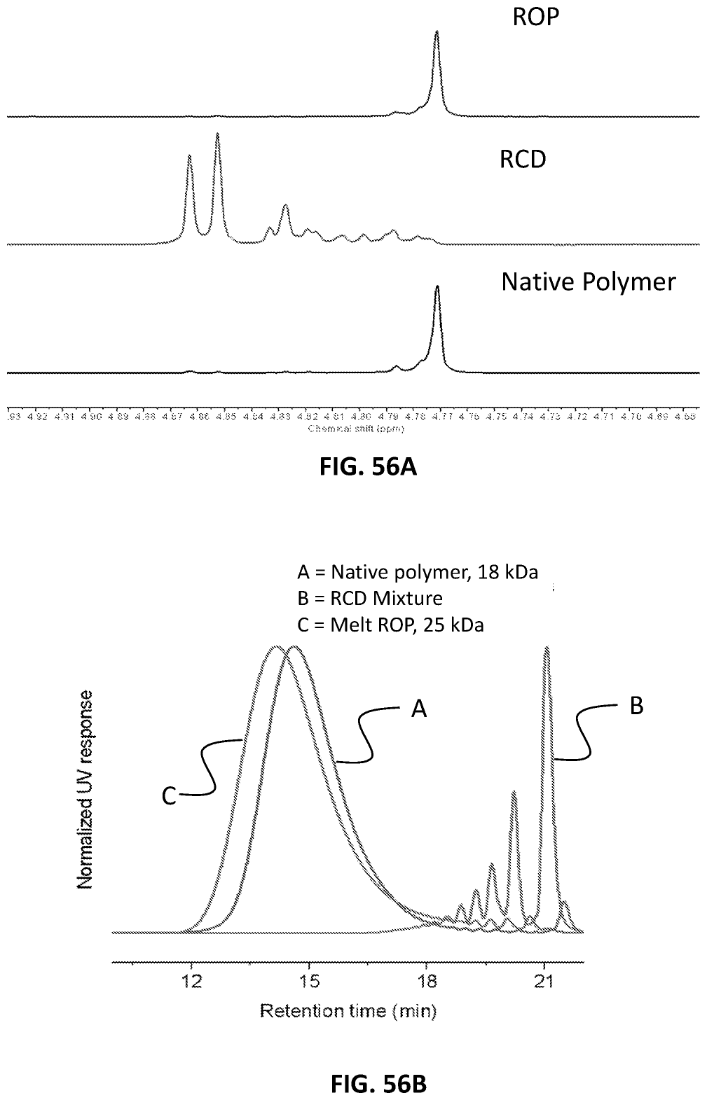

In this example, melt polymerization was used to convert a cyclic dithioacetal compound to a recycled polymer. A scintillation vial equipped with a magnetic stir bar was charged with the RCD mixture derived from native polymer P1 (80 mg, 98 wt %) and heated to 120° C. After the macrocycles were melted, rac-CSA (1.5 mg, 2 wt %) was added to the reaction and heating was continued for 1.5 hours. The reaction was quenched with triethylamine prior to analysis by NMR (FIG. 56A) and GPC (FIG. 56B).

In view of the many possible embodiments to which the principles of the present disclosure may be applied, it should be recognized that the illustrated embodiments are only preferred examples and should not be taken as limiting the scope of the disclosure. Rather, the scope of the present disclosure is defined by the following claims. We therefore claim as our invention all that comes within the scope and spirit of these claims.

1. A linear polymer having a structure according to Formula I, 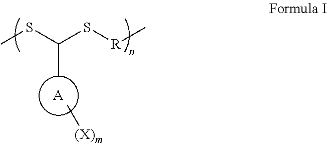
wherein
ring A is an aromatic ring system;
each R independently is selected from aliphatic, aromatic, heteroaliphatic, or any combination thereof; or
each R independently is selected from an R′ group, wherein
R′ has a structure according to a Formula A, —R^{c}—S—R^{d}—S—R^{c}—, wherein each R^{c} is selected from the groups recited for R, and R^{d} is a group having a Formula B, —R^{c}—S[CH₂]_{q}O—Ar—C(Me₂)-Ar—O[CH₂]_{q}S—R^{c}—, or a Formula C, —R^{c}—S[CH₂]_{q}Y[CH₂]_{q}S—R^{c}—, wherein for Formula B, each Ar is an aromatic ring system; for both Formulas B and C, each q independently is an integer selected from 1 to 10; and for Formula C, Y is selected from an amide group, a carbamide group, or a sulfonyl group;
each X independently is selected from an electron-withdrawing group or an electron-donating group;
n is an integer selected to provide an Mₙ ranging from 5,000 g/mol to 120,000 g/mol; and
m is an integer ranging from 0 to 5; provided that
(i) if ring A is phenyl and m is 0, then R is not —(CH₂)₆—, —(CH₂CH₂O)₂CH₂CH₂—, -Ph-S-Ph-;
(ii) if ring A is phenyl, m is 1, and X is para-methoxy, then R is not —(CH₂)₆—, —(CH₂CH₂O)₂CH₂CH₂—-Ph-S-Ph-, —CH₂C(O)O(CH₂)₄OC(O)CH₂—, or —(CH₂)₂C(O)O(CH₂)₄OC(O)(CH₂)₂—;
(iii) if ring A is phenyl, m is 1, and X is para-OCH₂CH═CH₂, then R is not —(CH₂CH₂O)₂CH₂CH₂—;
(iv) if ring A is phenyl, m is 1, and X is para-OH, then R is not —(CH₂CH₂O)₂CH₂CH₂—;
(v) if ring A is phenyl, m is 1, and X is para-Cl, then R is not —(CH₂CH₂O)₂CH₂CH₂—; and/or
(vi) if ring A is phenyl, m is 1, and X is para-NO₂, then R is not —(CH₂CH₂O)₂CH₂CH₂—.

2. The linear polymer of claim 1, wherein:
ring A is an aryl or heteroaryl ring system;
each R independently is selected from alkyl, alkenyl, alkynyl, aryl, heteroaryl, heteroalkyl, heteroalkenyl, heteroalkynyl, or any combination thereof;
each X independently is selected from alkyl, alkenyl, alkynyl, aryl, heteroaryl, heteroalkyl, heteroalkenyl, heteroalkynyl, haloalkyl, haloalkenyl, haloalkynyl, or any combination thereof, or Br, F, I, Cl, nitro, cyano, hydroxyl, thiol, or —NH₂;
n is an integer selected to provide an Mₙ ranging from 5,000 g/mol to 120,000 g/mol; and
m is an integer ranging from 0 to 5.

3. The linear polymer of claim 1, wherein:
ring A is phenyl, naphthyl, thiophene, furan, pyrrole, or indolyl;
each R independently is selected from C₆-C₁₀ alkyl; ether; thioether; aryl; biaryl; triaryl; -Ph-Z-Ph- wherein Z is oxygen, sulfur, or NH; —CH₂PhCH₂—; —CH₂C(O)O(CH₂)ᵣO(O)CCH₂—, wherein r is an integer ranging from 1 to 10; or
each R independently is —R^{c}—S[CH₂]_{q}O-Ph-C(Me₂)-Ph-O[CH₂]_{q}S—R^{c}— or —R^{c}—S[CH₂]_{q}Y[CH₂]_{q}S—R^{c}— wherein
each R^{c} is C₆-C₁₀ alkyl; ether; thioether; aryl; biaryl; triaryl; -Ph-Z-Ph-, wherein Z is oxygen, sulfur, or NH; —CH₂PhCH₂—, or —CH₂C(O)O(CH₂)ᵣO(O)CCH₂—, wherein r is an integer ranging from 1 to 10; and each q is an integer selected from 1 to 4; and Y is carbamide, amide, or sulfonyl;
each X independently is selected from alkyl, alkenyl, alkynyl, aryl, heteroaryl, heteroalkyl, heteroalkenyl, heteroalkynyl, haloalkyl, haloalkenyl, haloalkynyl, or any combination thereof, or Br, F, I, Cl, nitro, cyano, hydroxyl, thiol, or —NH₂;
n is an integer selected to provide an Mₙ ranging from 10,000 g/mol to 120,000 g/mol; and
m is an integer ranging from 0 to 3.

4. The linear polymer of claim 1, wherein:
ring A is phenyl, thiophene, furan, or pyrrole;
each R independently is selected from C₆ alkyl, C₈ alkyl, C₁₀ alkyl, —[CH₂]₂O[CH₂]₂—, —[CH₂]₂S[CH₂]₂—, —[CH₂]₂[OCH₂]₂—, —[CH₂]₂[SCH₂]₂—, phenyl, Ph-C(O)OH, biphenyl, triphenyl, -Ph-O-Ph-, -Ph-S- Ph-, —CH₂PhCH₂—, —CH₂C(O)O(CH₂)₄O(O)CCH₂—; or
each R independently is selected from —R^{c}—S[CH₂]₂O-Ph-C(Me₂)-Ph-O[CH₂]₂S—R^{c}—, —R^{c}—S[CH₂]₂C(O)NH[CH₂]₃S—R^{c}—, —R^{c}—S[CH₂]₂NHC(O)NH[CH₂]₂S—R^{c}—, or —R^{c}—S[CH₂]₂SO₂[CH₂]₂S—R^{c}—, wherein
each R^{c} is C₆ alkyl, C₈ alkyl, C₁₀ alkyl, —[CH₂]₂O[CH₂]₂—, —[CH₂]₂S[CH₂]₂—, —[CH₂]₂[OCH₂]₂—, —[CH₂]₂[SCH₂]₂—, phenyl, biphenyl, triphenyl, -Ph-O-Ph-, -Ph-S-Ph-, —CH₂PhCH₂—, or —CH₂C(O)O(CH₂)₄O(O)CCH₂—);
each X independently is selected from methyl; ethyl; propyl and isomers thereof; butyl and isomers thereof; pentyl and isomers thereof; hexyl and isomers thereof; phenyl; —CH═CHPh; —OPh-C(O)H;
alkoxy; amine; —N(H)C(O)CH₃; —OC(O)R″, wherein R″ is hydrogen, aliphatic, aromatic, or heteroaliphatic; H₃C[O(CH₂)₂]ₛO—, wherein s is an integer selected from 1 to 10; Br; F; I; Cl; CF₃; nitro; cyano; hydroxyl; thiol; —NH₂; —C(O)OR″, wherein R″ is hydrogen, aliphatic, aromatic, or heteroaliphatic; or —C(O)H;
n is an integer ranging from 2 to 200; and
m is 0, 1, 2, or 3.

5. The linear polymer of claim 1 having a structure according to Formula IA, IB, IC, or ID 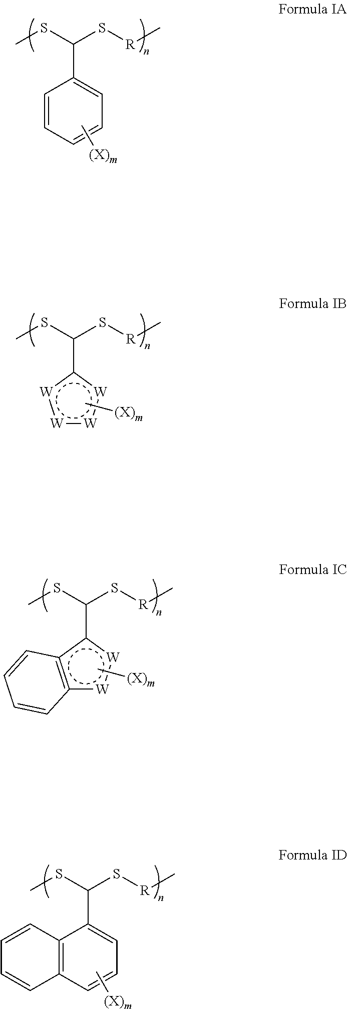
wherein each W independently is selected from —CH, oxygen, NH, NX, or S.

6. The linear polymer of claim 1, wherein the linear polymer is a native linear polymer obtained from reacting a dithiol monomer component and an aldehyde-containing monomer compound; or wherein the linear polymer is a recycled linear polymer obtained from ring-closing depolymerization of a native linear polymer to a cyclic dithioacetal compound and subsequent ring-opening polymerization of the cyclic dithioacetal compound.

7. (canceled)

8. The linear polymer of claim 1, selected from 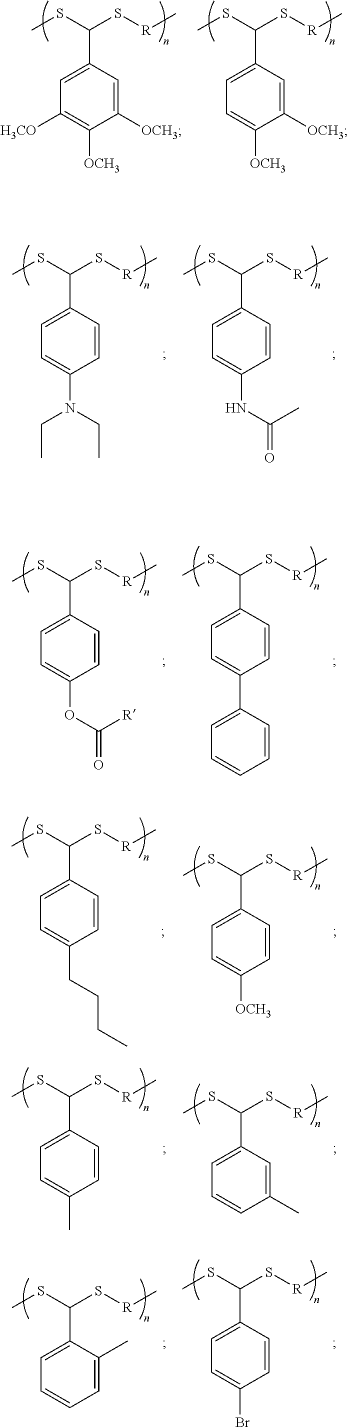 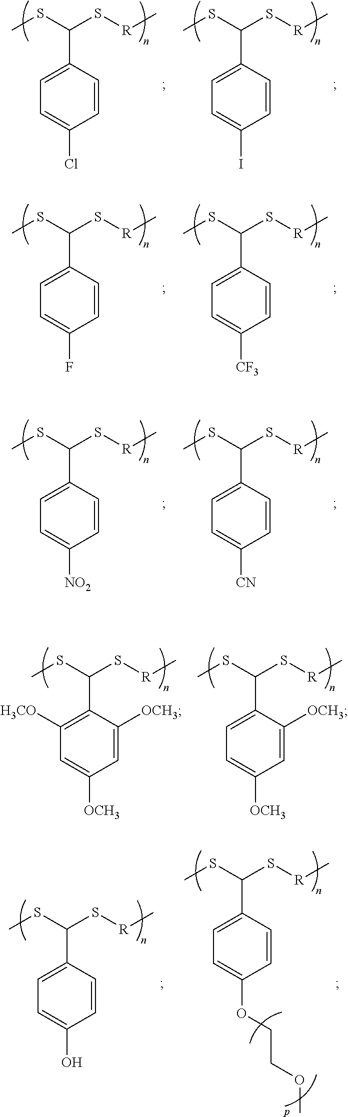 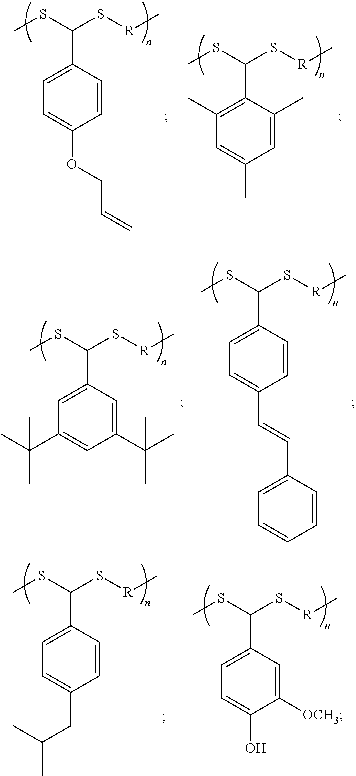 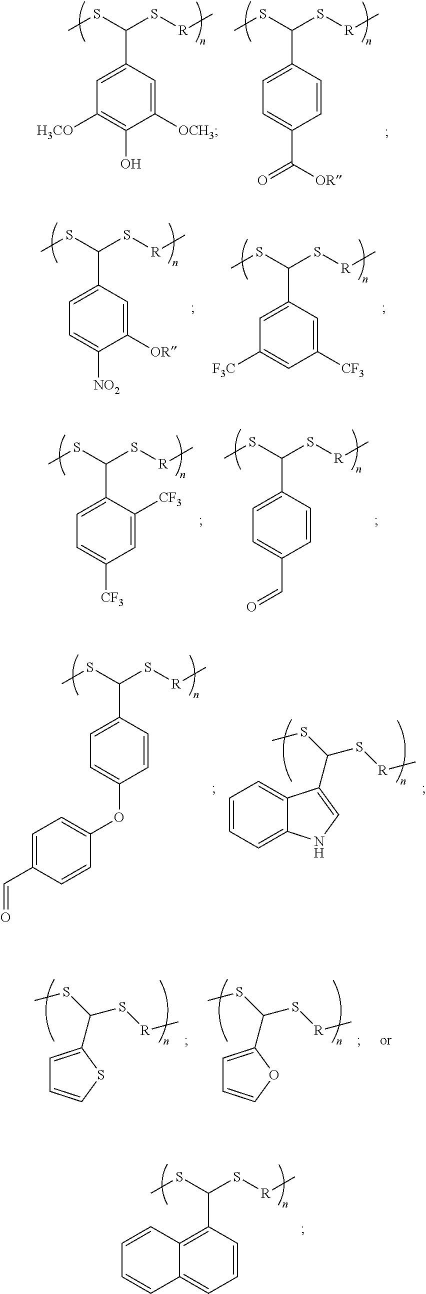
and
wherein each R independently is selected from

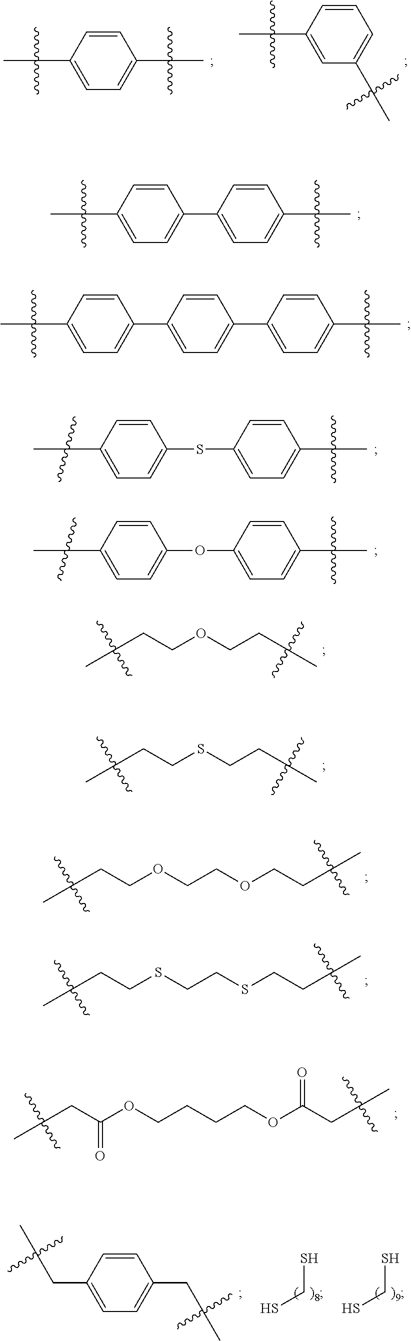

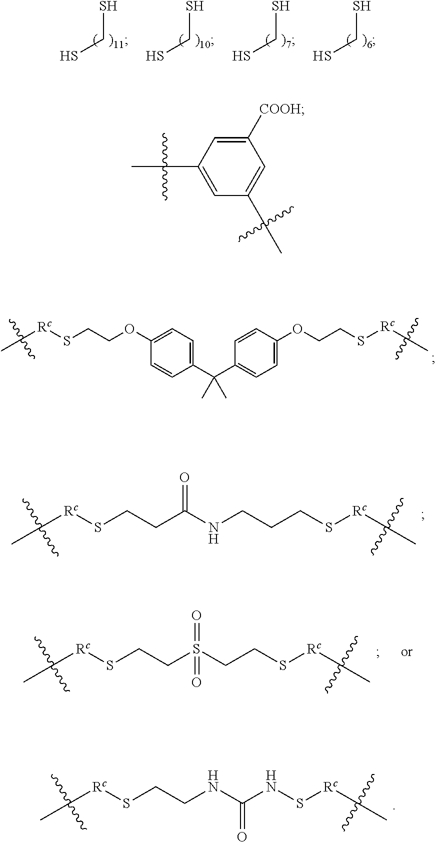

9. (canceled)

10. The linear polymer of claim 1, selected from: 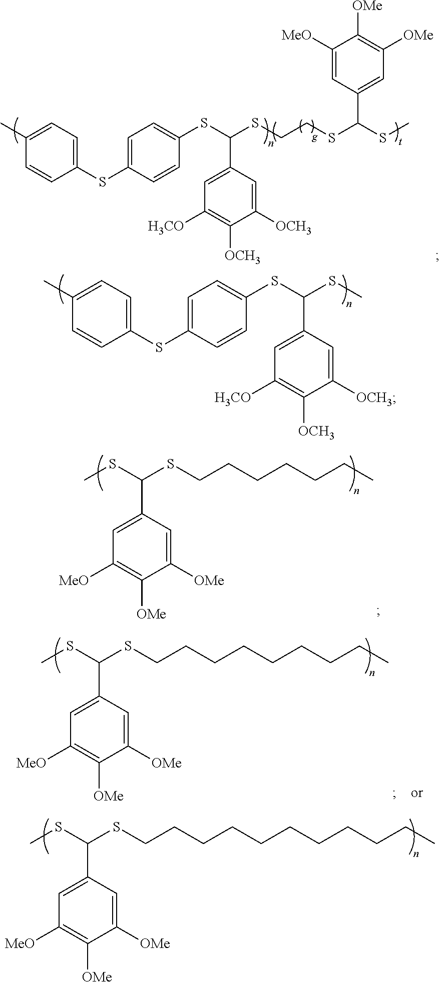
wherein t is selected from values for n and wherein t is the same or different as n.

11. A cyclic dithioacetal compound having a structure according to Formula II 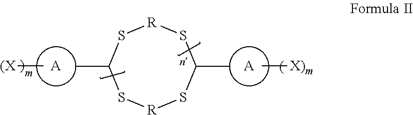
wherein:
ring A is an aromatic ring system;
each R independently is selected from aliphatic, aromatic, heteroaliphatic, or any combination thereof; or
each R independently is selected from an R′ group, wherein
R′ has a structure according to a Formula A, —R^{c}—S—R^{d}—S—R^{c}—, wherein each R^{c} is selected from the groups recited for R, and R^{d} is a group having a Formula B, —R^{c}—S[CH₂]_{q}O—Ar—C(Me₂)-Ar—O[CH₂]_{q}S—R^{c}—, or a Formula C, —R^{c}—S[CH₂]_{q}Y[CH₂]_{q}S—R^{c}—, wherein for Formula B, each Ar is an aromatic ring system; for both Formulas B and C, each q independently is an integer selected from 1 to 10; and for Formula C, Y is selected from an amide group, a carbamide group, or a sulfonyl group;
each X independently is selected from an electron-withdrawing group or an electron-donating group;
n′ is an integer ranging from 1 to 8; and
m is an integer ranging from 0 to 5; provided that
(i) if ring A is phenyl and m is 0, then R is not —(CH₂)₆—, —(CH₂CH₂O)₂CH₂CH₂—, -Ph-S-Ph-;
(ii) if ring A is phenyl, m is 1, and X is para-methoxy, then R is not —(CH₂)₆—, —(CH₂CH₂O)₂CH₂CH₂—-Ph-S-Ph-, —CH₂C(O)O(CH₂)₄OC(O)CH₂—, or —(CH₂)₂C(O)O(CH₂)₄OC(O)(CH₂)₂—;
(iii) if ring A is phenyl, m is 1, and X is para-OCH₂CH═CH₂, then R is not —(CH₂CH₂O)₂CH₂CH₂—;
(iv) if ring A is phenyl, m is 1, and X is para-OH, then R is not —(CH₂CH₂O)₂CH₂CH₂—;
(v) if ring A is phenyl, m is 1, and X is para-Cl, then R is not —(CH₂CH₂O)₂CH₂CH₂—; and/or
(vi) if ring A is phenyl, m is 1, and X is para-NO₂, then R is not —(CH₂CH₂O)₂CH₂CH₂—.

12. The cyclic dithioacetal compound of claim 11, wherein:
ring A is an aryl or heteroaryl ring system;
each R independently is selected from alkyl, alkenyl, alkynyl, aryl, heteroaryl, heteroalkyl, heteroalkenyl, heteroalkynyl, or any combination thereof;
each X independently is selected from alkyl, alkenyl, alkynyl, aryl, heteroaryl, heteroalkyl, heteroalkenyl, heteroalkynyl, haloalkyl, haloalkenyl, haloalkynyl, or any combination thereof, or Br, F, I, Cl, nitro, cyano, hydroxyl, thiol, or —NH₂;
n′ is an integer ranging from 1 to 6; and
m is an integer ranging from 0 to 5.

13. The cyclic dithioacetal compound of claim 11, wherein:
ring A is phenyl, naphthyl, thiophene, furan, pyrrole, or indolyl;
each R independently is selected from C₆-C₁₀ alkyl; ether; thioether; aryl; biaryl; triaryl; -Ph-Z-Ph- wherein Z is oxygen, sulfur, or NH; —CH₂PhCH₂—; —CH₂C(O)O(CH₂)ᵣO(O)CCH₂—, wherein r is an integer ranging from 1 to 10; or
each R independently is —R^{c}—S[CH₂]_{q}O-Ph-C(Me₂)-Ph-O[CH₂]_{q}S—R^{c}— or —R^{c}—S[CH₂]_{q}Y[CH₂]_{q}S—R^{c}— wherein
each R^{c} is C₆-C₁₀ alkyl; ether; thioether; aryl; biaryl; triaryl; -Ph-Z-Ph-, wherein Z is oxygen, sulfur, or NH; —CH₂PhCH₂—, or —CH₂C(O)O(CH₂)ᵣO(O)CCH₂—, wherein r is an integer ranging from 1 to 10; and each q is an integer selected from 1 to 4; and Y is carbamide, amide, or sulfonyl;
each X independently is selected from alkyl, alkenyl, alkynyl, aryl, heteroaryl, heteroalkyl, heteroalkenyl, heteroalkynyl, haloalkyl, haloalkenyl, haloalkynyl, or any combination thereof, or Br, F, I, Cl, nitro, cyano, hydroxyl, thiol, or —NH₂;
n′ is an integer ranging from 1 to 4; and
m is an integer ranging from 0 to 3.

14. The cyclic dithioacetal compound of claim 11, wherein:
ring A is phenyl, thiophene, furan, or pyrrole;
each R independently is selected from C₆ alkyl, C₈ alkyl, C₁₀ alkyl, —[CH₂]₂O[CH₂]₂—, —[CH₂]₂S[CH₂]₂—, —[CH₂]₂[OCH₂]₂—, —[CH₂]₂[SCH₂]₂—, phenyl, Ph-C(O)OH, biphenyl, triphenyl, -Ph-O-Ph-, -Ph-S- Ph-, —CH₂PhCH₂—, —CH₂C(O)O(CH₂)₄O(O)CCH₂—; or
each R independently is selected from —R^{c}—S[CH₂]₂O-Ph-C(Me₂)-Ph-O[CH₂]₂S—R^{c}—, —R^{c}—S[CH₂]₂C(O)NH[CH₂]₃S—R^{c}—, —R^{c}—S[CH₂]₂NHC(O)NH[CH₂]₂S—R^{c}—, or —R^{c}—S[CH₂]₂SO₂[CH₂]₂S—R^{c}—, wherein
each R^{c} is C₆ alkyl, C₈ alkyl, C₁₀ alkyl, —[CH₂]₂O[CH₂]₂—, —[CH₂]₂S[CH₂]₂—, —[CH₂]₂[OCH₂]₂—, —[CH₂]₂[SCH₂]₂—, phenyl, biphenyl, triphenyl, -Ph-O-Ph-, -Ph-S-Ph-, —CH₂PhCH₂—, or —CH₂C(O)O(CH₂)₄O(O)CCH₂—);
each X independently is selected from methyl; ethyl; propyl and isomers thereof; butyl and isomers thereof; pentyl and isomers thereof; hexyl and isomers thereof; phenyl; —CH═CHPh; —OPh-C(O)H; alkoxy; amine; —N(H)C(O)CH₃; —OC(O)R″, wherein R″ is hydrogen, aliphatic, aromatic, or heteroaliphatic; H₃C[O(CH₂)₂]ₛO—, wherein s is an integer selected from 1 to 10; Br; F; I; Cl; CF₃; nitro; cyano; hydroxyl; thiol; —NH₂; —C(O)OR″, wherein R″ is hydrogen, aliphatic, aromatic, or heteroaliphatic; or —C(O)H;
n′ is an integer ranging from 1 to 4; and
m is 0, 1, 2, or 3.

15. The cyclic dithioacetal compound of claim 11, having a structure according to Formula IIA, IIB, IIC, or IID 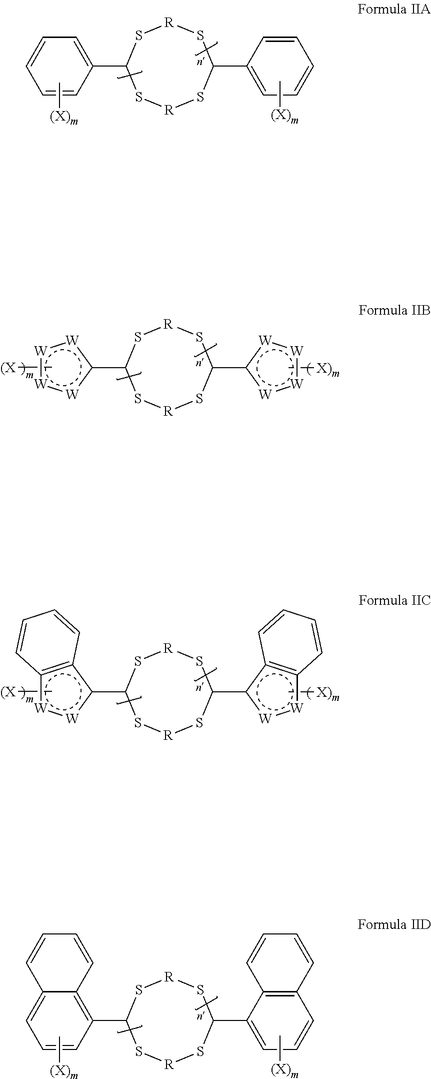
wherein each W independently is selected from —CH, oxygen, NH, NX, or S.

16. The cyclic dithioacetal compound of claim 11, selected from 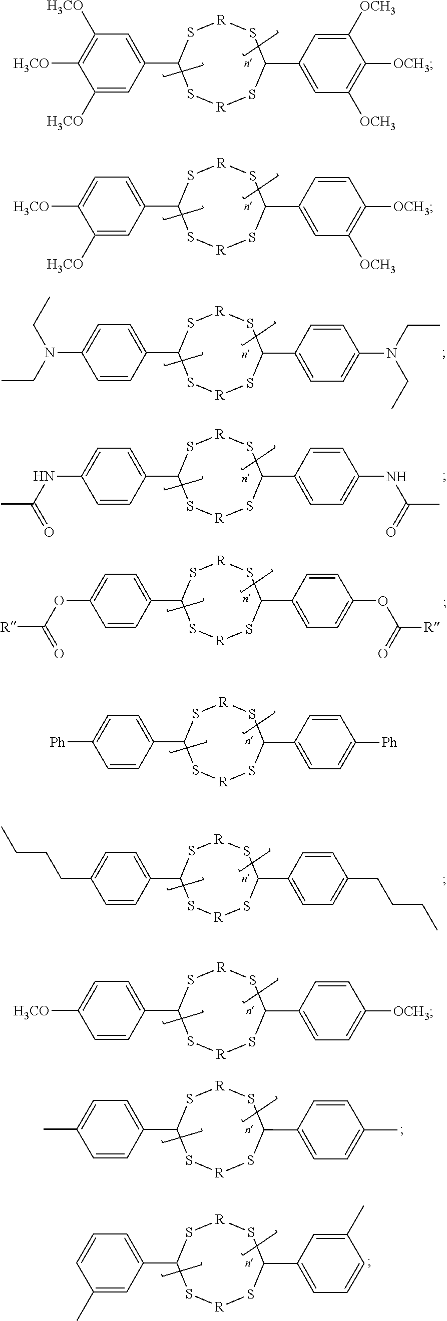 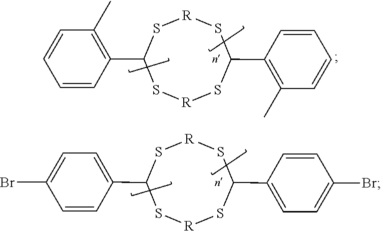 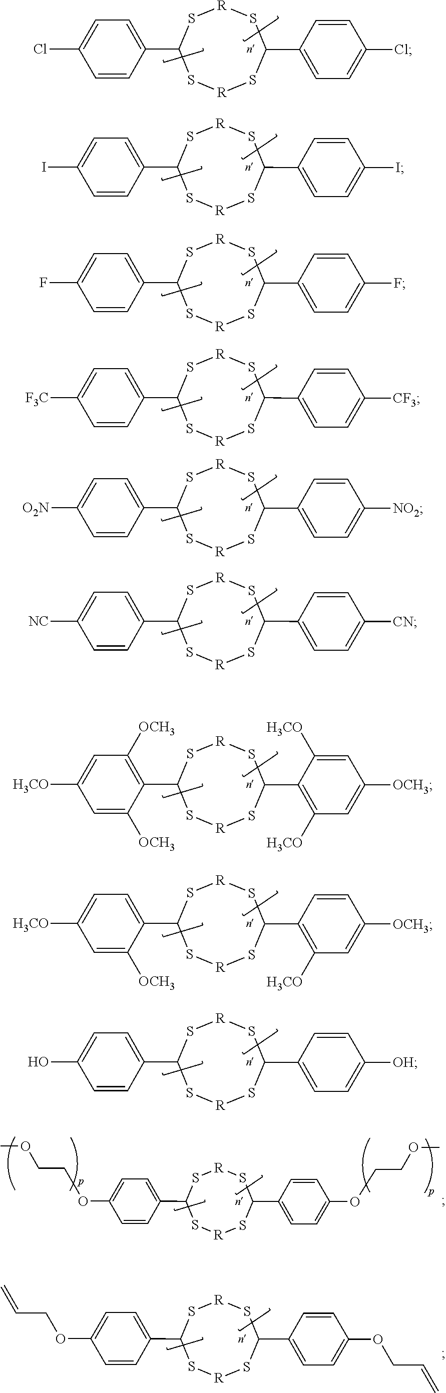 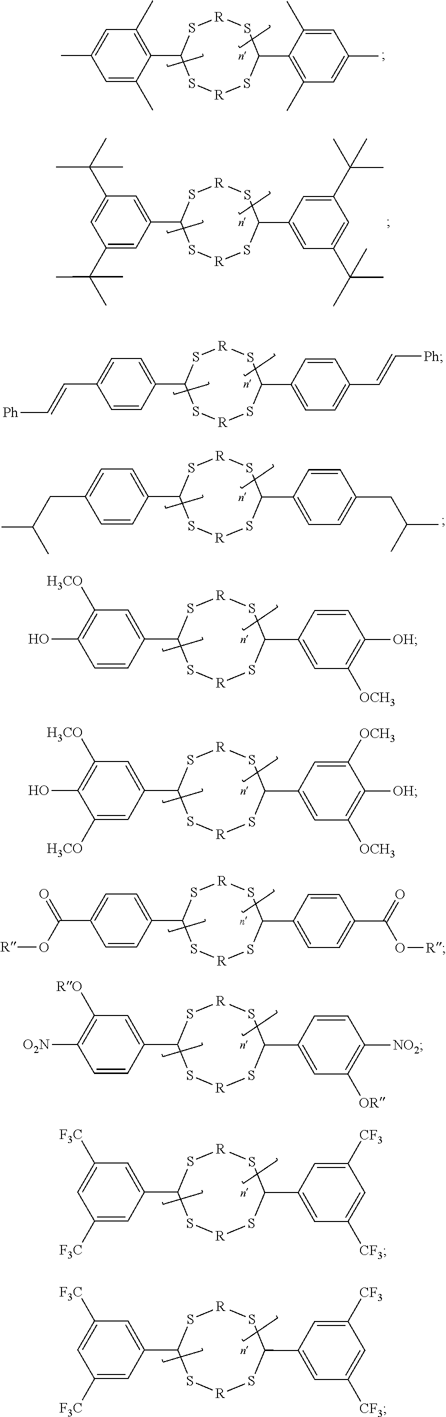 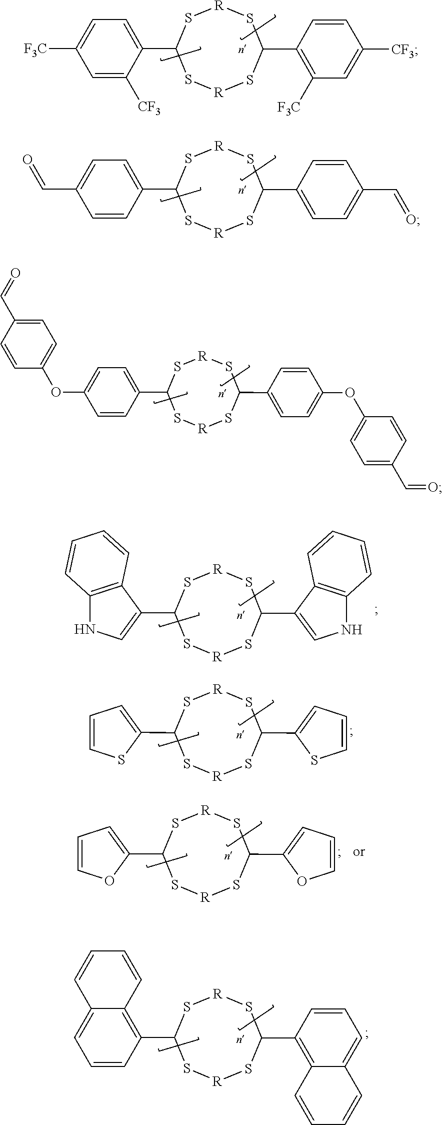
and
wherein each R independently is selected from

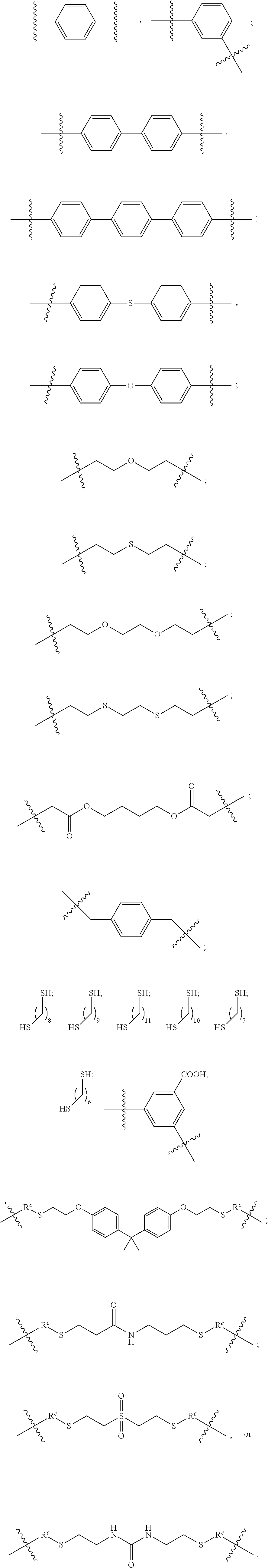

17. (canceled)

18. The cyclic dithioacetal compound of claim 11, having a structure according to a formula 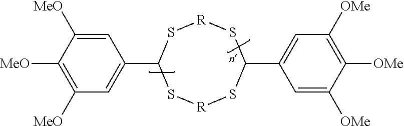
wherein R is (CH₂)₆, (CH₂)₈, or (CH₂)₁₀ and n′ is an integer ranging from 1 to 8; or wherein a combination of two different R groups is present and wherein at least one R is -Ph-S-Ph- and at least one R is (CH₂)₆, (CH₂)₈, or (CH₂)₁₀.

19. A method for making the linear polymer of claim 1, comprising:
exposing a dithiol monomer component and an aldehyde-containing monomer component to an acid catalyst to form a reaction mixture; and
exposing the reaction mixture to a reaction temperature;
wherein
the dithiol monomer component has a structure according to Formula III 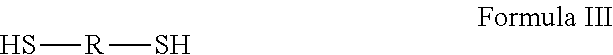
wherein R is as recited for claim 1; and
the aldehyde-containing monomer component has a structure according to Formula IV, wherein ring A, X, and m are as recited for claim 1

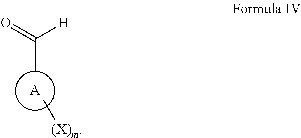

20. The method of claim 19, wherein the acid catalyst is selected from a Lewis acid catalyst or a Brønsted acid catalyst.

21. The method of claim 20, wherein the Lewis acid catalyst is selected from BF₃, BCl₃, FeCl₃, AlCl₃, GaCl₃, InCl₃, SbCl₃, SbCl₅, BiCl₃, TiCl₄, ZnCl₂, ZrCl₄, SnCl₄, HfCl₄, Zn(OTf)₂, Cu(OTf)₂, Sn(OTf)₂, or any combination thereof; or wherein the Brønsted acid catalyst is selected from p-TsOH, CF₃COOH, 10-camphorsulfonic acid, or any combination thereof.

22. (canceled)

23. The method of claim 19, wherein the reaction temperature ranges from ambient temperature to 80° C.

24. A method for making a cyclic dithioacetal compound, comprising:
exposing a polymer of claim 1 to an acid catalyst to provide a reaction mixture; and
exposing the reaction mixture to reaction temperature;
wherein the cyclic dithioacetal compound has a structure according to Formula II 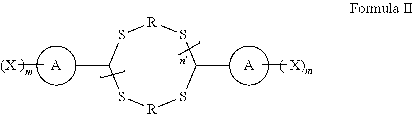
wherein
ring A is an aromatic ring system;
each R independently is selected from aliphatic, aromatic, heteroaliphatic, or any combination thereof; or
each R independently is selected from an R′ group, wherein
R′ has a structure according to a Formula A, —R^{c}—S—R^{d}—S—R^{c}—, wherein each R^{c} is selected from the groups recited for R, and R^{d} is a group having a Formula B, —R^{c}—S[CH₂]_{q}O—Ar—C(Me₂)-Ar—O[CH₂]_{q}S—R^{c}—, or a Formula C, —R^{c}—S[CH₂]_{q}Y[CH₂]_{q}S—R^{c}—, wherein for Formula B, each Ar is an aromatic ring system; for both Formulas B and C, each q independently is an integer selected from 1 to 10; and for Formula C, Y is selected from an amide group, a carbamide group, or a sulfonyl group;
each X independently is selected from an electron-withdrawing group or an electron-donating group;
n′ is an integer ranging from 1 to 8; and
m is an integer ranging from 0 to 5; provided that
(i) if ring A is phenyl and m is 0, then R is not —(CH₂)₆—, —(CH₂CH₂O)₂CH₂CH₂—, -Ph-S-Ph-;
(ii) if ring A is phenyl, m is 1, and X is para-methoxy, then R is not —(CH₂)₆—, —(CH₂CH₂O)₂CH₂CH₂—-Ph-S-Ph-, —CH₂C(O)O(CH₂)₄OC(O)CH₂—, or —(CH₂)₂C(O)O(CH₂)₄OC(O)(CH₂)₂—;
(iii) if ring A is phenyl, m is 1, and X is para-OCH₂CH═CH₂, then R is not —(CH₂CH₂O)₂CH₂CH₂—;
(iv) if ring A is phenyl, m is 1, and X is para-OH, then R is not —(CH₂CH₂O)₂CH₂CH₂—;
(v) if ring A is phenyl, m is 1, and X is para-Cl, then R is not —(CH₂CH₂O)₂CH₂CH₂—; and/or
(vi) if ring A is phenyl, m is 1, and X is para-NO₂, then R is not —(CH₂CH₂O)₂CH₂CH₂—.

25. The method of claim 24, wherein the method further comprises;
(i) combining the polymer with a solvent and wherein the reaction temperature is the refluxing temperature of the solvent, or
(ii) combining the polymer with a solvent and wherein the solvent is a solvent in which the cyclic dithioacetal compound is soluble.

26. (canceled)

27. The method of claim 24, wherein the acid catalyst and reaction temperature promote ring-closing depolymerization of the linear polymer to provide the cyclic dithioacetal compound.

28. The method of claim 24, wherein the acid catalyst is a Lewis acid catalyst selected from BF₃, BCl₃, FeCl₃, AlCl₃, GaCl₃, InCl₃, SbCl₃, SbCl₅, BiCl₃, TiCl₄, ZnCl₂, ZrCl₄, SnCl₄, HfCl₄, Zn(OTf)₂, Cu(OTf)₂, Sn(OTf)₂, or any combination thereof; or a Brønsted acid catalyst selected from p-TsOH, CF₃COOH, 10-camphorsulfonic acid, or any combination thereof.

29. A method for making a recycled polymer, comprising exposing a cyclic dithioacetal compound of claim 11 to an acid catalyst to provide the recycled polymer, wherein the acid catalyst is a Lewis acid catalyst selected from BF₃, BCl₃, FeCl₃, AlCl₃, GaCl₃, InCl₃, SbCl₃, SbCl₅, BiCl₃, TiCl₄, ZnCl₂, ZrCl₄, SnCl₄, HfCl₄, Zn(OTf)₂, Cu(OTf)₂, Sn(OTf)₂, or any combination thereof; or a Brønsted acid catalyst selected from p-TsOH, CF₃COOH, 10-camphorsulfonic acid, or any combination thereof.

30. (canceled)

31. The method of claim 29, wherein the acid catalyst promotes entropy-driven ring-opening polymerization of the cyclic dithioacetal compound to provide the recycled polymer.

32. A method, comprising:
exposing a linear polymer having a structure according to Formula I to a first acid catalyst to promote ring-closing depolymerization to provide a cyclic dithioacetal compound having a structure according to Formula II; and
exposing the cyclic dithioacetal compound to reaction conditions sufficient to promote entropy-driven ring-opening polymerization to provide a recycled linear polymer having a structure according to Formula I;
wherein Formula I is 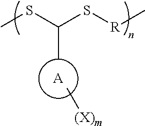
and
ring A is an aromatic ring system;
each R independently is selected from aliphatic, aromatic, heteroaliphatic, or any combination thereof; or
each R independently is selected from an R′ group, wherein
R′ has a structure according to a Formula A, —R^{c}—S—R^{d}—S—R^{c}—, wherein each R^{c} is selected from the groups recited for R, and R^{d} is a group having a Formula B, —R^{c}—S[CH₂]_{q}O—Ar—C(Me₂)-Ar—O[CH₂]_{q}S—R^{c}—, or a Formula C, —R^{c}—S[CH₂]_{q}Y[CH₂]_{q}S—R^{c}—, wherein for Formula B, each Ar is an aromatic ring system; for both Formulas B and C, each q independently is an integer selected from 1 to 10; and for Formula C, Y is selected from an amide group, a carbamide group, or a sulfonyl group;
each X independently is selected from an electron-withdrawing group or an electron-donating group;
n is an integer selected to provide an Mₙ ranging from 5,000 g/mol to 120,000 g/mol; and
m is an integer ranging from 0 to 5; and
wherein Formula II is 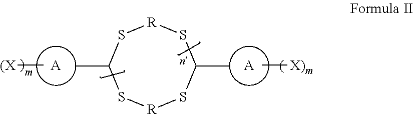
wherein
ring A is an aromatic ring system;
each R independently is selected from aliphatic, aromatic, heteroaliphatic, or any combination thereof; or
each R independently is selected from an R′ group, wherein
R′ has a structure according to a Formula A, —R^{c}—S—R^{d}—S—R^{c}—, wherein each R^{c} is selected from the groups recited for R, and R^{d} is a group having a Formula B, —R^{c}—S[CH₂]_{q}O—Ar—C(Me₂)-Ar—O[CH₂]_{q}S—R^{c}—, or a Formula C, —R^{c}—S[CH₂]_{q}Y[CH₂]_{q}S—R^{c}—, wherein for Formula B, each Ar is an aromatic ring system; for both Formulas B and C, each q independently is an integer selected from 1 to 10; and for Formula C, Y is selected from an amide group, a carbamide group, or a sulfonyl group;
each X independently is selected from an electron-withdrawing group or an electron-donating group;
n′ is an integer selected from 1 to 8; and
m is an integer ranging from 0 to 5.
